(19) United States
(12) Patent Application Publication
(10) Pub. No.: US 2017/0029812 A1
(43) Pub. Date: Feb. 2, 2017

(54) GENETICALLY ENGINEERED POLYMER LIBRARIES AND METHODS OF USING THEM

(71) Applicant: LOS ALAMOS NATIONAL SECURITY, LLC, Los Alamos, NM (US)

(72) Inventors: Jennifer S. Martinez, Dixon, NM (US); Devin W. Close, Los Alamos, NM (US); Antonietta M. Lillo, Los Alamos, NM (US); Eva Rose Murdock Balog, Saco, ME (US); Csaba Kiss, Los Alamos, NM (US); Reginaldo C. Rocha, Los Alamos, NM (US); Andrew R.M. Bradbury, Santa Fe, NM (US); Koushik Ghosh, Kingsport, TN (US); Prakash Sista, Evansville, IN (US); Hsing-Lin Wang, Los Alamos, NM (US); Charlie E. Strauss, Los Alamos, NM (US)

(21) Appl. No.: 15/115,212

(22) PCT Filed: Jan. 28, 2015
(86) PCT No.: PCT/US15/13266

§ 371 Date: Jul. 28, 2016

Related U.S. Application Data
(60) Provisional application No. 61/932,436, filed on Jan. 28, 2014.

(51) Int. Cl. C12N 15/10; C07K 7/06; C07K 1/04; C07K 7/08

(52) U.S. Cl. CPC C12N 15/1037; C07K 7/08; C07K 7/06; C07K 1/047; C07K 2319/00

(57) ABSTRACT

Disclosed herein are methods of identifying polymers having particular biological or physical characteristics from in vivo generated libraries of polymers. Methods of generating in vivo polymer libraries are also described. Polymers identified using the methods of the invention are also described.

### CROSS-REFERENCE TO RELATED APPLICATION(S)

This application is a national phase application under 35 U.S.C. §371 of International Application No. PCT/US2015/013266 filed Jan. 28, 2015, which claims the priority benefit of U.S. Provisional Application No. 61/932,436 filed Jan. 28, 2014, the entire contents of both of which are incorporated herein by reference.

### GOVERNMENT RIGHTS

This invention was made with government support under Contract No. DE-AC52-06NA25396 awarded by the U.S. Department of Energy. The government has certain rights in the invention.

### INCORPORATION BY REFERENCE

The instant application contains a Sequence Listing which has been submitted electronically in ASCII format and is hereby incorporated by reference in its entirety. Said ASCII copy, created on Apr. 10, 2015, is named S133186_002_sequence_listing.TXT and is 64,250 bytes in size.

### TECHNICAL BACKGROUND

Functional polymers and methods of identifying the same from polymer libraries are described herein.

### BACKGROUND

Advances in biomaterial design have provided the cellular precursors for advanced tissue engineering, where developing novel cellular niches that influence cell fate are highly desired. The idea is to provide the correct combination of cellular cues to promote self-renewal, to direct differentiation, or maintain pluripotency. This, in effect, is functional mimicry of the microenvironment normally afforded by the extracellular matrix (ECM), a complex network of hundreds of proteins, including collagen, fibronectin, elastin, growth factors, and ECM-modifying enzymes that coordinate to influence cell survival, shape and polarity, motility, and differentiation.

Unfortunately, a priori design of materials that mimic the ECM is extremely challenging due to lack of a full understanding of ECM biology, inability to fully emulate ECM complexity, and difficulties in building and screening large libraries of polymers. Use of biological components extracted from ECM is limited due to cost, compatibility (species-to-species cross-reactivity), and lack of precise control over composition. Application of synthetic materials generated using combinatorial polymers chemistry (isopropylacrylamide, polyacrylate, polyethylene glycol, etc.), which are commonly used as cellular niches, are limited by potential bioincompatibility, lack of biological sites, and polydispersity. In addition, the inability to create massive polymer libraries and a means to rapidly identify functional polymers (no ID-tag) limits their use beyond knowledge-based rational design. Additionally, despite the enormous combinatorial potential for adhesive peptide motifs, only a handful of sequences, and almost exclusively the RGD motif, have been used in biomaterials development, likely because most developed sequences are context dependent and not easily transferable to new polymer/materials scaffolds and because it is difficult to screen large libraries when synthetic polymers are used as scaffolds.

### SUMMARY

Disclosed herein are methods of identifying polymers having particular biological or physical characteristics. These methods include incubating a target with a plurality of first host cells, wherein each host cell expresses at least one polymer; isolating at least one host cell expressing at least one polymer that binds to the target; isolating the DNA encoding the at least one polymer that binds to the target; transforming a plurality of second host cells with said DNA to produce a plurality of second genetically engineered host cells expressing the at least one polymer encoded by said DNA; incubating the target with the second host cells expressing the at least one polymer encoded by said DNA; isolating the second host cells expressing the at least one polymer that bind to the target; and identifying the polymers that bind to the target, wherein the identification comprises isolating and sequencing the DNA from the at least one polymer expressed on the second host cells that binds to the target.

Also disclosed herein are methods of identifying a polymer comprising: providing a polymer library comprising a plurality of host cells, wherein each host cell expresses at least one polymer; applying a selective pressure to the polymer library; screening the polymer library to identify one or more polymers that respond to the selective pressure; and identifying the sequence of the polymer that confers the response to the selective pressure. A polymer can be identified by providing a plurality of host cells, wherein the host cells are genetically engineered to express at least one polymer; applying a selective pressure to the plurality of host cells expressing at least one polymer; screening the plurality of host cells expressing at least one polymer to identify one or more polymers that respond to the selective pressure; and identifying the sequence of the polymer that confers the response.

Disclosed herein are methods of identifying polymers having particular biological or physical characteristics. These methods include incubating a target with a plurality of first display systems, wherein the first display systems contain at least one polymer exposed on the surface thereof; isolating at least one first display system that contains at least one polymer that binds to the target; isolating the DNA encoding the at least one polymer that binds to the target; introducing the DNA into a plurality of second display systems to produce a plurality of second display systems expressing the at least one polymer on the surface thereof; incubating the target with the second display systems; isolating the second display systems expressing the at least one polymer that bind to the target; and identifying the polymers that bind to the target, wherein the identification comprises isolating the DNA encoding the at least one polymer from the second display systems that bind to the target and sequencing the DNA.

Also disclosed are polymers having the formula (X)(VPGIG)₂₅ wherein X is HCRGDGWLCTDK; SARYVWYNCVPIRIWR; HYYGRHWWLFHVLNYP; GYYMFSRL; GYWHYGQL; APRFRFGTMYDA; VVVERKKC; GYYMFSRL; GYWHYGQL; WHFGSLTP; APRFRFGTMYDA; WNLEPQMD; MFYEMLREWSP; RYSFGALEPISE; WKLWPMGAVPS; WYFGKME; WVLFPLGGVWS; VVVERKKC; CLLqVPWGTGTRFLTA; LCASHPLDqPVY; CHWFPRSS; FSHFVVRVNNMR; SRVDRVMV; RTWWDATTLNDY; RSAASRqKTVVV; EDPLQDGMKFqCAKVS; or LANEWqED; and wherein q represents the TAG codon that encodes Gln in E. coli. In some embodiments, the disclosed polymers further comprise an N-terminal AG, a C-terminal GSG, or both.

Polymers identified according to the described methods are also described, as are polymer libraries generated and used within the scope of the claimed methods.

### BRIEF DESCRIPTION OF THE DRAWINGS

The summary, as well as the following detailed description, is further understood when read in conjunction with the appended drawings. For the purpose of illustrating the disclosed methods, polymers, and polymer libraries, there are shown in the drawings exemplary embodiments of the methods, polymers, and polymer libraries; however, the methods, polymers, and polymer libraries is not limited to the specific embodiments disclosed. In the drawings:

FIG. 1, comprising FIGS. 1A-1D, represents (A) plasmid used in phage display and (B) plasmid used in yeast display. (C) Schematic diagram of how sEL polymers are displayed on the yeast surface.

FIG. 2, comprising FIGS. 2A-2E, illustrates an exemplary Short Elastin (sEL) display and sEL-based libraries. (A) sEL polymers are displayed on both M13 bacteriophage (through genetic fusion to the surface protein pIII) and S. cerevisiae yeast (through genetic fusion to the surface protein Aga2). The orientation of the displayed polymer is shown with X indicating the location of insertion of randomized amino acid sequence. Amino acids are randomized using the codon NNK such that every phage or yeast displays a unique X-sEL sequence. (B) Ion Torrent (IT) deep sequencing was used to compare expected and observed amino acid abundance in the naïve and displayed libraries to ensure lack of bias. (C) Progressive enrichment of integrin-binding polymers using two rounds of phage display selection followed by two rounds of yeast display sorting using FACS. Displayed sEL alone does not bind to the integrin and very little binding is observed for the naïve, or Non-selected (NS), library. Significant enrichment is observed for integrin-binding X-sEL polymers (85.5% of the population binds after two rounds each of phage selection and sorting). The integrin binding population is sorted and sequenced using IT to determine the X-sEL sequences that bind to the integrin. (D) The most abundant sequence, a5b1sEL223, binds specifically to the α₅β₁ integrin and not to α₁β₁ or αᵥβ₃ integrins when displayed on yeast. (E) When produced as soluble protein, a5b1sEL223 binds the native integrin on the surface of MSC cells in a similar manner to RGD4C-sEL, whereas sEL alone does not.

FIG. 3, comprising FIGS. 3A-3C, represents sEL polymers selected against MSC cells using phage display and sequenced using IT-seq. (A) The top ten sEL sequences ranked by order of abundance after the final round of panning against Adipose-derived MSCs (AD-MSCs). Relative abundance for each sequence within each round indicated showing enrichment over the course of selection. Sequences in bold are polymers tested as purified protein for binding against MSC cells with “sEL polymer ID” being sequences with ID that bound to MSC cells. (B) Similar to panel A, but for selection against Bone-derived MSCs (BD-MSCs). Lowercase “q” represents positions with the TAG codon that encodes a Glutamine in the E. coli strains used. (C) Exemplary purified sEL polymers tested for binding against AD-MSC cells using FACS. sEL polymers contain an SV5 tag that allows observation of binding to MSC cells when stained with an α-SV5-PE antibody.

FIG. 4, comprising FIG. 4A-4H, represents (A) MSC on sEL-coated well; (B) MSC on pep46-coated well, the aggregates range from 80 to 200 μm diameter; (C) MSC aggregates stained with anti aggrecan-dylight 488; (D) MSC aggregates stained with Safranin O with (left) or without (right) chondroitinase pre-treatment.

FIG. 5, comprising FIGS. 5A-5B, represents exemplary studies showing that MSC stem cells form spheroids when seeded on the mscsELp46 polymer. (A) Comparing MSC cells seeded and grown on borosilicate coated with sEL, no coat, or mscsELp46, then analyzed after 21 days of growth. In the case of the no coat, chondrogenesis media (CM) was added to stimulate chondrogenesis and serve as a chondrogenesis positive control. Cells were stained with Formazan to improve contrast. MSCs seeded on mscsELp46 exhibit spheroid formation consistent with chondrogenesis while cells seeded on sEL exhibit typical MSC growth. (B) Seeding on mscsELp46 accelerates spheroid formation when combined with CM. When CM was added, MSCs seeded on mscsELp46 formed spheroids at a much higher rate than the non-coated chondrogenesis control, with spheroids being observed as early as 2 days after addition of CM whereas spheroids are not observed in the no coat+CM for nearly two weeks.

FIG. 6, comprising FIGS. 6A-6D, represents exemplary studies showing the production of specific proteins by spheroids grown on mscsELp46 is suggestive of chondrogenesis. (A) Safranin O stains Glycosaminoglycan (GAG) molecules produced on the surface of cells, a hallmark of ECM production and differentiation. The robust staining was supported by a GAG quantification assay where the total GAG produced was normalized to cells using the MTT assay. Spheroids grown on mscsELp46 stain with antibodies against (B) Sox9, (C) Collagen IIA, and (D) Aggrecan at levels comparable to the chondrogenesis control. Normal goat sera (Control sera), is used to monitor non-specific interaction of the primary and/or secondary reagents with the spheroids.

FIG. 7 represents an exemplary purification of sEL and a few selected polymers that bind integrins and MSCs.

FIG. 8 represents and exemplary study showing cross reactivity of downselected polymers for different integrins.

FIG. 9, comprising FIGS. 9A-9B, represents an exemplary study showing that GRGDSPsEL does not interact with (A) AD-MSC cells or with (B) recombinant human integrins. The GRGDSP sequence is expected to bind the a5b1 integrin.

FIG. 10, comprising FIGS. 10A-10B, represents an exemplary study testing the selected binding motif from mscp46sEL for binding to AD-MSC cells out of the context of sEL. (A) Minimal binding was observed to MSC cells at increasing concentration of a biotinylated version of the peptide. (B) In a slightly different assay, cells were incubated with non-biotinylated peptide at 2× and 10× molar concentration relative to mscp46sEL and binding to mscp46sEL did not change indicating the free peptide cannot outcompete mscp46sEL for binding.

FIG. 11, comprising FIGS. 11A-11E, illustrates (A) an exemplary outline of the procedure for generating the specific modified amino acid. The FGE enzyme was added to an sEL polymer with the N-terminal sequence LCPTSR. FGE modified the C residue to fGly that reacted with hydrazide to generate a covalent bond to the hydrazide containing molecule, in this case biotin-hydrazide. This led to an sEL polymer that was selectively biotinylated. (B) purification of recombinant FGE. (C) Purification of recombinant LCPTSR-sEL. (D) Western blot using stredtavidin (binds to biotin) for detection. Different reactions set up with either the LCPTSR-sEL polymer, a control sEL polymer (vegfm), with and without FGE, and with and without biotin-hydrazide. An obvious band at ˜16.5 KDa demonstrated specific, FGE-dependent biotinylation of LCPTSR-sEL. (D) An anti-SV5 western blot showed that comparable levels of protein were used in the reactions. FGE and sEL polymers contain an SV5 epitope.

FIG. 12, comprising FIGS. 12A-12B, represents an exemplary multi-stimuli responsive genetically encoded polymer (ELP that has embedded functional moieties). Here the functional moiety was an amine modified p-polyphenylene oligomer positioned within the polymer backbone, doppped into a hydrogel or utilized as a crosslinker to cast the hydrogel. (A) The sEL polymer hydrogel was doped with amine modified p-polyphenylene. The resultant polymer shows altered (e.g. emergent) properties not found with the OPPV alone. The resultant hydrogel demonstrated optical and physical changes as a function of pH and temperature. (B) A COOH-sEL polymer hydrogel where the crosslinker was the amine-OPPV (K-sEL). The resultant hydrogel had altered optical response as a function of pH, temperature and mechanical strain (i.e. the fluorescence turned on with addition of strain).

FIG. 13, comprising FIGS. 13A-13B, represents an exemplary genetically encoded polymer (ELP) that has embedded functional moieties. Here the functional moiety was a transition metal (A) or lanthanides (B). Note that the properties of the resultant polymers can be tuned by varying the density and nature of the metal complex.

FIG. 14, comprising FIGS. 14A-14B, represents an exemplary genetically encoded polymer (ELP) with embedded semi-conducting moiety (A). The genetically encoded polymer can be utilized as either the dielectric or semi-conducting region of a flexible electronics.

FIG. 15, comprising FIGS. 15A-15C, represents an exemplary characterization of a genetically encoded polymer (k-SEL). (A) Exemplary demonstration of the physical changes of a hydrogel made from amine-ELP (K-SEL) as a function of temperature and dynamic light scattering of the polymer (non-hydrogel form) as a function of pH. (B) Exemplary SEM micrographs of K-sEL hydrogels showing macroporous structure and intrinsic microporosity. (C) Exemplary tensile strength measurements showing storage and loss moduli and dynamic shear viscosity as a function of frequency for K-sEL hdyrogesl measure at 4° C. and 37° C.

### DETAILED DESCRIPTION

The disclosed methods, polymers, and polymer libraries may be understood more readily by reference to the following detailed description taken in connection with the accompanying figures, which form a part of this disclosure. It is to be understood that the disclosed methods, polymers, and polymer libraries are not limited to the specific methods, polymers, and polymer libraries described and/or shown herein, and that the terminology used herein is for the purpose of describing particular embodiments by way of example only and is not intended to be limiting of the claimed methods, polymers, and polymer libraries.

Similarly, unless specifically otherwise stated, any description as to a possible mechanism or mode of action or reason for improvement is meant to be illustrative only, and the disclosed methods, polymers, and polymer libraries are not to be constrained by the correctness or incorrectness of any such suggested mechanism or mode of action or reason for improvement.

Where the disclosure describes or claims a feature or embodiment associated with a polymer, polymer library or a method of identifying, generating or using the same, it is appreciated that such a description or claim is intended to extend these features or embodiments to embodiments in each of these contexts. For example, and without intending to be limiting, features or embodiments relating to the described polymers or polymer libraries apply equally to methods of identifying, generating or using said polymers or polymer libraries.

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

When values are expressed as approximations, by use of the antecedent “about,” it will be understood that the particular value forms another embodiment.

It is to be appreciated that certain features of the disclosed methods, polymers, and polymer libraries which are, for clarity, described herein in the context of separate embodiments, may also be provided in combination in a single embodiment. Conversely, various features of the disclosed methods, polymers, and polymer libraries that are, for brevity, described in the context of a single embodiment, may also be provided separately or in any subcombination.

As used herein, the singular forms “a,” “an,” and “the” include the plural.

The term “plurality,” as used herein, means more than one.

Methods of Identifying Polymers with Pre-Selected or Useful Biological or Physical Property

Disclosed herein are methods of identifying a polymer having a pre-selected or useful biological or physical property. Suitable pre-selected or useful biological or physical properties include, but are not limited to, the ability of the polymer to bind to a target, or the ability of the polymer to respond to the application and/or change in temperature, force, pressure, pH, catalysis, optical excitation, current, voltage, or any combination thereof.

Methods of identifying a polymer comprise: subjecting a plurality of first display systems to a selective pressure, wherein the first display systems contain at least one polymer exposed on the surface thereof; isolating at least one first display system that contains at least one polymer that responds to a selective pressure; isolating the DNA encoding the at least one polymer that responds to the selective pressure; and identifying the polymer that responds to the selective pressure, wherein the identification step comprises isolating the DNA encoding the at least one polymer that responds to the selective pressure and sequencing the DNA.

In some aspects, the method can further comprise: introducing the DNA into a plurality of second display systems to produce a plurality of second display systems expressing the at least one polymer on the surface thereof; subjecting the second display systems to a selective pressure; isolating at least one second display system that contains at least one polymer that responds to a selective pressure; and isolating the DNA encoding the at least one polymer that responds to the selective pressure; wherein the introducing, subjecting, and isolating steps occur prior to the identifying step.

In some embodiments, the methods comprise: incubating a target with a plurality of first display systems, wherein the first display systems contain at least one polymer exposed on the surface thereof; isolating at least one first display system that contains at least one polymer that binds to the target; isolating the DNA encoding the at least one polymer that binds to the target; and identifying the polymers that bind to the target, wherein the identifying step comprises isolating the DNA encoding the at least one polymer from the first display systems that bind to the target and sequencing the DNA.

In some embodiments, the method can be performed as an iterative process. For example, and without intending to be limiting, the methods can comprise: incubating a target with a plurality of first display systems, wherein the first display systems contain at least one polymer exposed on the surface thereof; isolating at least one first display system that contains at least one polymer that binds to the target; isolating the DNA encoding the at least one polymer that binds to the target; introducing the DNA into a plurality of second display systems to produce a plurality of second display systems expressing the at least one polymer on the surface thereof; incubating the target with the second display systems; isolating the second display systems expressing the at least one polymer that bind to the target; and identifying the polymers that bind to the target, wherein the identification comprises isolating the DNA encoding the at least one polymer from the second display systems that bind to the target and sequencing the DNA.

In some embodiments, the methods comprise: incubating a target with a plurality of first host cells, wherein each host cell expresses at least one polymer; isolating at least one host cell expressing at least one polymer that binds to the target; and identifying the at least one polymer that binds to the target, wherein the identification comprises isolating and sequencing the DNA from the at least one polymer expressed on the second host cells that bind to the target.

In other embodiments, the method can be iterative, comprising, for example: incubating a target with a plurality of first host cells, wherein each host cell expresses at least one polymer; isolating at least one host cell expressing at least one polymer that binds to the target; isolating the DNA encoding the at least one polymer that binds to the target; transforming a plurality of second host cells with said DNA to produce a plurality of second host cells expressing the at least one polymer encoded by said DNA; incubating the target with the second host cells expressing the at least one polymer encoded by said DNA; isolating the second host cells expressing the at least one polymer that binds to the target; and identifying the at least one polymer that binds to the target, wherein the identification comprises isolating and sequencing the DNA from the at least one polymer expressed on the second host cells that bind to the target.

Suitable display systems include, but are not limited to, host cells, bacteriophage, virions, and ribosomes (using, for example, in vitro display). As used herein, the term “host cell” refers to any cell in which the polymer can be introduced and expressed upon the surface thereof. Preferred host cells include, but are not limited to, viruses, yeasts, or bacteria. In some aspects, the host cells can be a virus. In other aspects, the host cells can be a yeast. In yet other aspects, the host cells can be a bacteria.

The first host cells and second host cells can be the same or different. In some embodiments, the first host cells and second host cells can be the same. For example, in some aspects, that first host cells and second host cells can be phage. In some aspects, that first host cells and second host cells can be yeast. In some aspects, that first host cells and second host cells can be bacteria. In other embodiments, the first host cells and second host cells can be different. For example, in some aspects, the first host cells can be phage and the second host cells can be yeast. In other aspects, the first host cells can be phage and the second host cells can be bacteria. In other aspects, the first host cells can be yeast and the second host cells can be phage. In other aspects, the first host cells can be yeast and the second host cells can be bacteria. In other aspects, the first host cells can be bacteria and the second host cells can be phage. In other aspects, the first host cells can be bacteria and the second host cells can be yeast.

Within the scope of the disclosed methods, at least one display system will be manipulated to express, or “display,” a polymer. For example, host cells can be manipulated to express a polymer on the surface thereof. Preferably, the host cell is manipulated using genetic engineering techniques known to those in the art. The use of phage and yeast display provides, for example, directed generation of polymers. This directed generation enables the identification of polymers having a desired functionality, including, for example, the ability of a target to bind to the polymer. This directed generation also enables the identification of design rules in order to predictably generate polymers having a pre-selected functionality.

Phage and yeast display techniques are generally understood in the art. In exemplary embodiments of the disclosed methods, a polymer is “displayed” on the surface of a host cell (i.e. phage or yeast) by fusing the gene that codes for that polymer to the gene of a surface protein of phage or yeast. As a result, a host cell can be genetically engineered to display a particular polymer. According to the methods, a plurality of host cells can be generated, wherein each host cell of the plurality expresses a distinct, that is, different, polymer. This collection of host cells thus includes a plurality of distinct polymers. This in vivo combinatorial generation results in each host cell carrying one polymer and the DNA that encodes for that polymer's sequence (enabling self-replication). This results in an efficient identification-tag system (one-polymer/one-phage/one-DNA).

It will be understood by those in the art that a host cell can display a single, distinct polymer, having a particular chemical structure. Within the scope of the disclosed methods, a single host cell can additionally display multiple copies of that single polymer.

A collection of host cells expressing distinct polymers thus provides a “polymer library.” These polymer libraries can be used with the methods of the invention to identify and select polymers having particular functionality, for example, a particular biological function or physical property.

As used herein, the term “selective pressure” refers to functional assays that are designed to reduce the library size from millions of polymers to a few polymers having one or more specific functions. The “selective pressure” used in the assays of the invention are pressures that have been correlated with a specific function, such as temperature or light responsiveness. Selective pressures include, but are not limited to, binding to a target (including but not limited to affinity and adhesion), temperature, force, pressure, pH, catalysis, optical excitation, current, voltage, or any combination thereof. In some aspects, for example, the selective pressure can comprise binding to a target. In some aspects, the selective pressure can comprise application of and/or a change in temperature. In some aspects, the selective pressure can comprise application of and/or a change in force. In some aspects, the selective pressure can comprise application of and/or a change in pressure. In some aspects, the selective pressure can comprise application of and/or a change in pH. In some aspects, the selective pressure can comprise catalysis. In some aspects, the selective pressure can comprise application of and/or a change in light (for example, optical excitation). In some aspects, the selective pressure can comprise application of and/or a change in current. In some aspects, the selective pressure can comprise application of and/or a change in voltage. In some aspects, the selective pressure can comprise application of and/or a change in any combination of the above selective pressures.

Accordingly, “subjecting a plurality of first display systems to a selective pressure” includes, but is not limited to, incubating a target with a plurality of first display systems, applying or changing a temperature, applying or changing a force, applying or changing a pressure, applying or changing a catalyst, applying or changing light, applying or changing a current, applying or changing a voltage, or any combination thereof.

“Respond to the selective pressure” refers to a new, and/or a change in, function, activity, shape, confirmation, aggregation, solubility, assembly, etc. For example, in some embodiments the response can be binding to cells. In other embodiments the response can be inducing the differentiation of cells. In other embodiments, the response can be insoluble phase transition. In still other embodiments, the response can be intrinsic fluorescence. In some embodiments the response can be magnetism. In some embodiments the response can be metal binding. In some embodiments the response can be the release of contents from polymer. In some embodiments the response can be the catalysis of specific reaction. In yet other embodiments, the response can be any combination of the above responses. Each of these, and numerous other, responses can be tested alone or in combination.

As used herein, the term “target” refers to any biological or non-biological molecule, cell, protein, peptide, nucleic acid molecule, carbohydrate, plastic, chemical, drug, pharmaceutical, therapeutic, and the like, that can bind to any one of the polymers expressed on an individual display system, such as a host cell. In some embodiments, the target can be a cell. For example, in some aspects, phage cells expressing the polymers can be incubated with a cell of interest to evaluate whether the polymer, expressed on the surface of the phage, can bind to the cell. Phage cells, for example, can be incubated with Adipose-derived (AD) mesenchymal stem cells (MSCs), bone-derived (BD) MSCs, or any suitable cell. In other embodiments, the target can be a protein. For example, in some aspects, a protein can be incubated with the first host cells expressing the at least one polymer. In yet other embodiments, the target can be a peptide. Binding can be achieved using any of the methods known in the art.

As used herein, the term “polymer” refers to molecules comprising, or alternatively consisting of, one or more repeating blocks, wherein the blocks comprise, or alternatively consists of, a protein-like moiety (made up of amino acids, amino acid variants, or both), a functional moiety, or any combination thereof, and wherein at least one block is repeated two or more times. In embodiments wherein the blocks are amino acids and/or amino acid variants, each block will comprise, or alternatively consist of, at least two amino acids, amino acid variants, or a combination thereof. For example, a block can comprise, or alternatively consist of, at least two amino acids. Alternatively, a block can comprise, or alternatively consist of, at least two amino acid variants. Further still, a block can comprise, or alternatively consist of, at least two amino acid and amino acid variant combinations. The disclosed polymers are genetically engineered (created at the DNA level) and are expressed/exposed on the surface of a host cell or other suitable display system. Polymers generated in this manner are also referred to herein as in vivo polymers. Suitable polymers are discussed herein.

One skilled in the art understands that there are numerous ways to genetically engineer a host cell to express a particular polymer. For example, in some embodiments, DNA encoding a polymer can be introduced into a host cell by any suitable method known in the art, including, but not limited to, transformation. In some aspects, DNA encoding a polymer can be transiently expressed by a host cell, such that the DNA is not stably integrated into the genome of the host cell. In some aspects, DNA encoding a polymer is stably integrated into the genome of the host cell. For example, DNA encoding a polymer can be genetically fused to the DNA encoding a host cell protein that is expressed on the surface of the host cell. In this aspect, the polymer remains in the host cell genome and is expressed on the cell surface through successive generations of the host cell. In some embodiments, DNA encoding a polymer can be fused to or genetically engineered to the AGA2 protein of a yeast cell. The DNA encoding the polymer can be stably transformed into the cell without being integrated into the genome of the host, as, for example, nongenomic DNA.

Host cells can display the genetically-expressed polymer as a plurality of individual polymers. Alternatively, the individual polymers expressed on the host cell can self-assemble into higher ordered structures.

The steps of the described methods can be performed one or more times. For example, the step of incubating a target with a plurality of first host cells, wherein each host cell expresses at least one polymer can be performed at least one time. Preferably, during incubation, the target will bind to at least one of the polymers expressed on a host cell. In some embodiments, more than one distinct, expressed polymer will bind to the target. After incubation, the host cell (or cells) expressing the polymer (or polymers) that bind(s) to the target is isolated. After the host cell is isolated, the DNA encoding the at least one polymer that binds to the target is isolated. A plurality of second host cells can be transformed with the isolated DNA.

In some embodiments, the incubating a target with a plurality of first host cells, each host cell expressing at least one polymer, isolating at least one host cell expressing the at least one polymer that binds to the target, and the isolating the DNA encoding the at least one polymer that binds to the target is repeated one or more times. Such techniques are known in the art and are sometimes referred to as “biopanning.” Such techniques can be used to reduce the number of polymers to those that are functional, i.e. that bind to the target of interest.

The disclosed methods can further comprise testing the polymers as monoclones. In this aspect, the polymer can be exposed on the surface of an additional display system and screened for a property of interest. For example, a host cell can be transformed with the identified polymer and screened for a property of interest. Thus, after the polymers that bind to the target are identified, the DNA encoding the identified polymers can be re-introduced into a display system, and evaluated for a property of interest. In this aspect, testing the polymers as monoclones can be used to verify the functional polymers.

As used herein, “property of interest” can be any functional or physical activity, including, but not limited to, binding to a cell of interest, inducing the differentiation of a cell of interest, catalysis, conductance, phase change, conformational change, insoluble phase transition, intrinsic fluorescence, stimuli-responsive, elasticity differences, or any combination thereof. In some aspects, the property of interest can be binding to a cell of interest. In some aspects, the property of interest can be inducing differentiation of a cell of interest. In some aspects, the property of interest can be binding to a cell of interest and inducing differentiation of a cell of interest. In some aspects, the property of interest can be catalysis. In some aspects, the property of interest can be conductance. In some aspects, the property of interest can be phase change. In some aspects, the property of interest can be a conformational change. In some aspects, the property of interest can be insoluble phase transition. In some aspects, the property of interest can be intrinsic fluorescence. In some aspects, the property of interest can be stimuli-responsive. In some aspects, the property of interest can be elasticity difference. In yet other aspects, the property of interest can be any combination of the above functional or physical activities.

Cells of interest include any cell tested for the ability to bind to the polymer and/or differentiate as a result of binding to the polymer. In some embodiments, the cell of interest can be stem-cells. In other embodiments, the cells of interest can be osteoblasts. In yet other embodiments, the cell can be chondrocytes.

In some embodiments, cellular extracts can be used in place of a host cell. For example, a polymer or polymer library can be introduced into a host cell, the host cell can be lysed, and the cellular extract can be tested for a property of interest.

As used herein, the term “screen” refers to the differentiation of functional from non-functional polymers. Screening includes, but is not limited to, binding (e.g. adhesion to a protein or cell), insoluble phase transition (e.g. by precipitation and centrifugation), intrinsic fluorescence (e.g. sorting by flow cytometry), change in conformation, catalysis, conductance, phase change, or any combination thereof. In some aspects, screening can comprise evaluating binding. In some aspects, screening can comprise evaluating a change in solubility. In some aspects, screening can comprise evaluating a change in light emission. In yet other aspects, screening can comprise any combination of the above functions.

Regardless of the selective pressure or screening method used, selection and screening of polymers generated using the disclosed methods can be an iterative process (performed for rounds) where the library pool is sequentially enriched with functional polymers, i.e. biopanning. In some embodiments each repeated round can comprise the same selection and screening. For example, a plurality of host cells collectively expressing a polymer library can be tested for the ability to bind AD-MSCs, a process that can be repeated multiple times. In alternative embodiments, each repeated round can comprising a different selection and screening. For example, a plurality of host cells collectively expressing a polymer library can be tested for the ability to bind a cell of interest in the first round, and in a subsequent round tested for the ability to respond to changes in temperature.

Techniques known in the art can be used to identify the polymer sequence that is responsible for conferring the response. For example, the identifying the polymer sequence can comprise isolating the DNA from the host cells that express at least one polymer that responds to the selective pressure and sequencing the DNA to identify the at least one polymer that responds to the selective pressure.

Methods of identifying a polymer from a polymer library are also disclosed. The polymer library can consist of a plurality of display systems, each expressing a distinct polymer on the surface thereof. For example, in some embodiments, the method of identifying a polymer from a polymer library can comprise: providing a polymer library comprising a plurality first display systems, wherein each first display system expresses at least one polymer; applying a selective pressure to the polymer library; screening the polymer library to identify one or more polymers that respond to the selective pressure; and identifying the sequence of the polymer that confers the response.

In other embodiments, the polymer library can comprise a plurality of host cells, each host cell expressing at least one distinct polymer. For example, the method of identifying a polymer from a polymer library can comprise: providing a polymer library comprising a plurality of host cells, each host cell expressing a distinct polymer; subjecting the polymer library to a selective pressure; and selecting the polymer within the polymer library that responds to the selective pressure.

Any of the above disclosed embodiments for identifying a polymer from one or more display systems are equally applicable to identifying a polymer from a polymer library.

Also provided here are kits for identifying polymers, said kits comprising nucleic acids for the formation of polymer libraries, one or more display systems, and instructions for performing the disclosed methods.

The methods disclosed herein provide, for example, the ability to identify and select polymers that are biologically, thermally, or optically responsive. The methods and the disclosed polymers can be used in a host of applications, including, but not limited to, cell niches for regenerative medicine, drug-delivery, diagnostics, tissue engineering, 3D tissue scaffolds, molecular electronics for use in, for example, photovoltaics, sensing devices, and LEDs. Furthermore, the methods can be used to identify and select polymers that are cell adhesive (cell-specific biorecognition ligands), biodegradable, stimuli-responsive (e.g. release signals or nutrients as a function of cellular-growth state or in response to specific light wavelengths), have spatial and temporal presentation of biorecognition signals and the ability to organize on the nanometer to micron scale, or any combination thereof.

### Genetically Engineered Polymers

The disclosed polymers can comprise, or alternatively consist of, a number of suitable protein like moieties, including, but not limited to, elastin-like protein (ELP) moieties, silk-like protein (SLP) moieties, silk-elastin-like proteins (SELP) moieties, resilin-like protein moieties, helical bundle moieties, fl-sheet forming moieties, semi-random moieties or any combination thereof. Preferably, the polymers produced according to the methods of the invention include ELP, SLP, and/or SELP moieties.

Elastin-like protein (ELP) moieties comprise, or alternatively consist of, one or more blocks of Valine-Proline-Glycine-X-Glycine (VPGXG), where X can be any amino acid other than Proline. In some embodiments, the polymers comprise at least one VPGXG unit, wherein X is any amino acid except proline. Thus, in some embodiments, the polymer can have 1 VPGXG unit in addition to the at least one block that is repeated two or more times. The VPGXG can be the at least one block that is repeated two or more times. For example, in some embodiments, the polymer can have 2 VPGXG units. In some embodiments, the polymer can have 5 VPGXG units. In some embodiments, the polymer can have 10 VPGXG units. In some embodiments, the polymer can have 15 VPGXG units. In some embodiments, the polymer can have 20 VPGXG units. In some embodiments, the polymer can have 25 VPGXG units. Polymers can comprise, or alternatively consist of, these repeating VPGXG units alone or in combination with other protein-like or functional moieties as disclosed herein.

ELPs are biocompatible. Depending on the number of repeating units and other moieties within the polymer, the properties of an individual ELP can be modulated. The repeated block sequence can give rise to elastomeric properties such as an inverse temperature transition and high resiliency. Polymers containing ELP moieties can be modified to contain blocks of exogenous sequence, while still retaining their elastomeric properties. The number of VPGXG blocks can be varied resulting in different temperatures of transition, and the blocks can be mixed with other sequence blocks to generate co-block polymers with novel binding and functional properties. Further, polymers containing ELP moieties can be made into hydrogels and other three dimensional matrices through addition of specific conjugation sites, as briefly described in Trabbic-Carlson, K.; Setton, L. A.; Chilkoti, A. “Swelling and mechanical behaviors of chemically cross-linked hydrogels of elastin-like polypeptides.” Biomacromolecules 2003, 4, (3), 572-80.

Short elastin (sEL) moieties refer to ELP scaffolds comprising 25 repeats of the sequence Valine-Proline-Glycine-Isoleucine-Glycine [(VPGIG)₂₅]. sEL moieties provide an ideal length for inverse transition at physiologically relevant temperatures, enabling relatively straightforward purification of protein from bacteria and interesting transitive properties between 4° C. and 37° C. Polymers containing sEL moieties can be expressed and purified from E. coli in high quantities (>50 mg/L culture) to >98% homogeneity.

Silk-like protein moieties refers to one or more blocks of GAGAGS, GA, GGX, GPGGX, poly-A.

Silk-elastin-like protein (SELP) moieties refers to [(S)ₓ(E)_{y}]ₙ blocks, wherein S is SLP and E is ELP.

Resilin-like protein moieties refers to one or more GGRPSDSYGAPGGGN blocks or GYSGGRPGGQDLG blocks.

The disclosed polymers can contain random amino acids on the N-terminus or C-terminus of the ELP, sEL, SLP, or SELP moieties. In some embodiments, the polymers can contain 8 random amino acids on the N-terminus or C-terminus of the ELP, sEL, SLP, or SELP moieties. In other embodiments, the polymers can contain 12 random amino acids on the N-terminus or C-terminus of the ELP, sEL, SLP, or SELP moieties. In other embodiments, the polymers can contain 16 random amino acids on the N-terminus or C-terminus of the ELP, sEL, SLP, or SELP moieties. Having random amino acids at the N-terminus or C-terminus of the moieties allows for the creation of peptides with random amino acids throughout the peptides (i.e. between the repeating units) as well as at the N-terminus and C-terminus of the peptides. These random amino acids can be added to the polymer by numerous methods known in the art. For example, each amino acid can be encoded by the DNA sequence NNK, wherein N is any base and K is a G or T.

The disclosed polymers can also comprise, or alternatively consist of, one or more functional moieties. Functional moieties include, but are not limited to, one or more blocks that impart the ability to bind to a target (including but not limited to affinity and adhesion) and or respond to the application and/or change in temperature, force, pressure, pH, catalysis, optical excitation, current, voltage, or any combination thereof. Accordingly, suitable functional moieties include, but are not limited to biologically-reactive groups, optically-reactive groups, thermo-reactive groups, photo-reactive groups, catalytic groups, stimuli-responsive groups, conductive groups, semi-conductive groups, or any combination thereof.

Suitable functional moieties can be random, semi-random, or known peptides of natural and non-natural amino acids. Functional moieties can be amino acid based, non-amino acid based, or a combination thereof. In some aspects, the functional moiety can be amino acid based. For example, functional moieties can be genetically engineered as a random peptide, lysine, aspartate, to name a few. In other aspects, the functional moiety can be non-amino acid based. Non-amino acid based functional moieties include, for example, metal complexes, oligomers of conducting, semiconducting, or optical polymers, and catalysts, to name a few. In yet other embodiments, the functional moiety can be both amino acid based and non-amino acid based.

The reactivity of the disclosed polymers will depend on a number of factors including, but not limited to, differences in packing, location, and density of functional moieties and protein moieties. For example, it has been demonstrated that phase transitions, leading to changes in scaffold packing, are dependent on the amino acid sequence (block), and the number of repeating homo and hetero blocks (Urry, D. W. “Physical chemistry of biological free energy transduction as demonstrated by elastic protein-based polymers” J. Phys. Chem. B. 101, (1997), 11007).

Functional groups can be added by a variety of techniques known in the art, including, but not limited to, genetic engineering and chemical modification.

In some embodiments, the functional moiety can comprise conjugated oligomers such as oligomeric phenylene vinylene and thiophene. The optical properties of a conjugated polymer can be regarded as the collective optical property of the ensemble of conjugated oligomers. As previously shown, these properties are dominated by the intermolecular interactions between oligomers and should be better controlled in in vivo polymers. (Wang, C.-C., et al., “Thermochromism of a Poly(phenylene vinylene): Untangling the Roles of Polymer Aggregate and Chain Conformation” J. Phys. Chem. B 113 (2009), 16110; Tang, Z., et al., “Study of the non-covalent interactions in Langmuir-Blodgett films: An interplay between pi-pi and dipole-dipole interactions” Thin Solid Films 516 (2007) 58). A suite of polyphenylene vinylene and thiophene oligomers with well-defined structures and functionality can be synthesized using Sonogashira and Heck coupling reactions, as previously published (Tang, Z., et al., “Synthesis and characterization of amphiphilic phenylene ethynylene oligomers and their Langmuir-Blodgett films” Langmuir 22, (2006), 8813; Hassan, J., et al., “Palladium-catalyzed coupling reactions towards the synthesis of well-defined thiophene-oligomers” Organometalic Chem. 687, (2003), 280; Babudri, F., et al. “Synthesis of conjugated oligomers and polymers: the organometallic way” J. Mat. Chem. 14, (2004), 11). The suite of oligomers allows for the selection of the optimal oligomer structure and molecular weight, while preserving the polymer packing.

In some embodiments, the functional moiety can comprise transition-metal complexes. These components combine highly efficient photoactivity and unique redox flexibility that can control useful chemistry (e.g. light-triggered delivery of NO for cell death). By altering substituent groups (R′) and ancillary ligands (L), transition, actinides, or lanthanide metal complexes can be formed with strong metal-to-ligand charge-transfer absorptions that can be modulated across the Vis to near-IR regions and their M^{II}/M^{III} redox potentials can be adjusted by a few or hundreds of mV.

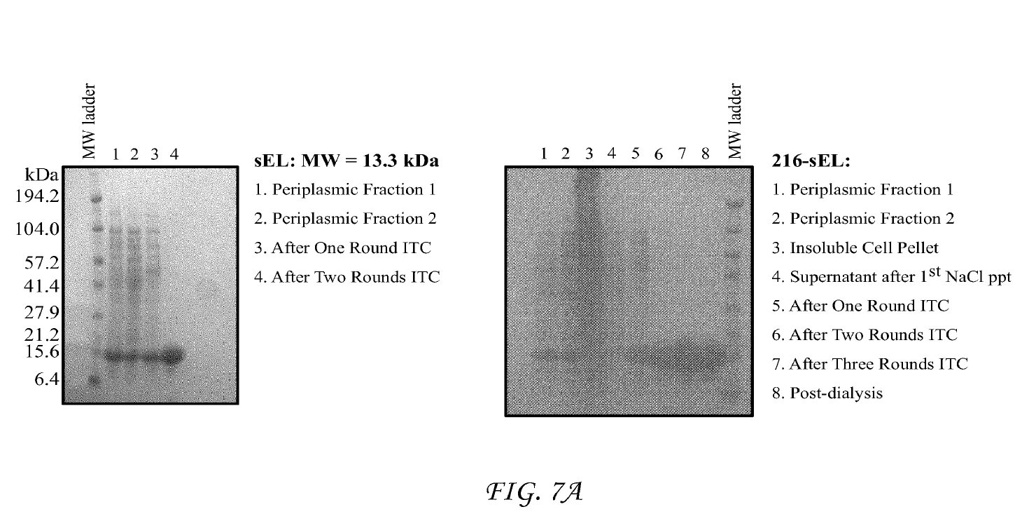

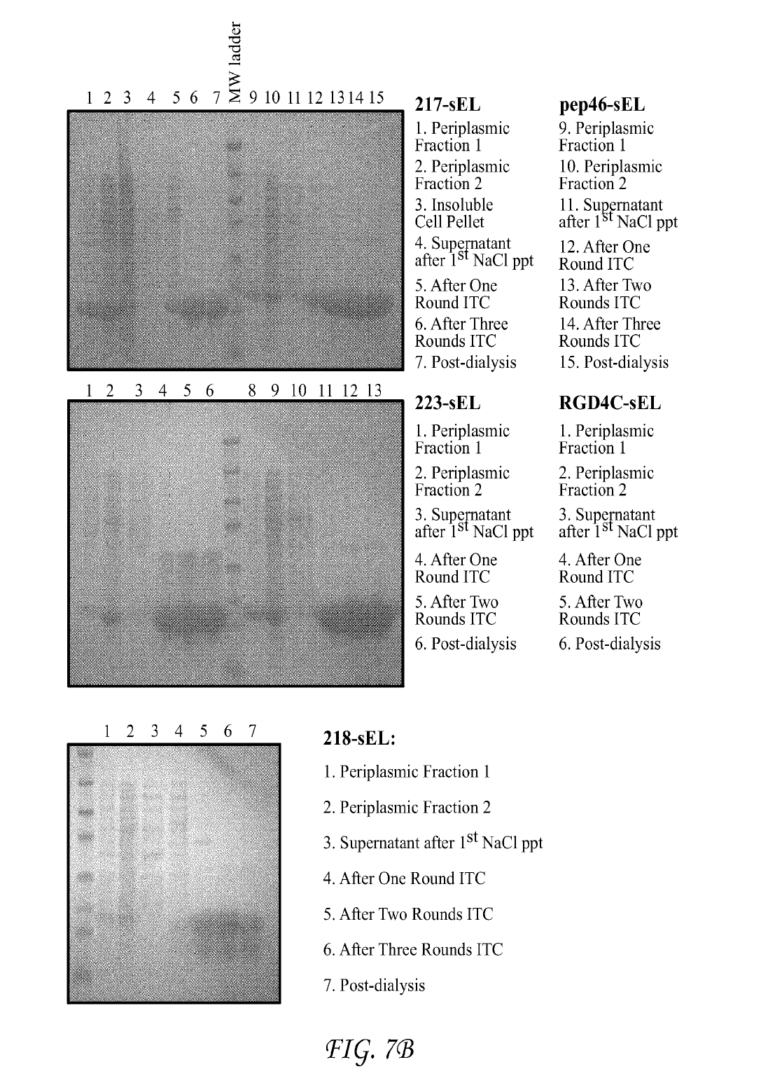

In yet other embodiments, the functional moiety can be added to the nascent polymer either biologically (e.g. utilizing a specialized tRNA, modified amino acid within a cell deprived of that natural amino acid) or by post-translational modifications. For example, in vivo polymers can be post-translationally modified with carboxylate, amine, or alkyl halide reactivity. Furthermore, the above types of functionally active components will have end carboxylic acid or amine groups to undergo amide linkage to complementary side-chain groups (—NH₂/—COOH) of amino acids (e.g. Lys, Asp, or Glu), through EDC/NHS chemistry. Fortunately, in vivo polymer scaffolds are largely devoid of these amino acids unless specifically introduced via genetic engineering. An additional suite can be produced with alkyl halides that can react with cysteines. Having both primary functionalities allows future libraries to be modified with two distinct functional moieties. Either the biological or post-translational modifications can be done not only to a purified polymer (as above), but also to a mixture of polymers. Cysteine residues can be modified using enzymatic means as well. This is accomplished using prior art described by Bertozzi, et al. (Rabuka, D., Rush, J. S., DeHart, G. W., Wu, P., Bertozzi, C. R. “Site-specific chemical protein conjugation using genetically encoded aldehyde tags” Nature Protocals 7(6) 2012) with the procedure summarized in FIG. 7. Briefly, a specific sequence (LCPTSR) can be introduced into the polymer that is recognized by a Formylglycine Generating Enzyme (FGE). FGE then selectively modifies that Cys residue to formylglycine (fGly) that can then react with hydrazide groups enabling site specific labeling with hydrazide containing molecules. An example is shown in FIG. 7 where a specific polymer (AG-X-GSG(VPGIG)₂₅ X=LCPTSR), is modified at its N-terminus with a biotin-hydrazide molecule. The site can be incorporated (at the DNA level) at any location in the sequence of genetically encoded polymers.

Polymers can also comprise, or alternatively consist of, a combination of protein-like moieties and functional moieties. For example, and without intent to be limiting, suitable polymers can contain: one protein moiety and one functional moiety; two or more protein moieties and one functional moiety, wherein the two or more protein moieties can be the same or different protein moieties; one protein moiety and two or more functional moieties, wherein the two or more functional moieties can be the same or different functional moieties; or two or more protein moieties and two or more functional moieties, wherein the two or more protein moieties can be the same or different and wherein the two or more functional moieties can be the same or different.

The polymers can be created from constrained amino acids (for example, Y, G, S). In yet other embodiments, polymers can be created using computer modeling. Computer modeling enables the prediction of folding of polymers in silico (producing materials by design). The design of stable and functional helical bundles, including the switchable control of functionality and structure, has been demonstrated in Korendovych, I. V., et al. “Design of a switchable eliminase.” Proc. Nat. Acad. Sci. USA 108, (2011), 6823. This design has been encoded into the MSL modeling software package described in Zhang, Y., et al. “Experimental and computational evaluation of forces directing the association of transmembrane helices.” J. Amer. Chem. Soc. 131, (2009), 11341; Berger, B. W., et al. “Consensus motif for integrin transmembrane helix association.” Proc. Nat. Acad. Sci. USA 107, (2010), 703. MSL provides parameterizations of helix bundles that account for 95% of all known natural helix bundle structures and maintain canonical interactions within 1 Å, using a highly restricted subset of helix bundle parameter space. Structural design of loop regions, bundle-bundle docking and functionalization, as proposed for ELP, SELPs and helical bundles, requires sophisticated and free-form backbone modeling. For this Rosetta Software can be used for constrained modeling, loop modeling, and core algorithms.

Also provided are polymer libraries, produced according to the described methods. In some embodiments, the polymer libraries can comprise a plurality of display systems, each display system containing on the surface thereof at least one distinct polymer. In other embodiments, the polymer libraries can comprise a plurality polymers isolated from a display system. It is preferred that each polymer within the polymer library comprises, or alternatively consists of, different protein-like moieties and/or functional moieties, or a different orientation thereof.

The disclosed polymer libraries can contain polymers having any of the above disclosed characteristics. Accordingly, the above disclosed polymer characteristics, including but not limited to the disclosed protein-like moieties and functional moieties, are equally applicable to the disclosed polymer libraries.

Large, diverse libraries of in vivo polymers can be created with incorporated functional moieties, such as optical- and bio-reactive moieties, and functional polymers can be identified using a genetic technique akin to evolution (phage and yeast display).

The polymer libraries can comprise homopolymers or heteropolymers. Thus, in some embodiments, the library can comprise homopolymers comprising, or alternatively consisting of, ELP moieties, for example [(VPGIG)ₓ(F)_{y}]ₙ where x, y and n vary within the polymer library and F is a functional moiety. In other embodiments, the library can comprise homopolymers comprising, or alternatively consisting of, SLP moieties, for example [(GAGAGS)ₓ(F)_{y}]ₙ, where x, y and n vary within the polymer library and F is a functional moiety. In yet other embodiments, the libraries can comprise heteropolymers comprising, or alternatively consisting of, ELP moieties, for example [(VPGIG)ₓ(VPGVG)_{y}F_{z}]ₙ, where x, y and n vary within the polymer library and F is a functional moiety. In other embodiments, the libraries can comprise heteropolymers comprising, or alternatively consisting of, SELP-coblock polymers, for example [(GAGAS)ₓ(VPGIG)_{y}F_{z}]ₙ, where x, y and n vary within the polymer library and F is a functional moiety.

Using purely biological (genetically encoded) polymers (i.e. in vivo polymers), such as those described herein, enables not only generation of massive libraries (>10⁷ variants), but provides a means to screen massive libraries using methods such as phage and yeast display.

By creating libraries of in vivo polymers with side groups of functional moieties, polymers can be created that can adapt, sense, or react to their environment, converting one type of signal into another signal or functionality.

Also disclosed herein are polymers having the formula (X)(VPGIG)₂₅. In some embodiments X is HCRGDGWLCTDK. In other embodiments, X is SARYVWYNCVPIRIWR. In other embodiments, X is HYYGRHWWLFHVLNYP. In other embodiments, X is GYYMFSRL. In other embodiments, X is GYWHYGQL. In other embodiments, X is APRFRFGTMYDA. In other embodiments, X is VVVERKKC. In other embodiments, X is GYYMFSRL. In other embodiments, X is GYWHYGQL. In other embodiments, X is WHFGSLTP. In other embodiments, X is APRFRFGTMYDA. In other embodiments, X is WNLEPQMD. In other embodiments, X is MFYEMLREWSP. In other embodiments, X is RYSFGALEPISE. In other embodiments, X is WKLWPMGAVPS. In other embodiments, X is WYFGKME. In other embodiments, X is WVLFPLGGVWS. In other embodiments, X is VVVERKKC. In other embodiments, X is CLLqVPWGTGTRFLTA. In other embodiments, X is LCASHPLDqPVY. In other embodiments, X is CHWFPRSS. In other embodiments, X is FSHFVVRVNNMR. In other embodiments, X is SRVDRVMV. In other embodiments, X is RTWWDATTLNDY. In other embodiments, X is RSAASRqKTVVV. In other embodiments, X is EDPLQDGMKFqCAKVS. In other embodiments, X is LANEWqED. As used herein, “q” represents the TAG codon that encodes Glu in E. coli.

In some embodiments, the disclosed polymers having the formula (X)(VPGIG)₂₅ can further comprise an N-terminal AG sequence, a C-terminal GSG sequence, or both. For example, in some embodiments X can be AGHCRGDGWLCTDKGSG. In other embodiments, X can be AGSARYVWYNCVPIRIWRGSG. In other embodiments, X can be AGHYYGRHWWLFHVLNYPGSG. In other embodiments, X can be AGGYYMFSRLGSG. In other embodiments, X can be AGGYWHYGQLGSG. In other embodiments, X can be AGAPRFRFGTMYDAGSG. In other embodiments, X can be AGVVVERKKCGSG. In other embodiments, X can be AGGYYMFSRLGSG. In other embodiments, X can be AGGYWHYGQLGSG. In other embodiments, X can be AGWHFGSLTPGSG. In other embodiments, X can be AGAPRFRFGTMYDAGSG. In other embodiments, X can be AGWNLEPQMDGSG. In other embodiments, X can be AGMFYEMLREWSPGSG. In other embodiments, X can be AGRYSFGALEPISEGSG. In other embodiments, X can be AGWKLWPMGAVPSGSG. In other embodiments, X can be AGWYFGKMEGSG. In other embodiments, X can be AGWVLFPLGGVWSGSG. In other embodiments, X can be AGVVVERKKCGSG. In other embodiments, X can be AGCLLqVPWGTGTRFLTAGSG. In other embodiments, X can be AGLCASHPLDqPVYGSG. In other embodiments, X can be AGCHWFPRSSGSG. In other embodiments, X can be AGFSHFVVRVNNMRGSG. In other embodiments, X can be AGSRVDRVMVGSG. In other embodiments, X can be AGRTWWDATTLNDYGSG. In other embodiments, X can be AGRSAASRqKTVVVGSG. In other embodiments, X can be AGEDPLQDGMKFqCAKVSGSG. In other embodiments, X can be AGLANEWqEDGSG. As used herein, “q” represents the TAG codon that encodes Glu in E. coli.

### Methods of Generating Polymer Libraries

Also disclosed are methods of generating polymer libraries. Said methods can comprise manipulating a display system to express, or “display,” a polymer library. For example, host cells can be manipulated to express a polymer on the surface thereof. Preferably, the host cell is manipulated using genetic engineering techniques known to those in the art. The use of phage and yeast display provides, for example, directed generation of polymers. This directed generation also enables the identification of design rules in order to predictably generate polymers having a pre-selected functionality. In some embodiments, the methods of generating the polymer library comprise generating a plurality of host cells wherein each host cell expresses a distinct polymer.

The disclosed methods (generation of polymer libraries, selection of polymers, etc) and polymers generated therefrom have advantages over previously described methods. For example, the disclosed methods can be used to generate polymers from a plurality of pre-selected DNA sequences, each known to code for a particular chemical structure. As a result, the disclosed methods can generate and/or identify polymers having a controlled structure and/or function, extraordinary monodispersity, that is, homogeneous molecular weight. These polymers can also have well-defined stereochemistries, sequences, reproducible folding and packing, and the ability to form hierarchical assemblies. Additionally, polymers generated and/or identified by the disclosed methods can be regioregular, that is, have the same repeating units, resulting in defined packing of the backbone (scaffold). Such polymers can also have defined functional moieties (i.e. bio- or optically-reactive moieties). Further, the genetic engineering of polymers can enable the creation of libraries of millions of related, yet distinct, polymers.

### Examples

### Cells, Phage, and Growth Media

E. coli BL21 Gold cells were used for protein expression and purification. E. coli SS320 cells were used for initial transformation of libraries, and E. coli DH5αF′ or Omnimax (Life Technologies) cells were used for phage production and display. Unless otherwise noted, all E. coli strains were grown in 2xyT (Gibco) liquid media or solid agar+glu (3% glucose) and appropriate selective antibiotic (both Carbenicillin (amp) and Kanamycin (kan) used at 50 ug/ml and Tetracycline (tet) added to 15 ug/ml).

Bacteriophage were produced in DH5αF′ cells following a 20:1 MOI infection with M13KO7 (New England Biolabs) or KM13 helper phage and grown at 30° C. at 250 rpm for 16-24 hours in 2xyT media containing amp and 25 ug/ml kan (0.5× concentration). Bacteria cells were spun down (˜10,000×g, 10 min) and phage were precipitated twice from the culture supernatant in a 20% volume of 2.5 M NaCl+20% PEG-8000. Phage were quantified by infecting 5 mls of DH5αF′ cells grown to OD 0.5 with 10 ul of phage solution then incubating without shaking at 37° C. for 30 minutes. The solution is spun down and cells resuspended in 1 ml of 2xyT media. Serial dilutions (1:10) were made and titrations spotted on 2xyT+amp/glu in duplicates. The spots with the highest number of resolvable colonies were counted and the phage concentration extrapolated based on dilution.

S. cereviseae EBY100 cells were used for yeast display and grown in YPD at 30° C. when selection was required and SD/CAA+kan+tet when selection to maintain pDNL7 was required. Yeast display was also performed using the plasmid pDNL6. When displayed on pDNL7, the polymer is fused N-terminal to the Aga2 gene, and in pDNL6, the polymer is fused C-terminal to the Aga2 gene. For display, EBY100 yeast were grown for 18-30 hours in SD/CAA media followed by 1:100 dilution into SGR/CAA+kan+tet induction media and grown at 20° C., 250 rpm for 20-72 hours.

Adipose derived Human Mesenchymal Stem Cells (AD-MSC) were obtained from ATCC® (PCS-500-011) and Bone-derived MSC cells (BD-MSC) were obtained from PromoCell, and all reagents were obtained from Life Technologies unless otherwise noted. MSC cells were grown (high humidity, 5% CO₂, 37° C.) incubator and passaged according to the manufacturer's recommendations. In short, MSC cells were grown in Mesenchymal Stem Cell media (ATCC® PCS-500-030)+2% v/v FetalClone III Serum (FCS; HyClone)+1× Anti-Anti+1× Glutamax. Adherent cells were detached for passaging or experiments between 60-90% confluency using TrypLE reagent for 5-12 minutes followed by addition of MSC media. Live cells were differentiated from dead cells using Trypan Blue staining, counted using a hemocytometer, and seeded based on live cell counts. For passaging, cells were typically plated at 8,000-30,000 live cells/cm² and not used beyond passage five. Up to passage five, no difference was observed in standard MSC cell surface markers (CD44, CD90, CD73, and CD105) as determined using the human MSC analysis kit (BD Biosciences).

### Immunoctytochemistry.

The expression of Coll2A, Sox9 and Aggrecan in AD-MSC grown on various selected and control polymers, or differentiated into chondrocytes (as described above), was assayed by immunocytochemistry using the following protocol. Cell were washed twice with 600 μL PBS, fixed and permeabilized with a mixture of methanol and acetone (7:3) at −20° C. for 5 minutes, washed again 3 times with 600 μL PBS, and blocked with 10% donkey serum (Abcam, ab7475) and 0.1% tween20 in PBS (blocking buffer). Primary goat antibody, either goat anti aggrecan (R&D Systems, AF1220), anti collagen2A (Santa Cruz Biotechnology, sc-7764), anti sox9 (R&D Systems, AF3075), or normal goat (Santa Cruz Biotechnology, sc-2028) was added at final concentration of 10 μg/mL in 5-fold diluted blocking buffer, followed by overnight incubation at 4° C. and 3 washes with 0.03% BSA, 0.03% skimmed milk, 0.03% fish gelatin and 0.01% tween20 in PBS (wash buffer). Alexa Fluor® 488 donkey anti-goat secondary antibody (Life Technologies, A-11055) was added to a final concentration of 8 μg/mL, in 5-fold diluted blocking buffer, followed by incubation at RT for 1 hr, and 3 washes with wash buffer. DAPI was added at a final concentration of 2.9 μM in PBS, followed by incubation at RT for 10 min and 3 washes in PBS. When spheroids were above 200 μm in diameter, they tended to float even after fixation. In those instances it was necessary to perform the washing steps by centrifugation of the supernatant (1000 rpm for 3 min). Cells were observed by fluorescence microscopy.

### Safranin O Staining

The presence of acidic proteoglycan (ex: chondroitin and dermatan sulfate) indicative of chondrogenesis was assayed by safranin O staining. Cells were washed twice with Hunk's balanced salt solution (HBSS), fixed with 4% parafolmaldehyde in PBS overnight at 4° C., washed 3× with HBSS, blocked in 0.1% BSA in PBS for 2 hours at RT and stained with a water solution of safranin O (0.1%) for 40 min at RT. Cells were washed twice with 95% ethanol and twice with 100% ethanol. Stained cells were immediately observed by microscopy. In order to determine the specificity of safranin O staining, some culture were pretreated with chondroitinase which degrades chondroitin sulfate and therefore should cause a reduction of safranin O staining intensity. Cells were washed twice with HBBS, followed by addition of chondrotinase (vendor, catalog) at a final concentration of 1 U/mL in 50 mM Tris pH8, 60 mM sodium acetate and 0.02% BSA and overnight incubation at 37° C.

### Aggrecan, GAG, MTT Assay

Quantification of production of aggrecan, as related to its release in the growth medium, and quantification of production of sulphated glycosamino glycans, was assessed by using the PG-EASIA (BioSource Europe S.A., KAP1461) and the Proteoglycan Detection (RHEUMERA, 8000) kits, respectively. Results were normalized for cell viability, by performing the two assays simultaneously with the Vybrant® MTT Cell Proliferation Assay (Molecular Probes, V-13154). The three assays were performed according to the manufacturer recommendations, with the following modifications. For the PG-EASIA assay, two different standard curves were constructed diluting the standard with the highest concentration of aggrecan (standard 5), in either MSC medium or chondrogenesis medium, to obtain two sets of standards ranging from 7.9 to 1.6 ng/mL. The two standard curves were used to extrapolate concentration of aggrecan in cell cultures grown on p46 or sEL (standards diluted in MSC medium) and in cell cultures induced to chondrogenesis (standards diluted in chondrogenesis medium). For the Proteoglycan Detection assay, the manufacturer's protocol was scaled down 5-fold. Also instead of the 1 mL 50 mM Tris-HCl pH8 prescribed for quenching the papain digestion mixture, 100 μL of a 10-fold concentrated tris solution was used, in order to increase the concentration of GAG to be detected in each sample analyzed. Standard solutions of GAG, to be used for the calibration curve, were reconstituted in a quenched papain digestion mixture of composition similar to the one used for the samples analyzed. The MTT assay was performed exactly like the manufactured recommends. A calibration curve, correlating the concentration of formazan to the number of viable cells, was constructed by using a known number of MSC cells (3000-16000) as determined by trypan blue staining and counting with an hemocytometer

### Plasmids and Library Generation

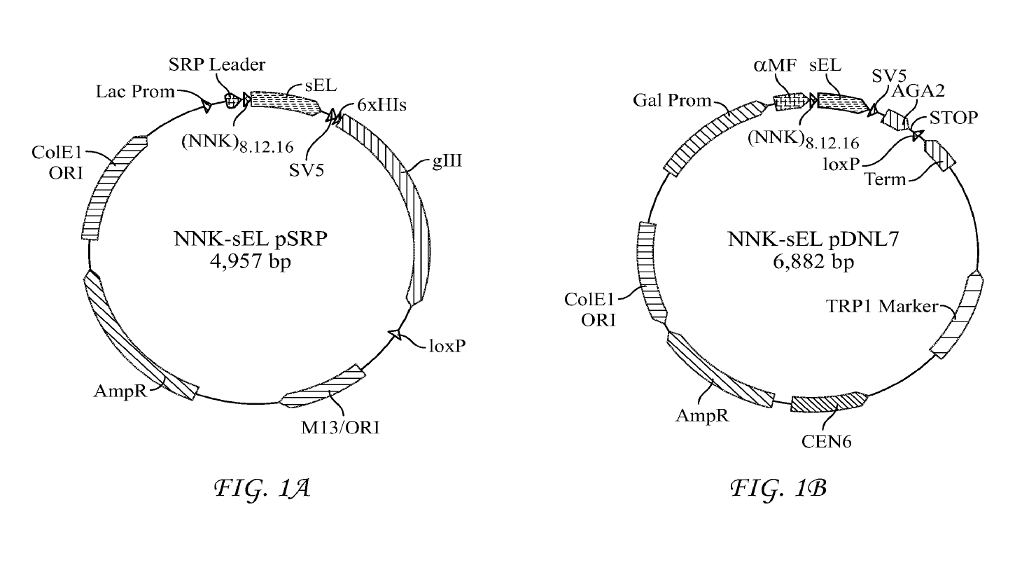

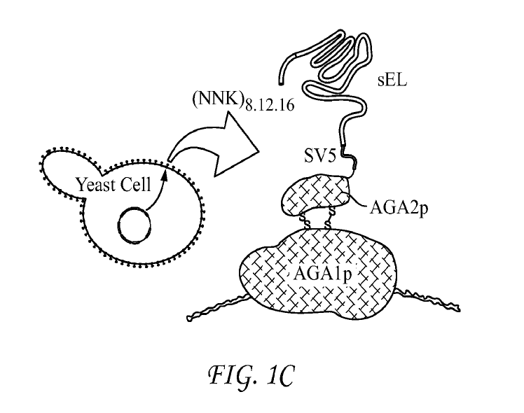

For phage display, sEL proteins are cloned into a pSRP plasmid (FIG. 1A) resulting in display of sEL polymers on the pIII protein of M13 filamentous phage. POE was used for protein production in E. coli, and pDNL7 was used for N-terminal yeast display. The plasmid pDNL6 was also tested and displayed at levels comparable to pDNL7, but pDNL7 was chosen because the orientation of the display (polymer is N-terminal to the Aga2 gene) was preferred to allow the diversity to be on the terminal end of the displayed protein fusion. The pSRP plasmid is a modified version of the pDAN5 phage display plasmid modified to contain an SRP leader sequence, described in Velappan, N. et al. A comprehensive analysis of filamentous phage display vectors for cytoplasmic proteins: an analysis with different fluorescent proteins. Nucl. Acids Res. 38, e22-e22 (2010). For yeast display, a vector was created based on a previously described plasmid pDNL6 (Ferrara, F., Listwan, P., Waldo, G. S. & Bradbury, A. R. M. Fluorescent Labeling of Antibody Fragments Using Split GFP. PLoS ONE 6 (2011)) called pDNL7 (FIG. 1B). The pDNL7 plasmid differs from pDNL6 in that the gene of interest, in this case sEL, is fused to the amino terminus of the Aga2 protein resulting in display of the polymer with the binding element or diversity, on the terminal end of the chimera (FIG. 1D).

An elastin-like polymer composed of 25 VPGIG repeats [(VPGIG)₂₅] was designed using an in-house program to randomize DNA codons while accounting for E. coli codon preferences. The gene was synthesized by GeneArt (Life Technologies), called “sEL” for “short Elastin”, and cloned into pSRP using engineering BssHII and NheI restriction endonucleases (New England Biolabs).

sEL libraries were generated using a variation on circular polymerase extension cloning (CPEC), described in (Quan, J. & Tian, J. Circular polymerase extension cloning for high-throughput cloning of complex and combinatorial DNA libraries. Nature Protocols 6, 242-251 (2011)), and non-library constructs generated using either CPEC or standard Restriction Enzyme (RE) digests using BssHII and NheI followed by ligation. Prior to cloning, PCR amplification was used to generate insert and vector fragments using Phusion High Fidelity Polymerase and associated buffers (New England Biolabs). 5′ oligos used for amplification of the libraries were synthesized and PAGE purified (IDT). All other oligonucleotides used in this study were synthesized by MWG Operon or Invitrogen (Life Technologies). Oligos used in CPEC reactions were designed to leave complimentary 5′ and 3′ ends on the Insert and Vector whose respective melting temperatures were within 1-2° C. (˜72° C.). sEL-pSRP gene architecture and oligonucleotide sequences are listed in Tables 1 and 2.

TABLE 1
Gene architecture for plasmids.

| Plasmid | Gene Feature | Sequence |
|---|---|---|
| pSRP | SRP leader | KLAKFYFKETVIMKKIWLALAG |
| | | LVLAF |
| | Linker and | SAHA*AG**GSG |
| | Insertion site | |
| | sEL | [VPGIG]₂₅VPAS |
| | SV5 tag | GKPIPNPLLGLDST |
| | His tag | HHHHHH |
| POE | pelB leader | MKYLLPTAAAGLLLLAA |
| | Linker and | SGAHA*AG**GSG |
| | Insertion site | |
| | sEL | [VPGIG]₂₅VPAS |
| | SV5 tag | GKPIPNPLLGLDST |
| | His tag | HHHHHH |
| pDNL7 | App8 leader | MRFPSIFTAVLFAASSALAAPA |
| | | NTTTEDETAQIPAEAVIDYSDL |
| | | EGDFDAAALPLSNSTNNGLSST |
| | | NTTIASIAAKEEGVQLDKR |
| | Linker and | *GAHAAG**GSG |
| | Insertion site | |
| | sEL | [VPGIG]₂₅VPAS |
| | SV5 tag | GKPIPNPLLGLDST |
| | Linker | [GGGGS]₃ |
| | Aga2 | QELTTICEQIPSPTLESTPYSL |
| | | STTTILANGKAMQGVFEYYKSV |
| | | TFVSNCGSHPSTTSKGSPINTQ |
| | | YVF |
| pDNL6 | Aga2 | QELTTICEQIPSPTLESTPYSL |
| | | STTTILANGKAMQGVFEYYKSV |
| | | TFVSNCGSHPSTTSKGSPINTQ |
| | | YVF |
| | Linker | KDNSSTIEG |
| | HA tag | RPYDVPDYALQA |
| | Linker | SGGGGSGGGGSGGGGSAR |
| | Linker and | *GAHAAG**GSG |
| | Insertion site | |
| | sEL | [VPGIG]₂₅VPAS |
| | SV5 tag | GKPIPNPLLGLDST |
| | His tag | HHHHHH |

*peptidase cleavage site
**library or control peptide insertion site. For sEL without N-terminal insertions, the GSG linker is not included.

TABLE 2
Oligonucleotide sequences.

| Name | Sequence | Description |
|---|---|---|
| NNK8sel_F | CGTTTAGCGCGCAT | 5′ oligo used for |
| | GCCGCCGGANNKNN | generating library |
| | KNNKNNKNNKNNKN | insert fragment |
| | NKNNKGGCTCTGGT | containing 8 random |
| | GTACCAGGTATCGG | codons |
| | TGTCCCCG | |
| NNK12sel_F | CGTTTAGCGCGCAT | 5′ oligo used for |
| | GCCGCCGGANNKNN | generating library |
| | KNNKNNKNNKNNKN | insert fragment |
| | NKNNKNNKNNKNNK | containing 12 random |
| | NNKGGCTCTGGTGT | codons |
| | ACCAGGTATCGGTG | |
| | TCCCCG | |
| NNK16sel_F | CGTTTAGCGCGCAT | 5′ oligo used for |
| | GCCGCCGGANNKNN | generating library |
| | KNNKNNKNNKNNKN | insert fragment |
| | NKNNKNNKNNKNNK | containing 16 random |
| | NNKNNKNNKNNKNN | codons |
| | KGGCTCTGGTGTAC | |
| | CAGGTATCGGTGTC | |
| | CCCG | |
| selsv5_R | CAGTGGGTTTGGGA | 3′ oligo used for |
| | TTGGTTTGCCGC | generating sEL |
| | | insert fragments |
| psrp_inv_F | GCGGCAAACCAATC | 5′ oligo used for |
| | CCAAACCCACTG | inverse PCR of pSRP |
| | | vector |
| psrp_inv_R | TCCGGCGGCATGCG | 3′ oligo used for |
| | CGCTAAACG | inverse PCR of pSRP |
| | | vector |
| Ion_psrp_F | CCTCTCTATGGGCA | 5′ oligo used in PCR |
| | GTCGGTGATTCTGG | amplification from |
| | CTGGCGCTGGCAG | pSRP for IT-seq |
| Ion_pdnl7_F | CCTCTCTATGGGCA | 5′ oligo used in PCR |
| | GTCGGTGATTGCCA | amplification from |
| | AAGAAGAAGGAGTC | pDNL7 for IT-seq |
| | CAG | |
| Ion_MID_R | TTCCATCTCATCCC | 3′ oligo(s) used in |
| | TGCGTGTCTCCGAC | PCR amplification |
| | TCAGNNNNNNNNNN | from pSRP |
| | | for IT-seq. |
| | GTACCCCGATCCCA | The (N)₁₀ represents |
| | GGAACT | unique barcode |
| | | sequence |
| psrptopdnl7_F | GCCAAAGAAGAAGG | Used for |
| | AGTCCAGTTAGATA | amplification |
| | AAAGAGGCGCGCTG | of sEL genes out of |
| | GCGTTTAGCGCGCA | pSRP that enables |
| | TGCCGC | recombination into |
| | | pDNL7 |
| psrptopdnl7_R | CTCCTGTTGAATCT | Used for |
| | AATCCTAATAATGG | amplification |
| | GTTTGGGATAGGCT | of sEL genes out of |
| | TTCCGCTAGCAGGT | pSRP that enables |
| | ACCCCAATCCCCGG | recombination into |
| | CACA | pDNL7 |

Vector (pSRP) was amplified in a 50 ul inverse PCR reaction: 10 ul 5×HF buffer (1×), 5 ul 2.5 mM dNTPs (250 uM each), 2.5 ul of each 10 uM oligo (0.5 uM each; Table 1), 0.5 ul 2 ng/ul sEL-pSRP template (˜1 ng), 0.5 ul 2 U/ul Phusion HF Polymerase, and 29 ul of water. Thermocycling conditions: 98° C. for 30 sec, 30 cycles of 98° C. 10 sec, 63° C. 20 sec, 72° C. 90 sec, and a final extension of 72° C. for 5 min. Insert fragment PCR was optimized to account for high GC content in a 50 ul reaction as follows: 10 ul 5×GC buffer (1×), 2.5 ul DMSO, 5 ul 2.5 mM dNTPs (250 uM each), 2.5 ul of each 10 uM oligo (0.5 uM each), 0.5 ul 2 ng/ul sEL-pSRP template (˜1 ng), 0.5 ul 2 U/ul Phusion HF Polymerase, and 26 ul of water. Thermocycling conditions: 98° C. for 30 sec, 30 cycles of 98° C. 10 sec, 72° C. 20 sec, and a final extension of 72° C. for 3 min. Following PCR, amplicons were purified by electrophoresis using a 1% Agarose gel and a gel extraction kit (Qiagen) followed by quantification using absorbance at 280 nm. For cloning of fragments into POE or pDNL7 plasmids, a PCR purification kit (Qiagen) was performed after PCR and followed by RE digest at 37° C. for insert and plasmid using manufacturer's guidelines for BssHII and NheI (New England Biolabs). Digests were followed by gel electrophoresis and extraction (Qiagen) of cut digested fragments. Ligations were set up with a 3:1 insert:vector ratio in reactions consisting of 200-600U of T4 ligase in appropriate T4 buffer (New England Biolabs) for at least 2 hours at 16° C. Ligations were inactivated at 65° C. for 20 min followed by transformation by electroporation of 1-5 ul of the ligation reactions into electrocompotent E. coli BL21 cells.

To generate libraries, gel purified insert and vector were added in a reaction mix at equimolar ratios with the following components: 10 ul 5×HF buffer (1×), 5 ul 2.5 mM dNTPs (250 uM each), 0.5 ul 2 U/ul Phusion HF Polymerase, and water to 50 ul total. 12×50 ul reactions for each library (NNK8, NNK12, or NNK16-sEL) were set up such that each reaction consisted of 335 ng (120 fmoles) of vector and equimolar concentration of insert. Thermocycling conditions were: 98° C. 30 sec, (98° C. 10 sec, ramp from 70° C. to 56° C. at 1° C./10 sec, 55° C. 40 sec, 72° C. 100 sec)×25 cycles, then 72° C. for 5 min. After cycling, each reaction was purified and concentrated using a Minielute PCR purification kit (Qiagen) followed by electroporation of 4 ul of the purified reaction in 120 ul of SS320 E. coli cells. Transformed cells were added immediately to 950 ul of pre-warmed SOC media, recovered by shaking at 37° C. for 1-1.5 hours, and followed by plating on 2xyT amp/glu agar plates. The efficiency of transformation was improved by an order of magnitude or more when the SS320 cells were freshly grown and made competent on the day of transformation without freezing. Library size was determined by plating 1:10 serial dilutions of the recovered cells and extrapolating to determine total diversity. On plates where a 1:100,000 dilution was plated, 900 cfus, 1050 cfus, and 600 cfus were counted resulting in diversities of 9×10⁷, 1.1×10⁸, and 6×10⁷ for the NNK8-, NNK12-, and NNK16-sEL libraries (respectively).

### Integrin Selection, Sorting, Binding—Combining Phage and Yeast Display to Identify Integrin Binding Polymers

To demonstrate the feasibility of selections using an ELP scaffold and that sEL libraries could be functionally displayed on phage and yeast, selection and sorting against human a5b1 integrin was performed as described in detail below. Briefly, two rounds of phage display followed by subcloning the enriched output into the yeast display vector pDNL-7 were used. The enriched library was displayed on yeast and sorted for yeast expressing NNK-sEL polymers that bind to the integrin. These polymers were sorted, regrown, and the sort was repeated. Integrins were utilized as a first protein target since their role in ECM-directed cell fate is well established. Selection and sorting was first performed against human a5b1 integrin as described below.

Recombinant human integrins used in this study were ordered from R&D Systems. Prior to use in phage or yeast display, integrins were reconstituted in phosphate buffered saline (PBS) then buffer exchanged into IPBST (0.1% v/v Tween-20, 1 mM MgCl₂, 1 mM CaCl₂ in PBS) using Micro Bio-Spin 6 Columns (Bio-Rad) for a final concentration of 0.1-0.8 uM. Integrins were conjugated with a 50-100 fold excess of biotin using EZ-Link™ Sulfo-NHS-LC-LC-Biotin (Thermo Scientific) at 22° C. for 15-60 minutes. A second buffer exchange into IPBST was performed to remove free biotin. Integrins were quantified using a NanoDrop spectrophotometer (Thermo Scientific).

Phage panning was performed using a King Fisher (Thermo Scientific) magnetic bead selection. Ten microliters of streptavadin conjugated magnetic M-280 Dynabeads (Life Technologies) per selection were blocked in Blocking Buffer (BB; 1% w/v BSA+1% w/v fish gelatin in PBS) prior to use. Phage and integrins were blocked separately in 1:1 mix of IPBST+BB on ice for 30-60 minutes. Approximately 10¹¹ blocked phage were mixed with integrins at ˜250-400 nM (for first round) or ˜100-200 nM (in second round) in a volume of ˜30 ul then brought up to 190 ul in the first well of the King Fisher plate. Phage bound to biotinylated integrins were captured on the magnetic strep beads and washed 3× in PBST and 2× in PBS followed by elution in 150 ul 0.1N HCl for 5 minutes with subsequent neutralization with 50 ul 1.5M Tris pH 8.8. Eluted phage were infected into 5 ml DH5αF′ or Omnimax at 0.5 OD600 at 37° C. for 30 min followed by plating on amp/glu agar and growth at 30° C. Bacteria were then plated on amp/glu agar plates and grown at 30° C. overnight. The following day, bacteria are scraped and frozen in 2xyT+20% glycerol at −80° C. or grown to produce phage for subsequent rounds.

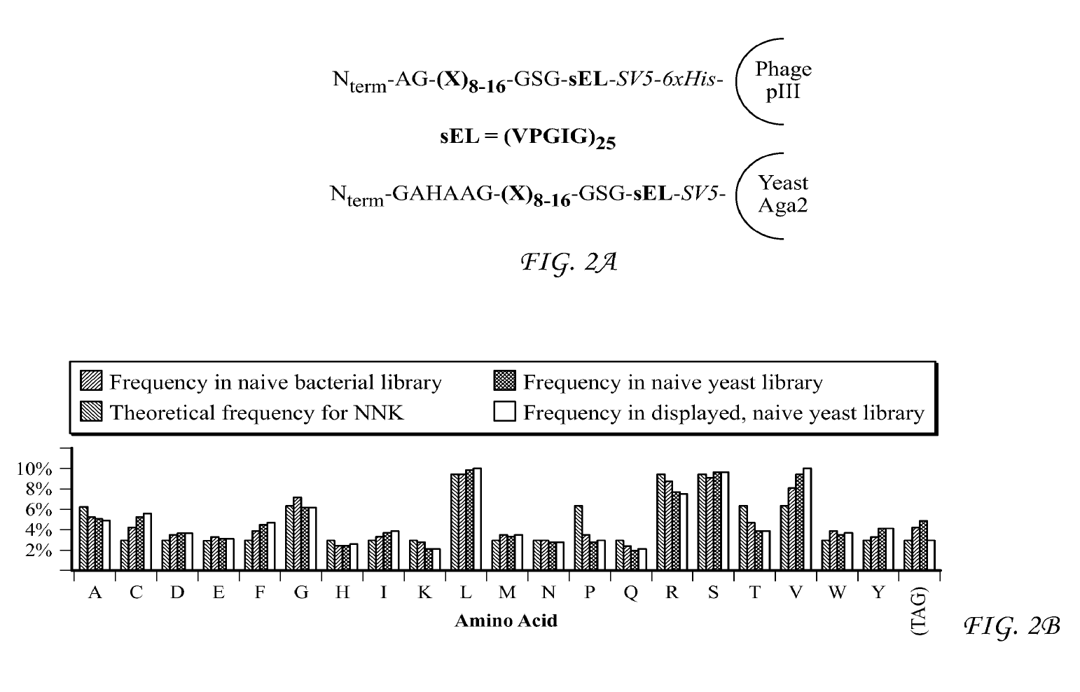

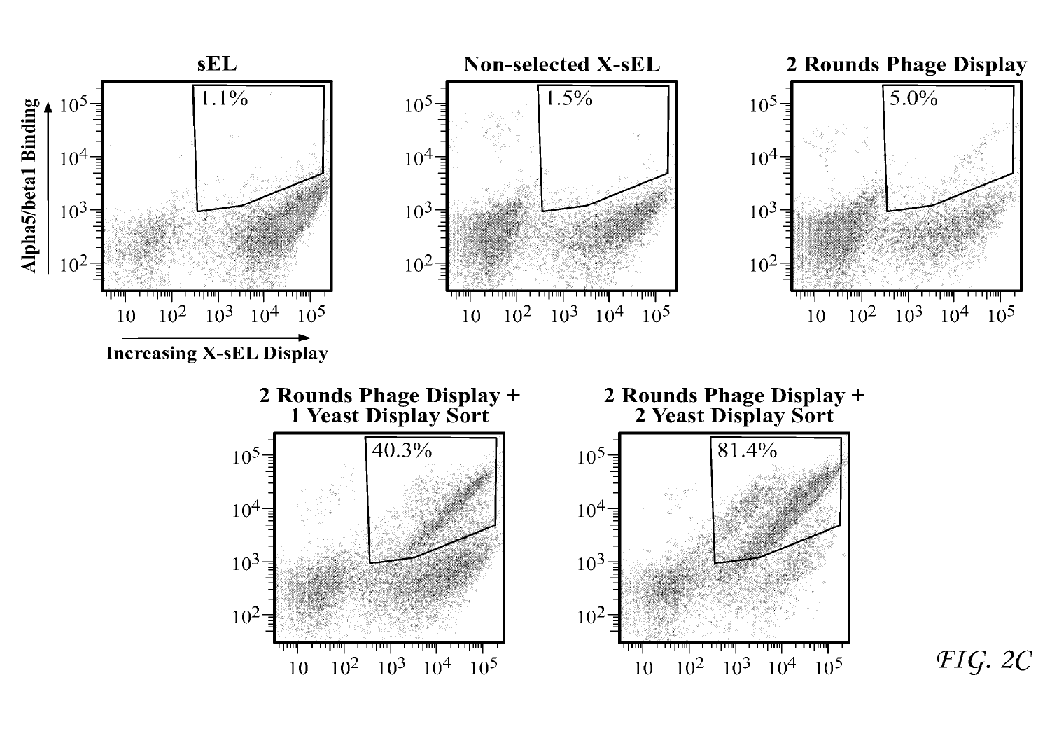

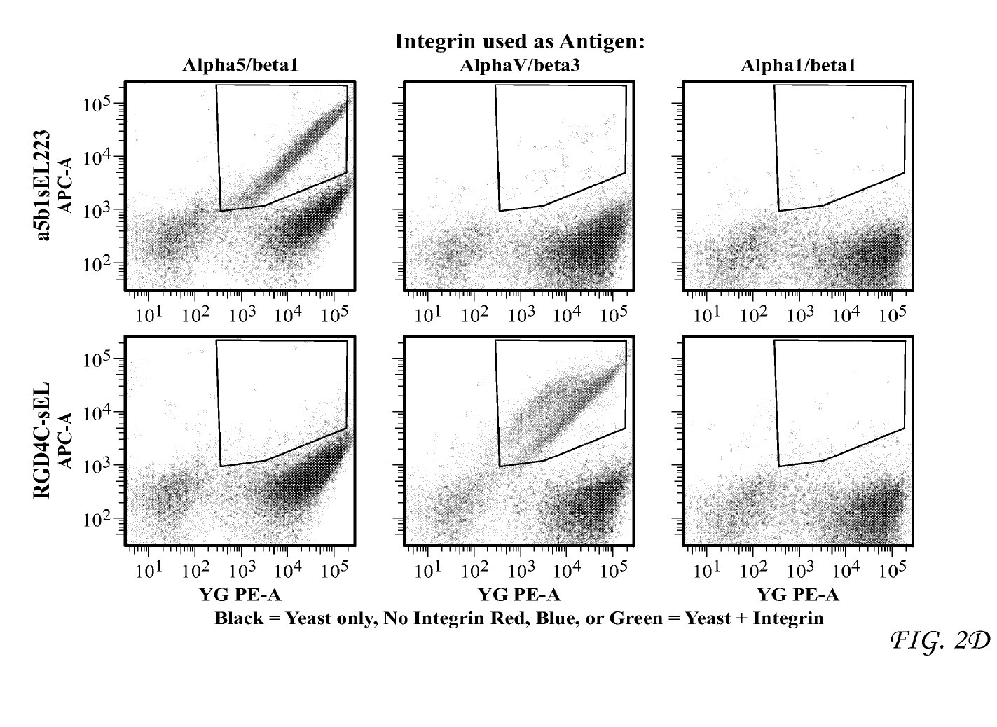

Following 2 rounds of phage display selection and 2 rounds of yeast display sorting, significant enrichment for integrin binding NNK-sEL polymers was observed as shown in FIG. 2C. After the second round of phage panning, >10¹⁰ of the scraped bacteria were miniprepped and used as PCR template for cloning into pDNL7 and yeast. Fragments were amplified in a reaction mix consisting of 10 ul 10× Thermopol buffer, 10 ul 2.5 mM dNTPs, 5 ul 10 uM psrptopdnl7_F (oligo), 5 ul 10 uM psrptopdnl7_R (oligo), 2 ul Taq Polymerase (NEB), 2 ul 10-50 ng/ul template, and 66 ul water. Thermocycling conditions were: 95° C. 1 min, (95 25 sec, 68 50 sec)×25 cycles, then 68 2 min. pDNL7 vector was prepared by digestion with BssHII and NheI (New England Biolabs) and gel extraction of insert and digested vector bands from a 1% agarose gel was performed. S. cereviseae EBY100 cells were grown, made competent and transformed with ˜750 ng cut vector and 10-15 fold molar excess of insert using the Yeast Transformation Kit (Sigma) followed by growth in SD/CAA+tet+kan media for 2-3 days at 30° C. with continuous shaking. Dilutions were plated on SD/CAA agar to assess diversity and transformation efficiency. Recombination was considered to be successful and the library was used if the number of transformants exceeded the input diversity by 1-2 orders of magnitude.

Yeast cultures containing the sEL libraries or known sequences were grown and induced to display as described above. To test binding, biotinylated integrins and yeast were blocked as described above followed by binding on ice. Typically, ˜10⁶ yeast were mixed in a 25-50 ul BB:IPBST solution with integrins at a final concentration of 100-500 nM. Binding proceeded on ice for 1-3 hours with occasional mixing followed by 2-3 washes with IPBST. A secondary solution containing a 1:1000 dilution of mouse-anti-SV5 labeled with Phycoerytherin (PE)+1:250 dilution of streptavidin-Alexa633 (Molecular Probes) in BB:IPBST was used to stain the yeast for 30-60 minutes on ice followed by washes and resuspension in PBS. Individual yeast cells were analyzed and sorted based on display (PE) and binding (Alexa633) of the sEL polymers to integrins using a BD FACSAria Flow Cytometer. In the case of library sorting, cells displaying polymers with significant binding as observed by increase in Alexa633 fluorescence were sorted (see FIG. 2) and grown as indicated above. The process was repeated for subsequent rounds of sorting as necessary. Sorted populations were sequenced using IT-seq (see below) and enriched sequences were cloned and tested as monocultures, along with controls, for binding using the same conditions. Binding data were analyzed and figures were generated using FlowJo (Treestar) or FACS Diva (BD) software.

DNA encoding displayed polymers was extracted and used as template for Ion Torrent sequencing (IT-seq). This unique approach for identifying positive clones departs from traditional methods that involve screening hundreds to thousands of clones derived from the selection and sorting process and instead identify clones based on enrichment of sequences observed in the sequencing output. An IT-seq analysis algorithm (D'Angelo, S. et al. The antibody mining toolbox: An open source tool for the rapid analysis of antibody repertoires. mAbs 6, 41-53 (2013)) was adapted to identify sequences enriched at each round of selection and sorting.

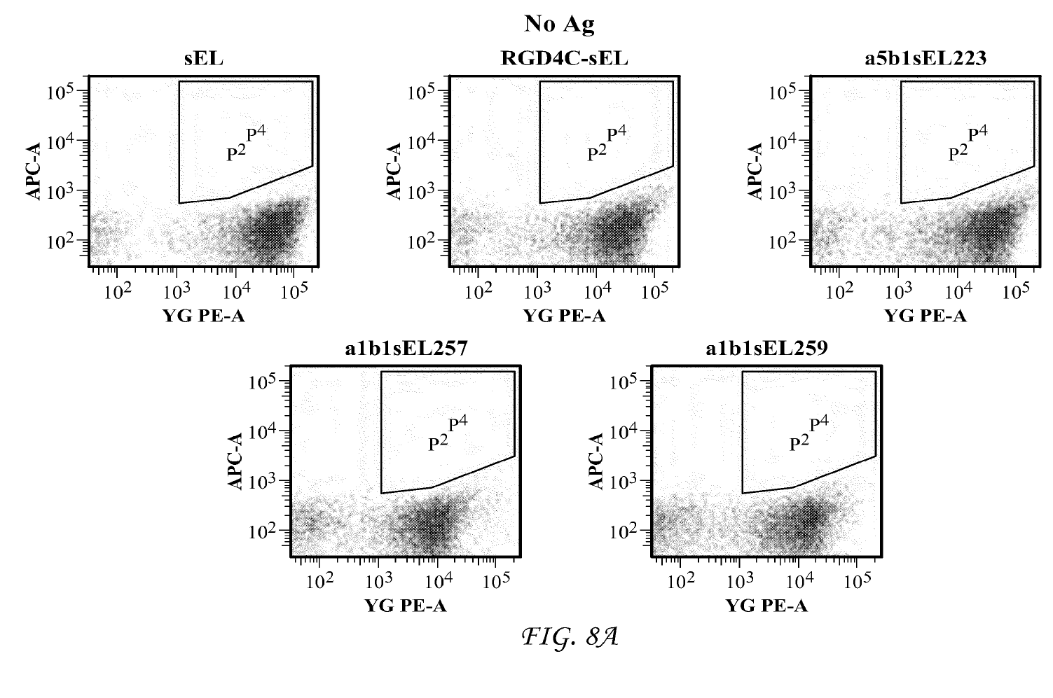

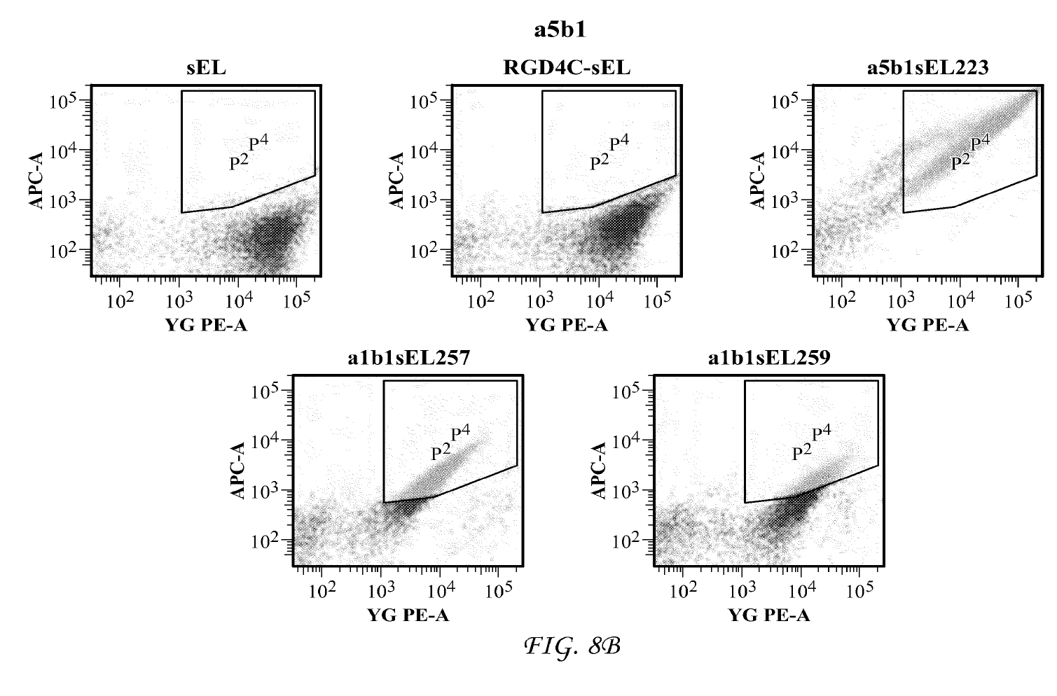

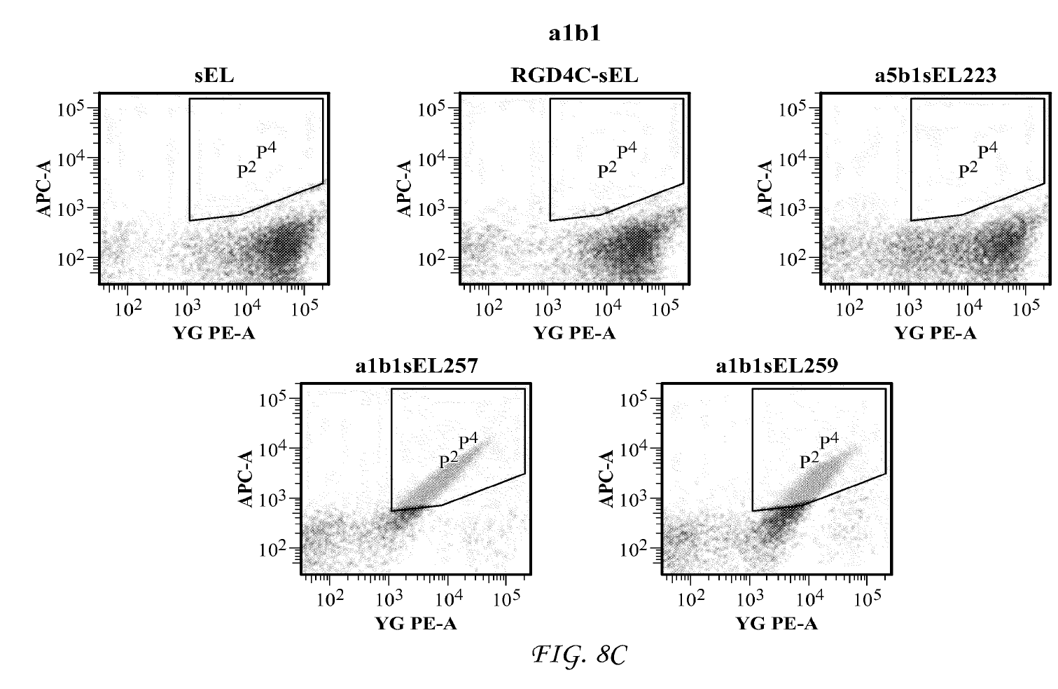

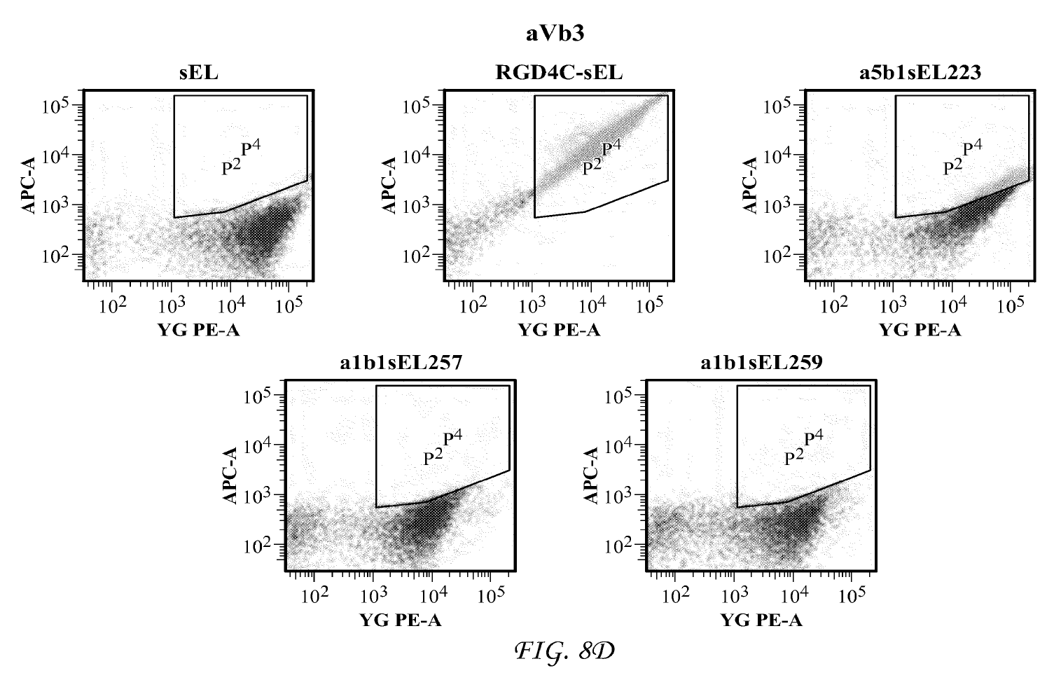

After two rounds of selection followed by two rounds of sorting on yeast, ˜85% of the sequence reads in the sorted pool contained the polymer sequence a5b1sEL223 (See Table 1 and 3). The selected polymer contains a RGDGWL motif flanked by paired cysteines, that is very similar to a sequence discovered in previous phage display selections against the a5b1 integrin. This result demonstrates the functionality of the library; that a recognizable domain could be enriched for from the NS libraries using the disclosed methodology of phage and yeast display followed by sequencing. A subsequent selection and sort against a5b1, using a variation on the helper phage used to package the phage particles used for display (as described in Goletz, S. et al. Selection of large diversities of antiidiotypic antibody fragments by phage display. Journal of Molecular Biology 315, 1087-1097 (2002)), was performed and the sequence of the most highly enriched clone was again shown to be a5b1sEL223, demonstrating the reproducibility of the enrichment for functional binders using multiple phage display packaging systems. Interestingly, although the protein sequence was identical, the DNA sequence varied, demonstrating that the library contains sufficient diversity to select the same protein sequence coded by two different DNA sequences. When this sequence was tested for binding against a1b1 and aVb3 integrins, the polymer appeared to be specific for a5b1 (FIGS. 2D, 8, 9)

TABLE 3
Selected sequences

| Name | Sequence | Antigen | Notes |
|---|---|---|---|
| a5b1sEL223 | AGHCRGDGWL | a5b1 | Does not bind |
| | CTDKGSG[VP | integrin | alb1 or aVb3 |
| | GIG]₂₅* | | integrins when |
| | | | displayed on |
| | | | yeast and tested |
| | | | in flow |
| a1b1sEL257 | AGSARYVWYN | a1b1 | Does not bind |
| | CVPIRIWRGS | integrin | aVb3 but binds |
| | G[VPGIG]₂₅ | | a1b1 and a5b1 |
| | | | when displayed |
| | | | on yeast and |
| | | | tested in flow. |
| a1b1sEL259 | AGHYYGRHWW | a1b1 | Does not bind |
| | LFHVLNYPGS | integrin | aVb3 but binds |
| | G[VPGIG]₂₅ | | a1b1 and a5b1 |
| | | | when displayed |
| | | | on yeast and |
| | | | tested in flow. |
| | | | Does not appear |
| | | | to be specific |
| | | | to a class of |
| | | | integrin |
| mscsEL216 | AGGYYMFSRL | AD- | Binds to MSC |
| | GSG[VPGIG]₂₅ | MSC | cells |
| mscsEL217 | AGGYWHYGQL | AD- | Binds to MSC |
| | GSG[VPGIG]₂₅ | MSC | cells |
| mscsEL218 | AGAPRFRFGT | AD- | Binds to MSC |
| | MYDAGSG[VP | MSC | cells |
| | GIG]₂₅ | | |
| mscsELp46 | AGVVVERKKC | BD- | Binds to MSC |
| | GSG[VPGIG]₂₅ | MSC | cells and |
| | | | promotes |
| | | | spheroid- |
| | | | chondrocyte |
| | | | formation |

*In all cases, the purified protein contains the following sequence C-terminal to sEL: VPASGKPIPNPLLGLDSTHHHHHH (See Table 2)

Having demonstrated the functionality of the sEL library by selecting a recognizable RGD motif, a selection against the a1b1 integrin was performed. The alpha1 subunits varies from the alpha5 subunit in that heterodimers containing alpha1 prefer collagen binding sites and not RGD motifs. Following the same strategy of two rounds of phage display followed by two rounds of yeast sorting, sequences were enriched as monitored by IT-seq. Interestingly, the enrichment wasn't as robust for a single sequence with the top two ranked sequences accounted for ˜4.9% and 3.0% of the total number of sequence reads. The sequences encoding these two polymers, called a1b1sEL257 and a1b1sEL259, were then tested for binding to integrins a1b1, a5b1, and aVb3 to verify binding and assess specificity. The polymers bound both b1 containing integrins and did not bind aVb3, implying selected polymers with specificity towards b1 integrins (FIG. 2D, 8, 9). Further testing revealed binding of these selected polymers to the human aLb2 integrin, demonstrating binding to multiple classes of integrins. This demonstrates that RGD-containing, non-RGD containing, and polymers with varying degrees of specificity can be selected from sEL libraries and that combining phage panning, yeast display, and next generation sequencing provides a unique process for identification of novel polymers.

### MSC Selections

The naïve polymer libraries were also selected for binding to MSC derived from either adipose tissue (AD-MSC) or bone marrow (BD-MSC). Three to five rounds of phage display panning were performed, followed by IT-seq of the outputs from each round of selection (FIG. 2A-B), as described below.

Selections were performed against either Adipose-derived (AD) or Bone-derived (BD) MSC cells. MSCs and phage were prepared as described above and prior to selections, cells were blocked in MSC media+2% BSA and phage in BB on ice for 20-60 minutes. Cells and phage were mixed (˜5×10⁶ MSC cells+10¹¹ phage in 750 ul) with rotation at 4° C. for 1-2 hours. The mixture was spun down (˜2000×g 30 sec) and washed with PBST followed by 3 washes with PBS. The number of PBST washes increased with increasing rounds of selection from 5 in the first round to 10 in the final round. For AD-MSC selections, bound phage were eluted by addition of 50 ul of M-PER lysis reagent (Thermo) and incubation at 37° C. for 20-30 min. BD-MSC bound phages were eluted by addition of 0.1N HCl followed by neutralization with 1.5M Tris pH 8.8. In both selections, phage were produced using KM13 helper phage which enables proteolysis of non-displaying phage such that only functional phage are propagated to the next round, as described in Goletz, S. et al. Selection of large diversities of antiidiotypic antibody fragments by phage display. Journal of Molecular Biology 315, 1087-1097 (2002). Proteolysis was accomplished by diluting the elutions 10-fold in PBS and adding Trypsin to ˜1 mg/ml followed by infection of OD 0.5 DH5αF′ for 30 min at 37° C. Bacteria were then plated on amp/glu agar plates and with subsequent steps as described above. Five and three rounds of selection were performed against AD-MSC and BD-MSC cells (respectively) with output phage titers increasing by an order of magnitude with each round.

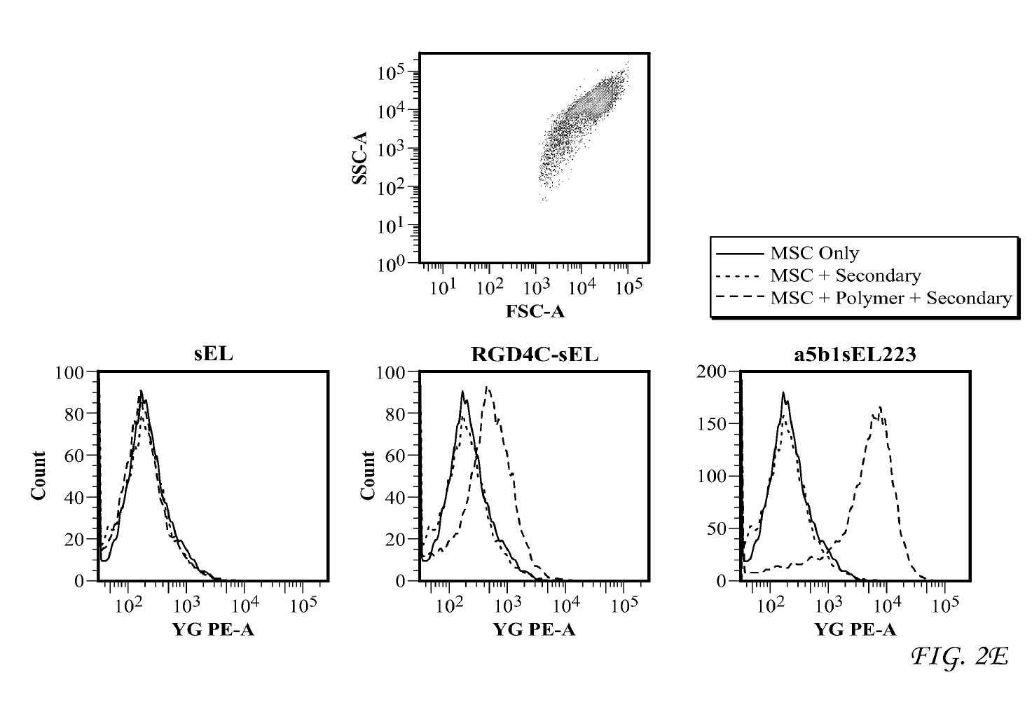

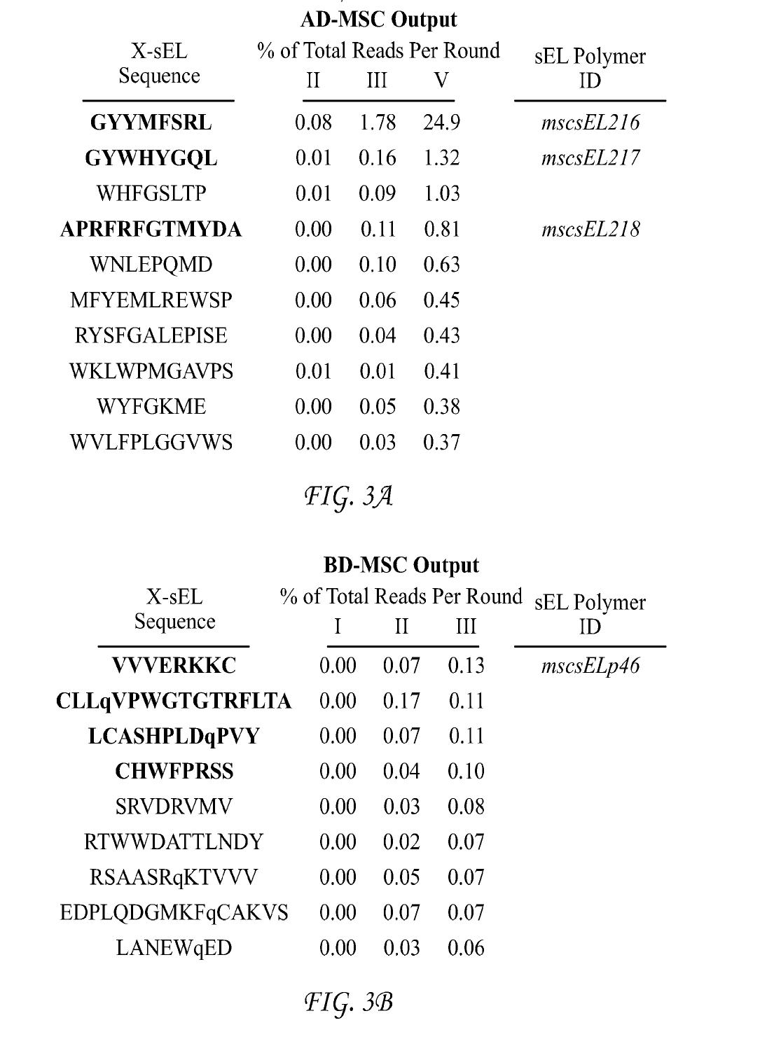

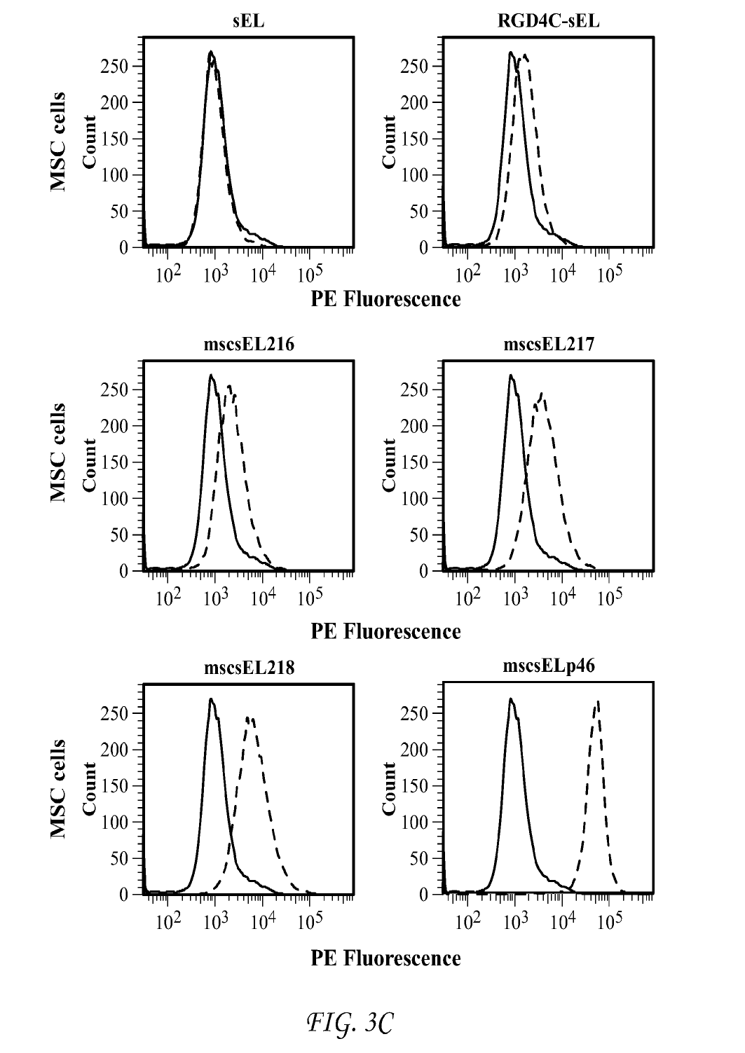

Selections against AD-MSC resulted in enrichment of several sequences, with the top ranked one accounting for ˜25% of the total reads and the remaining accounting for 0.4-1.3% of the total reads (FIG. 3). Selections against BD-MSCs resulted in lower levels of enrichment, with the top five most highly ranked sequences accounting for ˜0.1% of the total reads. The use of deep sequencing proved to be indispensable to detect enrichment during these selections, especially when fewer rounds of panning were performed, as in the BD-MSCs selection. Thousands of clones would have needed to be screened, one by one, using traditional methods, with no guarantee of success. It is therefore not surprising that the sequences identified did not contain any previously described binding motif Another possible explanation for this result could be that previous selection of cell binding motifs never used libraries of peptides within the sEL context. The highly ranked clones were tested for binding to cells, as purified polymers.

### Ion Torrent Sequencing

Bacteria from phage panning outputs or sorted yeast were miniprepped (Qiagen) to isolate plasmids to be used as template in PCR to generate X-sEL amplicons for Ion Torrent Sequencing (IT-seq). Oligos used for PCR are listed in Table 2 and conditions for PCR are as follows: 10 ul 5×GC buffer (1×), 2.5 ul DMSO, 5 ul 2.5 mM dNTPs (250 uM each), 2.5 ul of each 10 uM oligo (0.5 uM each), 2 ul 20-100 ng/ul sEL-pSRP template, 0.5 ul 2 U/ul Phusion HF Polymerase, and 25 ul of water with thermocycling conditions of 98° C. for 30 sec, 30 cycles of 98° C. 10 sec, 65° C. 10 sec, 72° C. 15 sec, and a final extension of 72° C. for 3 min. Each output was amplified with an Ion MID R oligo containing unique barcode MID sequence. The ˜250 bp amplicons were gel purified and quantified using the Qubit dsDNA quantification assay (Life Technologies). The Ion Xpress Amplicon library protocol was used to prepare the sample for sequencing on the Ion 316 chips (Life Technologies). Sequences were quality filtered, binned by MID barcode, and analyzed using the AbMining Toolbox, as described in D'Angelo, S. et al. The antibody mining toolbox: An open source tool for the rapid analysis of antibody repertoires. mAbs 6, 41-53 (2013), adapted to identify variable regions of the sEL polymer sequences. The naïve, or non-selected (NS) libraries, were pooled and sequenced to assess amino acid abundance and length of the NNK diversity. Amino acid abundance and length matched theoretical expectation (FIG. 2B).

### Protein Production and Purification

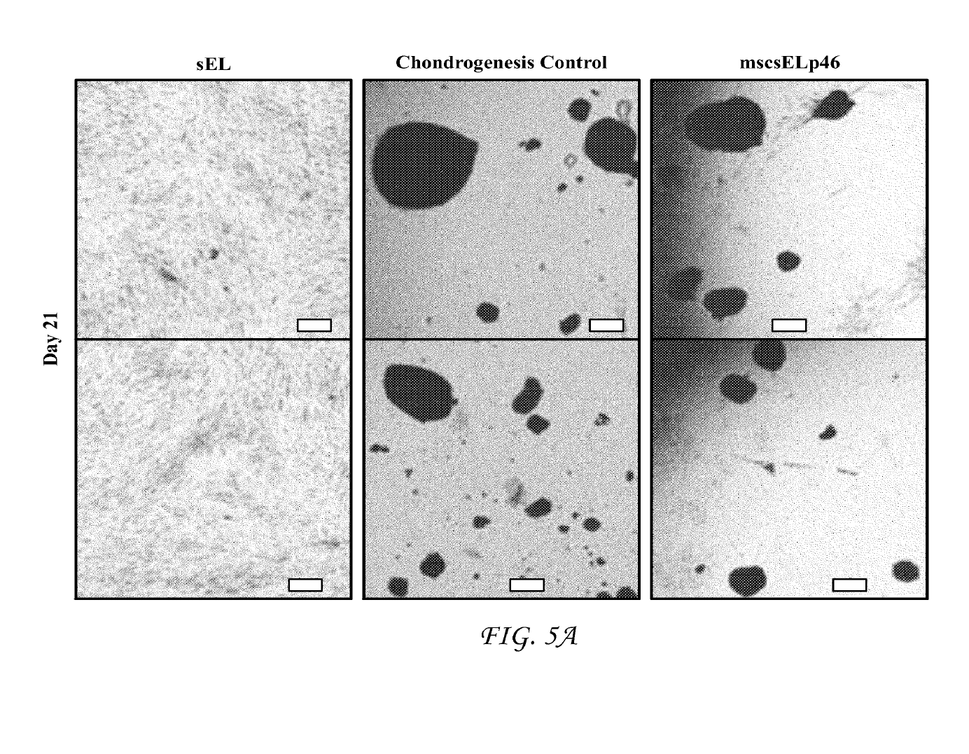

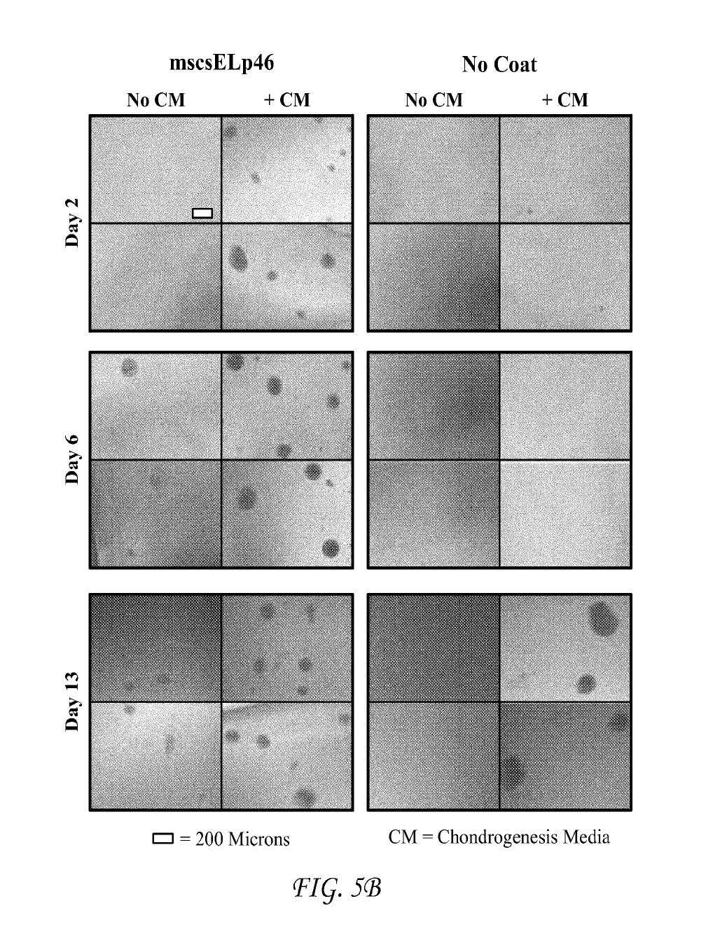

Control and down-selected sequences identified in selections and sorting were cloned into the POE vector for protein expression. The POE vector used for protein expression appends a pelB leader to the N-terminus of the expressed protein that is cleaved upon secretion into the periplasmic space. Proper cleavage of the leader sequence was predicted by the SignalP server (http://www.cbs.dtu.dk/services/SignalP/) and verified by mass spectrometry. Expression from the POE vector also provided C-terminal SV5 and His₆ₓ tags for detection. sEL expression and purification protocols were based on the strategy described in Hassouneh, W. et al. Unexpected Multivalent Display of Proteins by Temperature Triggered Self-Assembly of Elastin-like Polypeptide Block Copolymers. Biomacromolecules 13, 1598-1605 (2012). Briefly, plasmids were transformed into electrocompetent BL21(DE3) E. coli cells and plated on selective 2xyT+amp solid agar medium. Liter flasks of sterile 2xyT+amp were inoculated with 15 ml starter cultures grown from multiple colonies of fresh transformants and allowed to grow shaking at 250 rpm for 24 hours at 37° C. uninduced. It was determined that leaky expression from the vector's T7 promoter yielded 20-100 mg/L soluble protein, depending on the construct, and this yield was not significantly improved by IPTG induction. After 24 hours, cells were harvested by centrifugation (20 min 4000 rpm 4° C.) and cell pellets were either processed immediately or stored at −80° C. The periplasmic fraction was isolated by osmotic shock. Cell pellets were resuspended in 80 ml/L culture cold 20% sucrose and incubated on ice for 15 minutes. Resuspensions were centrifuged for 10 min at 6000 rpm 4° C. and the supernatant saved as periplasmic fraction 1. Cell pellets were then resuspended in cold nanopure H₂O and incubated and centrifuged as above. This supernatant was saved as periplasmic fraction 2 and pooled with fraction 1. sELs were purified by subjecting the pooled periplasmic fractions to iterative inverse temperature cycling for 2-4 cycles with the “hot” phase separation facilitated by the addition of dry NaCl to a final concentration of 3M for the first precipitation and the addition of 5M NaCl as needed for subsequent cycles. Protein purity and size was assessed using SDS-PAGE followed by Coomassie-based stain (GelCode Blue, Pierce; FIG. 5). Purified sELs were dialyzed into nanopure H₂O overnight at 4° C. and lyophilized. After quantification by weight, protein was either stored at −20° C. or resuspended in water, sterile filtered, aliquoted, and stored at −80° C. Protein concentrations used throughout were determined based on dry weight mixed in a given volume of water.

### sEL Protein Binding to MSC Cells

MSC cells were detached, washed and blocked as described above. Purified sEL proteins were added to ˜10⁶ blocked MSC cells in BB:IPBST buffer at ˜30 uM in a final volume of ˜100 ul and bound on ice for 1-2 hrs with occasional mixing by pipetting. Samples were washed 3 times with 1 ml of BB:IPBST by spinning at 2000×g for 2 min, then stained with 1:1000 dilution of mouse-anti-SV5-PE for 20-30 minutes on ice. Washes were repeated and analyzed on a BD FACSAria flow cytometer and generated figures using FlowJo software (Tree Star).

### MSC Differentiation and Growth on Deposited sEL Polymers

Various sEL proteins (filter sterilized) were resuspended to 1 mg/ml then 150 ul drop cast onto 1 cm² borosillicate chamber slides (Lab-Tek) and coated for 16-24 hours at 4° C. Excess liquid was removed and slides were allowed to evaporate for an additional 30 minutes under a sterile hood (as additional precaution, chamber slides can be UV treated at 3.6 KJ/m² without alteration of protein function). 8000-10000 AD-MSC cells were seeded onto each well in 400 μL MSC media, and grown under standard conditions (see above). Control cells (e.g. without polymer) were subjected to differentiation by replacing the MSC media with chondrogenesis differentiation media (GIBCO), 24 hrs from seeding. Cells were monitored continuously for up to 28 days following seeding with media changes every 2-4 days. Cell aggregates were stained with an antibody to assess Aggrecan expression and Safranin O to stain proteoglycans, both hallmarks of chondrogenesis.

### Selecting Cellular Adhesive and Responsive Polymers

Polymers with affinity (cell-adhesive) for each cell type (e.g. osteoblasts (Promocell Inc.) or primary bone marrow stromal cells (Tulane/NIH Center) will be selected using techniques previously established for the selection of peptides and antibodies, using either phage or yeast display. Briefly, cells will be grown on solid substrates. Phage or yeast displayed polymer libraries will be interacted with the plated cells, at different temperatures. Nonspecific polymer-phage/yeast will be washed away, and specifically bound polymers eluted by heat, acid, light or competition with extracellular proteins or solubilized cells. After 2-4 selection rounds, outputs will be recloned into expression vectors and screened, as detailed above. As an example, individual polymer clones expressed in 96-well deep-plates and initially purified by temperature precipitation. Cells will be seeded onto tissue culture plates, previously coated with selected polymers, and cell morphology and growth will be monitored by phase contrast- and/or fluorescence staining and microplate imaging/reading to identify biocompatible adhesive polymers. Cell specificity will be assessed by testing the ability to support the growth of other cells. Proliferation and additional differentiation will be monitored by specific cellular assays (i.e. osteocalcin, BMP-2, hydroxy apatite, etc) and microscopy. These polymers will be further downselected for those that release the cells based on addition of temperature, light, or as a function of cellular growth. Libraries will be analyzed before and after selection using deep DNA sequencing, in order to identify sequence motifs that are advantageous (frequently selected) and deleterious (rarely selected), which will allow better design (predict) of subsequent polymer scaffolds.

### Selecting Optically and Thermally Responsive Polymers for Optical Electronics

To select optically and/or thermally responsive polymers, light and/or heat will be applied to yeast-displayed or phage-displayed (within microemulsions) polymers and sorted/selected by flow cytometry. The heat stimulated polymer libraries will be excited by laser wavelengths available on the FacsAria (405, 488, and 633 nm), and analyzed and sorted for fluorescence at different wavelengths utilizing the full spectrum of available emission filters. The polymers will then be further downselected for those that release cargo (polymers assembled in presence of a fluorophore, dialysis, and monitoring the release of the fluorophore) as a function of temperature or light excitation. The selection is intended for targets in both molecular electronics and biomaterials, for example, polymers that are electrochemically active for differentiating cells (e.g. neurons) and biopolymers with ability to follow cellularly-initiated polymer degradation via changes in fluorescence.

### Polymer Analysis and Characterization

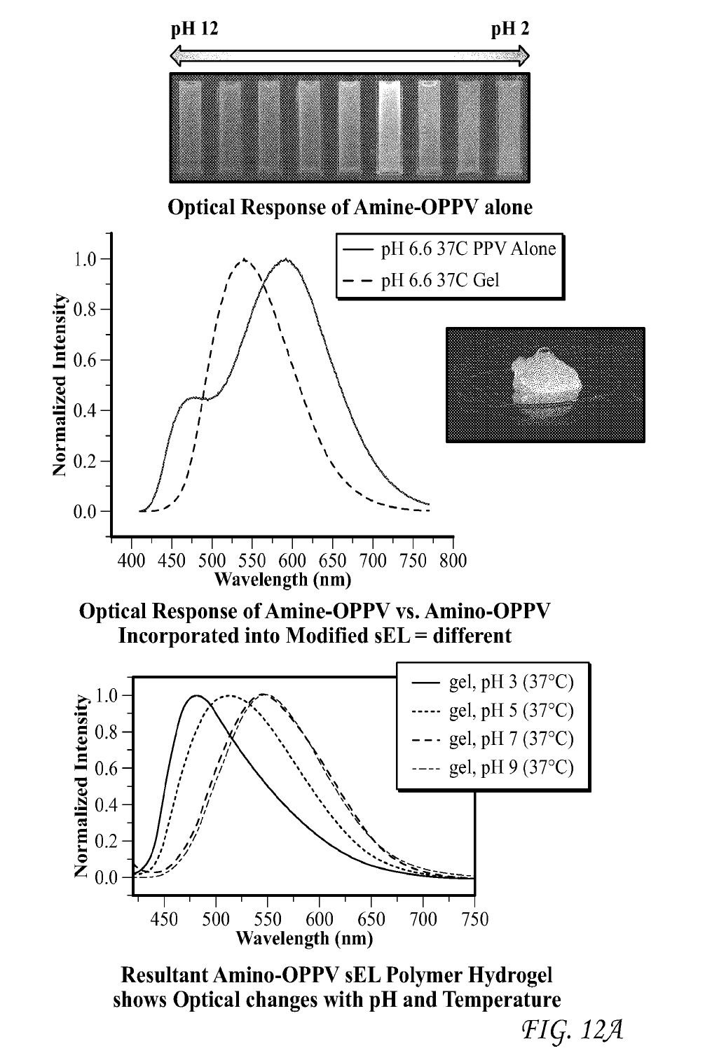

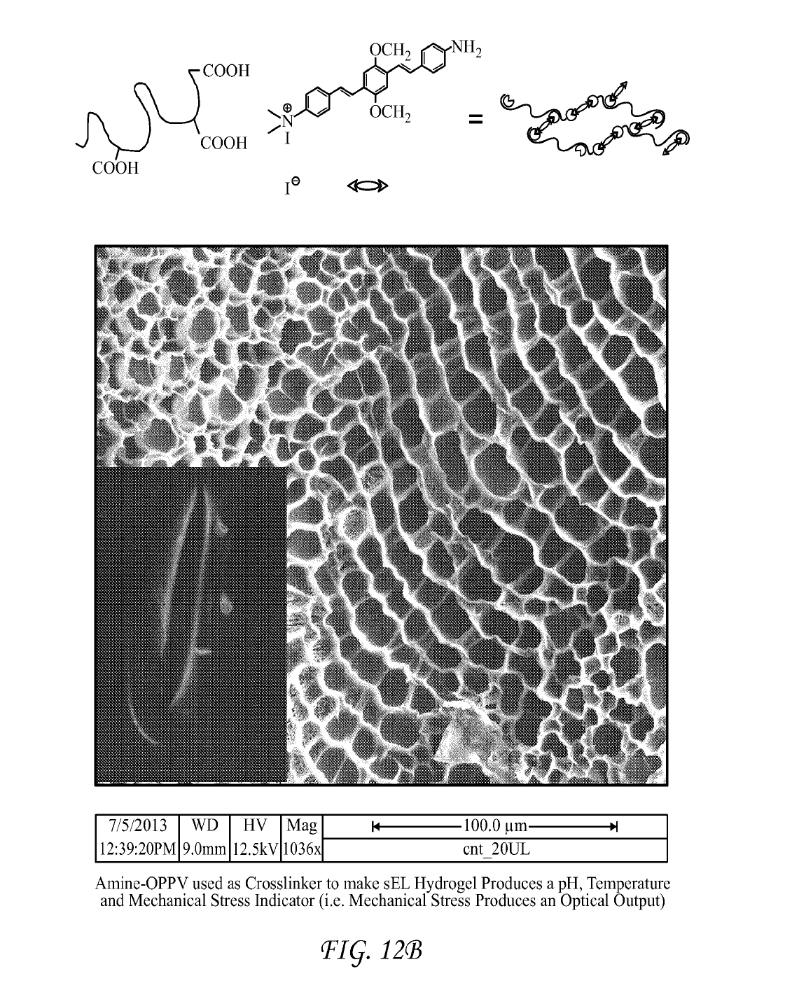

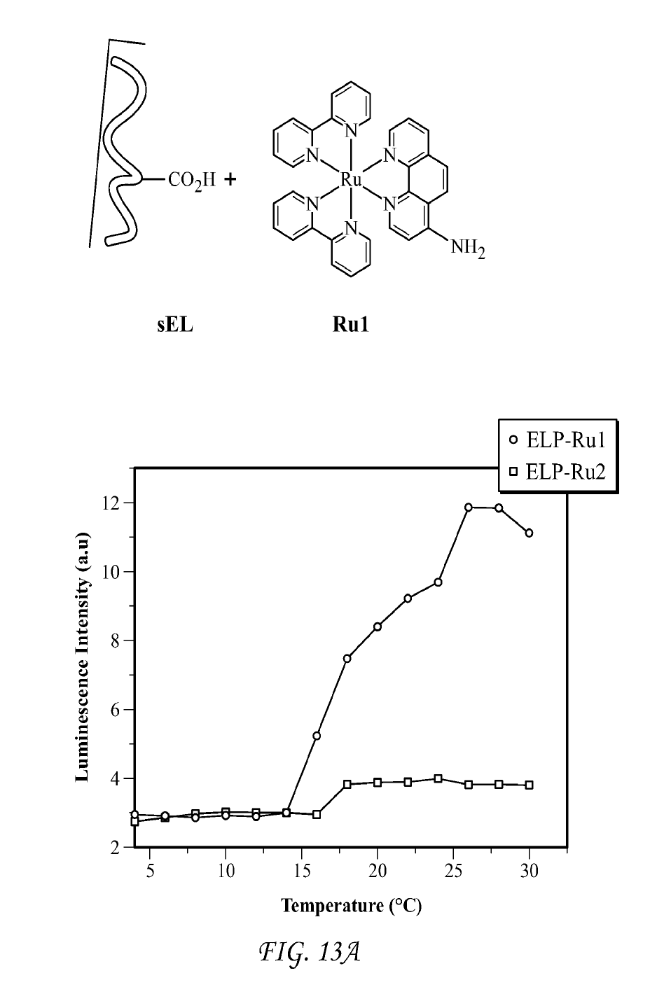

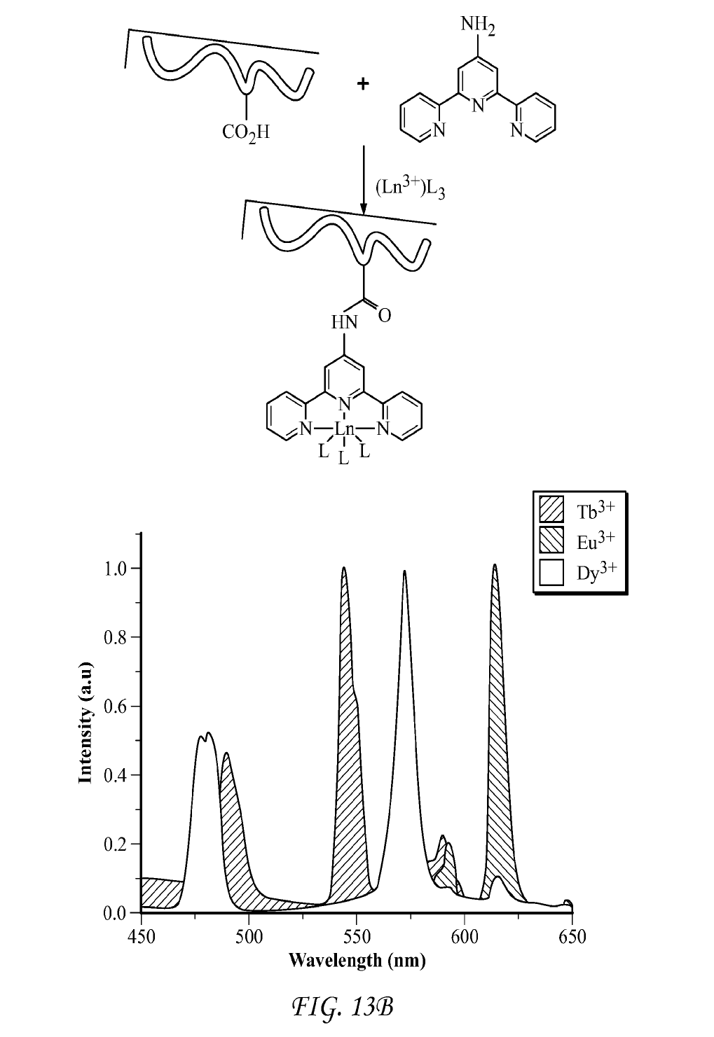

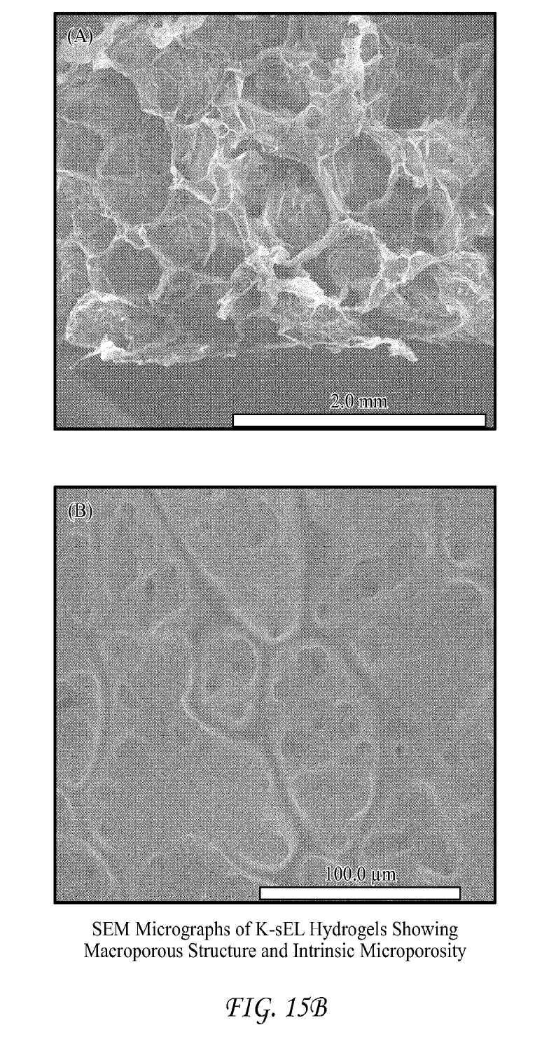

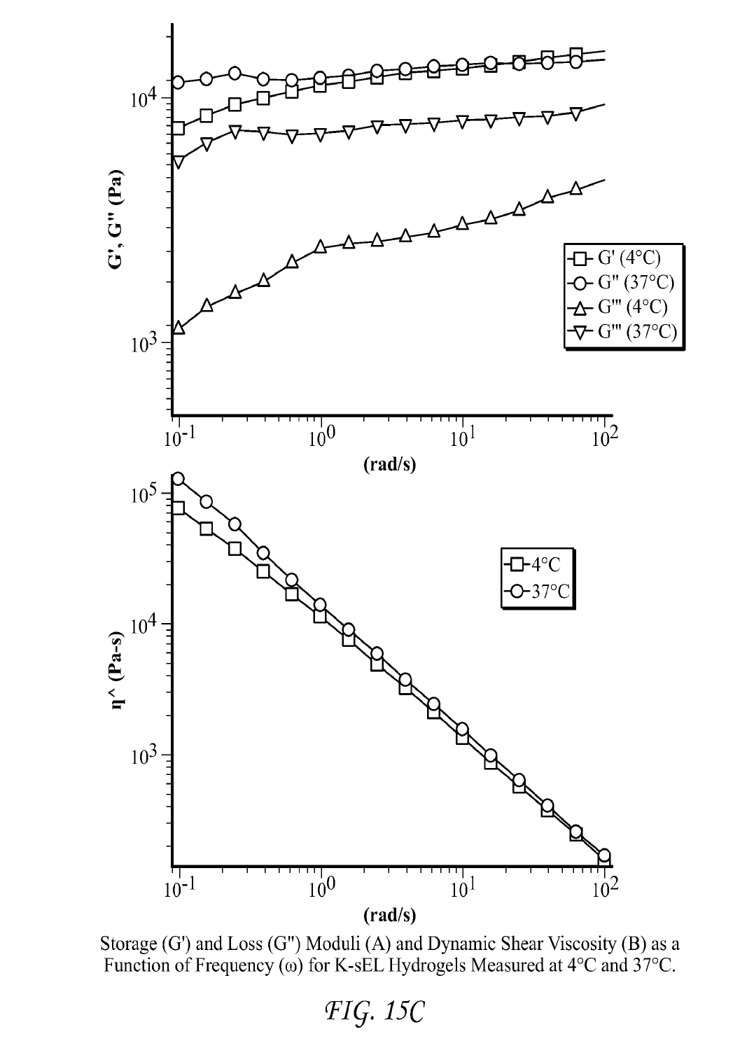

Beyond the cellular studies, the properties of down-selected and purified protein-polymers can be studied with a variety of standard instruments and techniques of routine application. Contact angle (hydrophilicity) can be measured using a Tantec CAM plus microcontact angle meter. Tensile strength, Young's modulus and mechanical stiffness can be measured as a function of strain and temperature (23-65° C.). The phase transition on temperature and pH changes can be monitored by turbidity, dynamic light scattering, fluorescence correlation spectroscopy and microscopy (AFM and TEM), where appropriate. UV-Vis can be used to monitor the amount of thiophene on the polymer (on/off phage). Optical properties (absorption/emission) as a function of redox potentials can be studied using an in situ combination of electrochemistry with UV/Vis/near-IR and fluorescence spectroscopies. An exemplary characterization (FIG. 15; note that FIGS. 12 and 13 also demonstrate characterization) is demonstrated for the production and characterization of the sEL utilized for the OPPV-amine sEL (K-SEL) hydrogel demonstrated in FIG. 12A.

The K-sEL gene was designed based on the ELP-1 construct described previously. To add an amine group to facilitate cross-linking, the N-terminal sequence AGKGS was introduced using a PCR primer to amplify ELP-1. Purified PCR product was subcloned into the BsshII and NheI sites of the POE expression vector, which contributed C-terminal SV5 and 6×His tags and a leader sequence that is removed upon protein secretion to the periplasm. Successful clones were verified by sequencing (MWG Operon).

K-sEL was expressed from BL21(DE3) E. coli cells without induction using the leaky T7 promoter. Typically, 1 L SuperBroth (MP Biomedicals) supplemented with 100 μg/mL carbenicillin was inoculated with 15 mL of overnight culture grown from freshly transformed colonies. Following cell harvesting by centrifugation, K-sEL was released from the periplasm via cold osmotic shock (20% sucrose/1×PBS) and purified as described elsewhere. ELP purity was verified by SDS-PAGE. Dynamic light scattering (DLS) experiments to study temperature-dependent coacervation were performed on a Zetasizer NanoZS (Malvern). Three volume measurements of 10 mg/mL K-sEL were acquired at 2° C. intervals from 4° C. to 40° C. with two-minute equilibration times at each temperature. The average hydrodynamic diameter was plotted±standard deviation (error bars).

### Preparation and Characterization of ELP Hydrogels

Hydrogels were generated by dissolving lyophilized K-sEL at a concentration of 106.7 mg/mL in 85% DMSO:15% DMF. The trifunctional crosslinker tris-succinimidyl aminotriacetate (TSAT) was added dry to a final concentration of 3.7 mg/mL and the solution was vortexed immediately and quickly pipetted (˜100 μL per gel) into Eppendorf cap molds. Dry TSAT stored at 4° C. was found to be more stable than resuspended aliquots of TSAT in DMSO/DMF stored at −80° C. While the solution became too viscous to pipette within a few minutes, gelation was allowed to continue overnight undisturbed before gels were removed by shrinking with 1 mL of 5M NaCl for several hours.

To determine the insoluble (gel) fraction of the hydrogels, three 100 μL hydrogels (10.67 mg of polymer) were weighed after lyophilization following extraction of the soluble polymer fraction by immersion in 10 mL of water for 48 h. The insoluble fraction was determined by the formula gel fraction (hydrogel %)=(W_{d}/W)*100 (where Wᵢ is the initial weight of the polymer in the sample (10.67 mg) and W_{d} is the weight of the insoluble fraction after extraction and drying). The reported solubility percentage is the average±standard deviation of three measurements; the weight contribution of the cross-linker was ignored. The degree of swelling was calculated as follows: swelling=(Wₛ−W_{d})/W_{d} (where Wₛ is the weight of the hydrogel in its swollen state after removal of the soluble fraction and W_{d} is the weight of that same hydrogel following lyophilization). The reported ratio is the average±standard deviation of three gels.

### Scanning Electron Microscopy (SEM)

SEM micrographs were obtained on an FEI Quanta 400 FEG-E-SEM environmental microscope (resolution 3-4 nm, high voltage range from 500V-30 kV) after sputter coating lyophilized hydrogel samples with 1 nm gold. SEM images were collected from a range of voltage spanning from 12.5-20 kV.

### Rheology

Rheological data were obtained on a TA Instruments Advanced Rheometric Expansion System (ARES) rheometer equipped with forced air convection environmental chamber and parallel plate geometry (8 mm diameter). Though applied shear strain is known to vary under parallel plate configuration, plate radius was small enough to assume the applied shear strain gradient was insignificant. Hydrogel discs were tested under 1 mm (4° C. and 37° C.) and 2 mm (25° C.) gaps. Gap height was adjusted for hydrogel shrinkage due to rapid water loss in non-ambient environmental chamber conditions. A dynamic oscillatory strain sweep was performed at 25° C. across a range of 0.1-10% strain at a frequency of 1 rad/s. Dynamic oscillatory frequency sweeps from 0.1 to 100 rad/s were performed at 5% percent strain at both 4° C. and 37° C.

Hydrogels were placed directly in cuvettes against a moveable piece of quartz for vertical support. Temperature control was achieved using temperature-controlled cuvette holders (Quantum Northwest). Absorbance measurements were made on a Varian Cary 300 Bio UV-visible spectrophotometer (at 1.0 nm resolution) and a small-volume sample cells (150 μL) with a 1.0 cm path length. Fluorescence measurements were obtained on a Horiba Jobin Yvon Fluoromax-4 spectrofluorometer and on a Varian Cary Eclipse fluorescence spectrometer. In lifetime measurements, the spectrofluorometer was coupled with a time-correlated single photon counting (TCSPC) system from Horiba Jobin Yvon. The apparatus was equipped with a pulsed laser diode source (NanoLED) operating at 1 MHz and with excitation centered at 390 nm. Each measurement was terminated when a maximum peak preset of 20,000 photon counts was reached for the monitored fluorescence. Analysis of fluorescence decay profiles was performed with the Horiba DAS6 software.

Dynamic light scattering was performed on a Malvern NanoZetasizer.

### Polymer Design and Evolution

A conceptual distinction between general proteins and polymers is that the interactions in general proteins can be unique for each residue and therefore modeling requires considering arbitrary sequence perturbations, whereas for polymers only a reduced set of replicated interaction patterns are necessary, making prediction of polymer structure achievable. Functionally-selected polymers will be analyzed to better design new libraries and new polymer scaffolds with predicted properties will be designed. Useful parameterizations of the protein sequence space will be defined and related to both prediction of interaction, selection, and modeling observations; ultimately, enabling predictive design of materials with specific functions.

For short ELPs, for example, MSL software will be modified to include canonical beta-strand interactions. By restricting the template and variable positions the variation both computationally and in polymer libraries can be usefully exhausted. Additionally, the block-assembly can be hierarchically confined by using macro-elements. These macro blocks present consistent inter-block interfaces that can be decorated with orthogonal conjugate patterns (e.g. polarity, salt-bridges and large-small residue alternation) to define the docking interactions. Four-helix bundles form a compact domain presenting regular surfaces for simplified quaternary structure tiling into open or closed symmetries.

These have at least three advantages: 1) due to the strength of the scaffold, the loop parameterization can be decoupled from the packing interface parameterization; 2) they have a defined hydrophobic interior pocket that we have previously demonstrated to be highly tolerant of invasive functionalization; and 3) inter-bundle strand swapping may be used as a design element in non-planar structures.

### Generation of sEL Libraries

The goal was to generate complex, ELP-based libraries that consist of sequence blocks conferring unique adhesive and transitive properties. To demonstrate that libraries with very large diversities based on ELPs could be generated and used for selections, different libraries with diversity consisting of 8, 12, or 16 random amino acids on the N-terminus of sEL were generated. Each amino acid is encoded by the DNA sequence NNK resulting in at least one codon for each amino acid. Non-selected (NS) Libraries were generated using the circular polymerase extension cloning (CPEC) as previously described (Quan, J. & Tian, J. Circular polymerase extension cloning for high-throughput cloning of complex and combinatorial DNA libraries. Nature Protocols 6, 242-251 (2011), resulting in a combined diversity of ˜2.5×10⁸. CPEC proved to be a far superior method for making highly diverse libraries while using much less DNA than conventional restriction digest followed by ligation (data not shown). To determine if the display platform drives sequence bias for NS libraries, homologous recombination was used to subclone the library into a yeast cell strain optimized for display and the amino acid composition bias of the NS libraries was assessed using Ion Torrent sequencing and an adapted version of our previously described analysis tool, described in D'Angelo, S. et al. The antibody mining toolbox: An open source tool for the rapid analysis of antibody repertoires. mAbs 6, 41-53 (2013). Three versions of the NS libraries were sequenced: 1) the primary library generated from phage that had undergone a single infection into DH5alpha cells, 2) subcloned NS libraries yeast that were sorted based solely on morphology, and 3) the same subcloned NS library in yeast, but only those yeast displaying sEL clones from the library based on an antibody recognizing the SV5 epitope (see FIG. 1). When comparing the frequency of any given amino acid to the frequency for that amino acid expected based on the theoretical composition in NNK libraries, it was found that the NS libraries all consisted of very similar amino acid frequencies (FIG. 1B). Further, the composition of displayed polymers on yeast matched the total yeast population demonstrating that there is little observable bias for sequence when displayed yeast.

Ashort elastin library containing a highly repetitive gene (elastin amino acids: (VPGIG)₂₅; synthetic gene: GenScript) and a six amino acid random peptide insert was created, providing the diversity to select for cell-adhesive polymers. Two types of libraries were created: fully random (NNK, at the DNA level) or constrained amino acids (Y,G,S). Further, a long elastin library was created with three inserts of random peptides (kunkel mutagenesis). These libraries were easily displayed on the surface of phage (protein-3 of phage and aga-2 of yeast are amendable to display the M.W. of the proposed polymers), without recombination, and the libraries could be made in high diversity (˜10⁵⁻⁷).

### Cell-Targeted Selections

In parallel phage display selections were performed against Adipose- or Bone-derived MSCs (AD-MSC or BD-MSC). Three to five rounds of phage display were performed followed by IT-seq of the outputs from each round of selection (FIG. 3). Selections resulted in enrichment of sequences with the top ranked sequence accounted for ˜25% of the total reads with 5 other sequences each accounting for 1-2% of the total reads (FIG. 3). Selections against BD-MSCs resulted in lower levels of enrichment, with the top five ranked sequences accounting for ˜0.1% of the total reads. The sequences identified did not contain known binding motifs showing that selections with sEL libraries can identify sequences that are not currently known. Although the level of enrichment varied, the highly ranked clones were tested for binding to cells as purified polymers.

### Cell Binding Using Purified Polymers

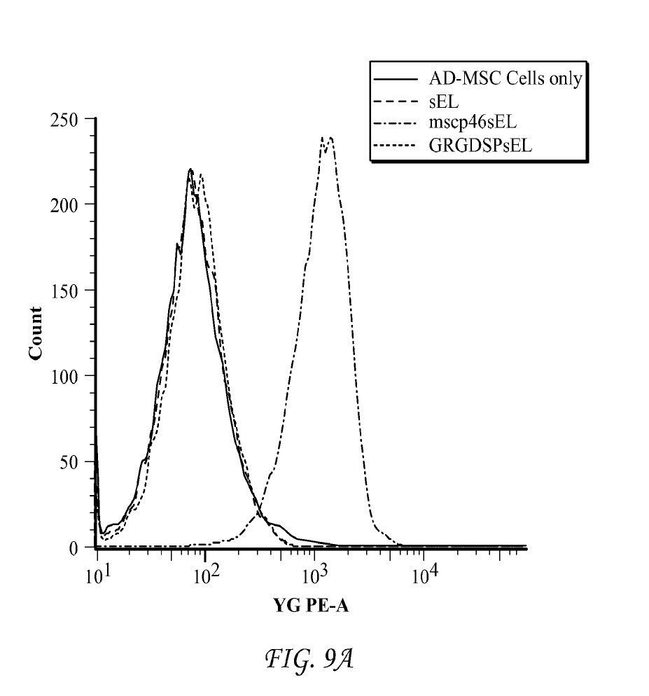

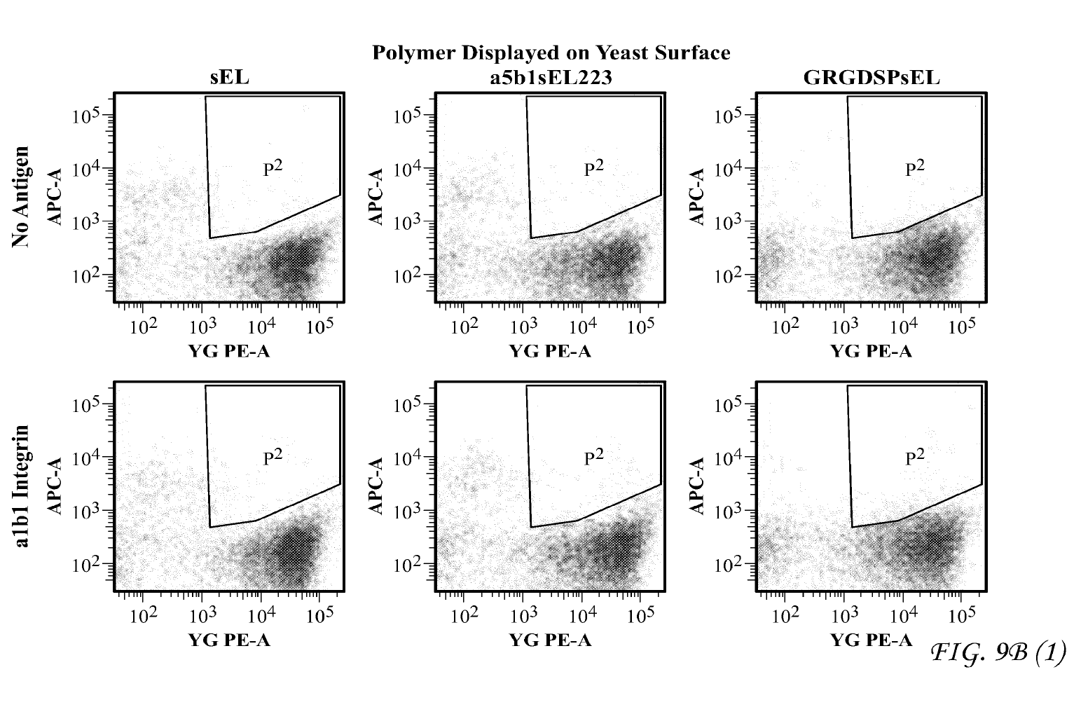

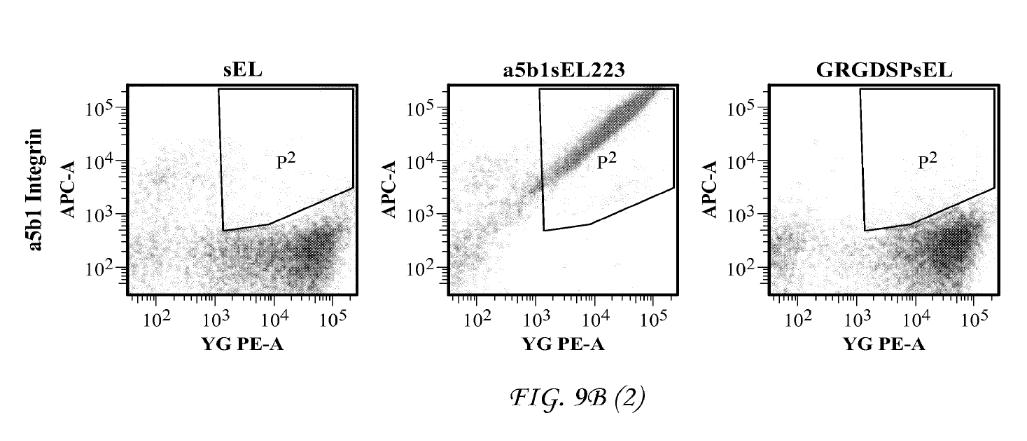

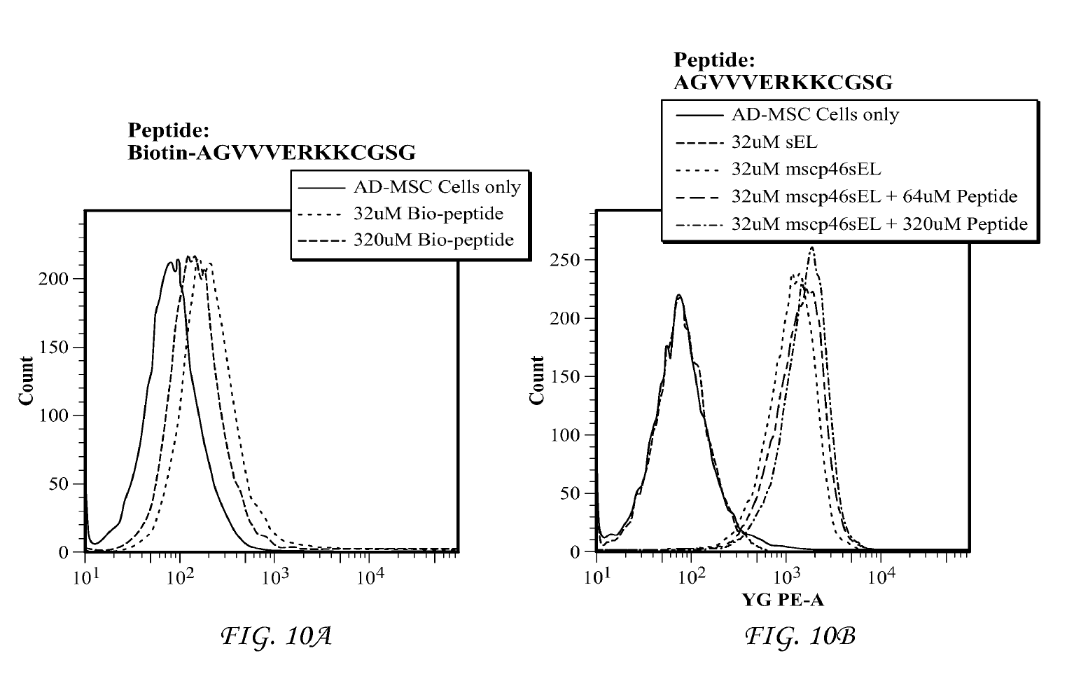

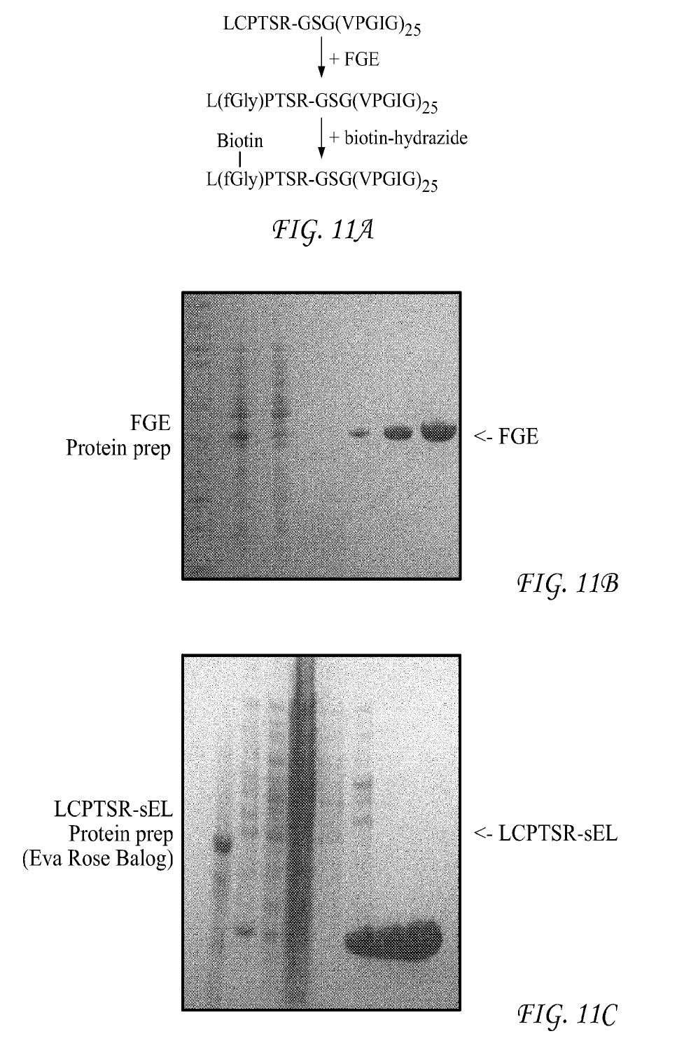

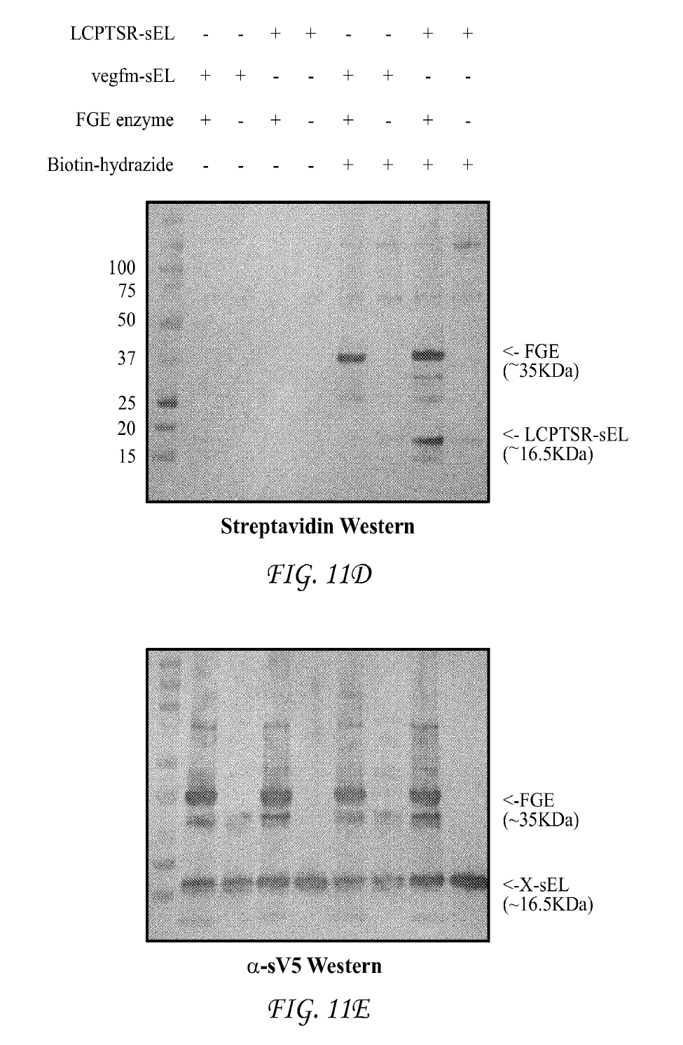

Sequences identified using IT-seq enrichment against MSCs and integrins were synthesized, expressed, and purified from E. coli. The purified polymers were tested for binding to AD-MSCs in flow cytometry to assess whether identification by IT-seq enrichment is a viable method for identifying functional polymers. The majority of down-selected polymers bound to AD-MSCs to varying degrees (FIG. 3). All three sequences (mscsEL216, 217, and 218) tested that came from the AD-MSC selections bound cells in this assay and one (mscsELp46) out of the top four ranked sequences tested from BD-MSC selections bound MSCs in this assay. In addition, the a5b1sEL223 polymer demonstrated binding against MSCs in the same assay, demonstrating that a5b1sEL223 binds as purified polymer and interacts with native integrin on the surface of MSC cells. When comparing binding to sEL polymers consisting of the RGD4CsEL, control polymer bound as well, albeit at what appears to be lower levels. This could be due to a difference in surface presentation of α_{V}β3 versus α₅β₁ or may indicate a difference in binding affinity. In addition, GRGDSPsEL (the most commonly used ‘RGD’ peptide sequence) was tested. No binding to MSC cells or to any of the recombinant integrins tested was observed (FIG. 9). This suggests that the context of the peptide matters considerably, and that selecting for binding motifs within the context of the scaffold is important. In this respect, whether the binding motif for the mscsELp46 can be isolated and confer binding out of the context of the full sEL polymer was tested. A peptide consisting of sequence AGVVVERKKCGSG, which consists of the selected binding sequence and 2-3 flanking residues but does not have the sEL sequence, was used. It was observed that an N-terminally biotinylated version of the peptide did not bind robustly to MSC cells and a non-biotinylated version did not successfully outcompete mscsELp46 for binding even at 10× concentration (FIG. 10). Altogether, this demonstrates that context is critical for adhesion, and grafting adhesion motifs selected or occurring within a different context does not always result in the desired function.

### MSC Cell Fate is Influenced by Selected Polymers

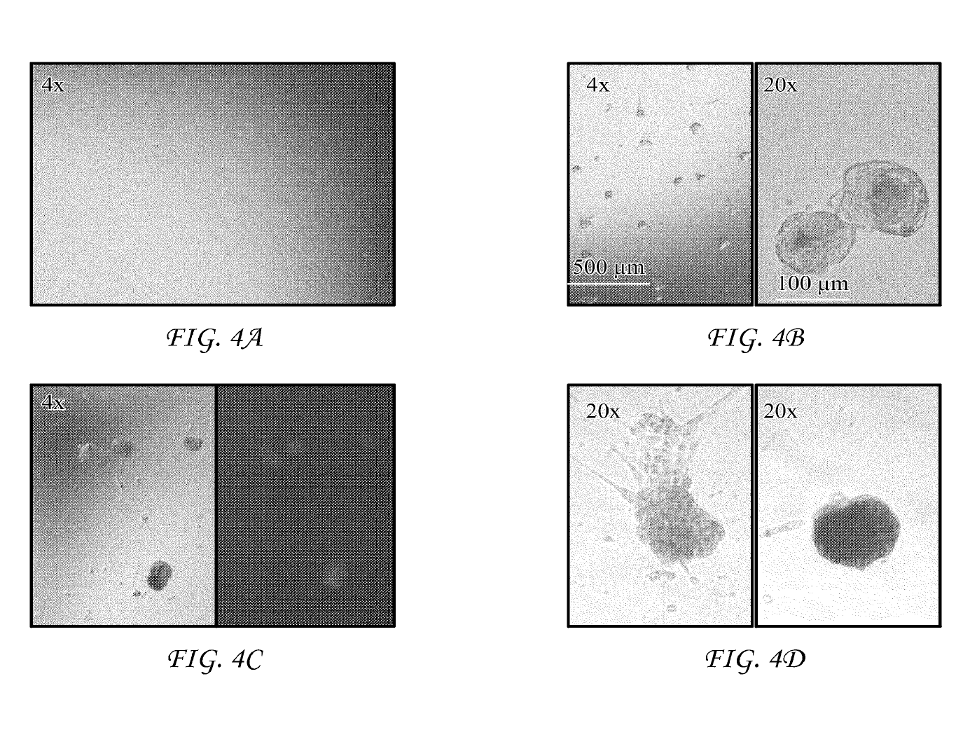

Tests were performed to evaluate if the selected polymers confer a phenotype to cells, preferably with a push towards differentiation. To test this, the selected polymers were drop cast on borosilicate chamber slides followed by seeding with AD-MSCs. Cells were seeded at relatively low density (8000-10,000 cells/cm²) and phenotypic response monitored. While a detailed phenotypic response was not performed for all of the selected polymers, after 24 hrs cells grown on mscsELp46 demonstrated obvious phenotypic characteristics relative to cells seeded on the no polymer control (NPC) or sEL-coated control plates (FIG. 4). NPC and sEL controls demonstrated typical MSC cell morphology, mscsELp46 showed distinct aggregates, or spheroids, that are morphologically similar to early chondrocytes (FIGS. 4, 5, 6), suggesting that mscsELp46 coating results in morphological or alteration in differentiation for MSC cells without the need to add chemicals and growth factors typically required for differentiation.

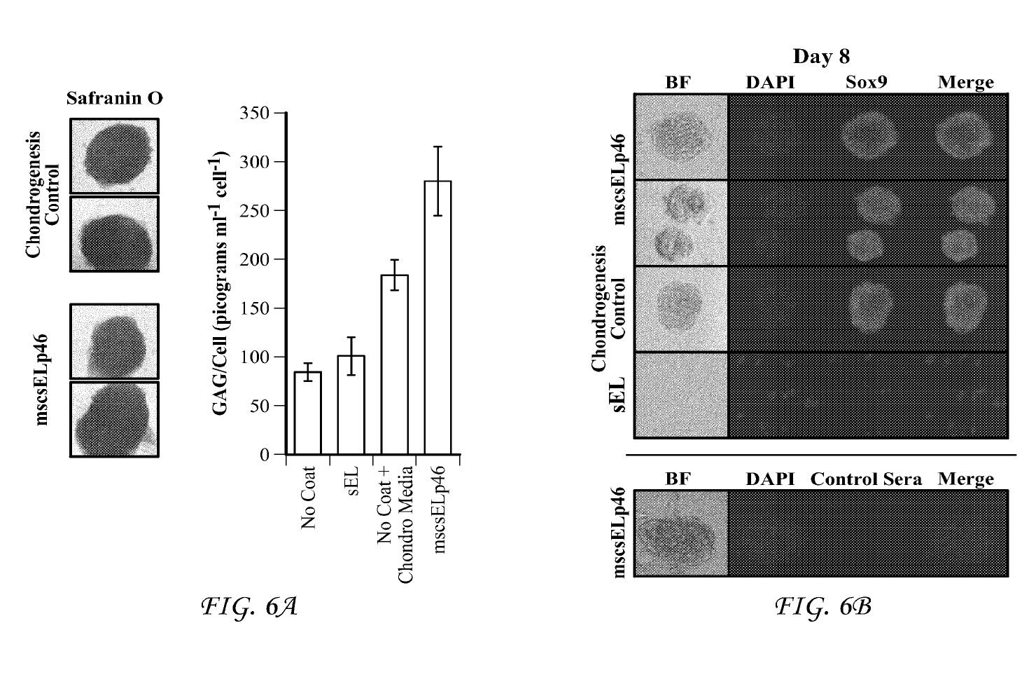

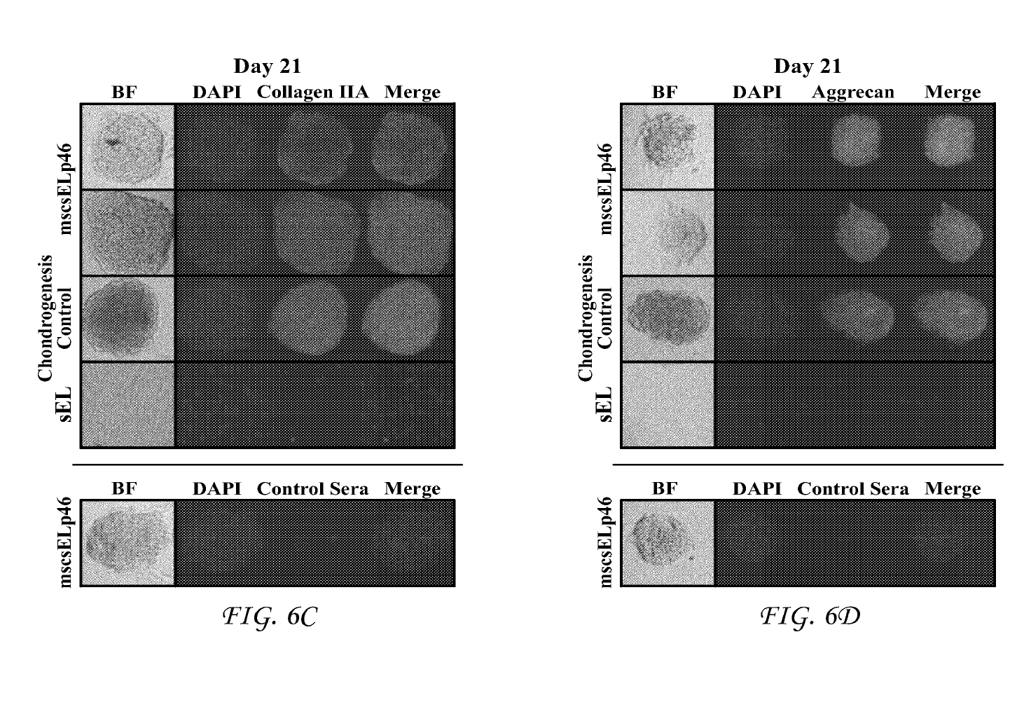

The results were even more striking when MSCs were grown in chondrogenesis medium (same seeding density on uncoated surfaces) for a few days. Spheroids grown on mscsELp46-coated plates in the presence of low fetal calf serum (2% FCS) reached a maximum diameter of <200 uM, whereas when chondrogenesis medium or a higher concentration of fetal calf serum was supplied, spheroids grew bigger (>200 uM), tended to coalesce, and often detached from the surface. When cells were seeded on dropcast mscsELp46 and chondrogenesis media was added the following day, spheroids formed immediately, more than a week before similar sized and numbers are observed for cells induced with chondrogenesis media alone or when grown on mscsELp46 without chondrogenesis media (FIGS. 5, 6). The synergistic nature of the observed response provides evidence that mscsELp46 promotes a phenotype consistent with that of cells differentiating into chondrocytes.

Tests were performed to evaluate if the spheroids observed produced proteins and surface markers indicative of chondrocyte differentiation. Production of sulfated glycosaminoglycans (GAG, dermatan, chondroitin, heparan, and keratan sulfate) and upregulation of typical chondrogenesis markers such as Collagen IIA, Aggrecan, and Sox9 were evaluated. GAG-based assays were designed to identify and quantify the production of GAG molecules, which are a hallmark of ECM production. Detection and quantification of GAG was performed by staining with Safranin O and by reaction with methylene blue, followed by measurement of absorbance at 525 nm (FIG. 6A). Aggregates stained with both Safranin O and α-Aggrecan antibody (FIG. 6A), two hallmarks of chondrogenesis. The results showed that mscsELp46-induced spheroids stained with Safranin O at levels comparable to aggregates of similar size grown in chondrogenesis media. In the quantitative GAG assay, cells grown on mscsELp46-coated surface in MSC medium, or on uncoated surface in chondrogenesis media, produced 2.8-fold and 1.8-fold higher levels of GAG per cell respectively, than cells grown on sEL-coated surface in MSC medium. Collagen IIA, Aggrecan, and Sox9 expression was assessed by immunocytochemistry, using-target specific primary antibodies followed by treatment with fluorophore-conjugated secondary antibody. Cell aggregates grown on mscsELp46-coated surfaces stained at levels comparable to aggregates induced by chondrogenesis medium (FIG. 6A). Non-aggregated, non-differentiating cells growing as monolayers did not stain with any of the antibodies used. To ensure the observed staining of spheroids was specific, normal sera (NGS) from the same animal (goat) was used as a non-specific background staining control. For all three targets, the level of staining was significantly higher than the background NGS control. Altogether, these phenotypic assays show that cells grown on mscsELp46, in the absence of any other growth factors or differentiation media, exhibit phenotypic characteristics typical of chondrocytes.

### Polymers Comprising Optical Moieties

Optical moieties, for example oligomeric phenylene vinylene (OPPV), can be coupled to the backbone of genetically encoded polymers (e.g. ELP), can be doped within ELP hydrogels, and/or can be utilized as cross-linkers between ELP polymers to create hydrogels. Here, two examples are provided. In the first type (FIG. 12A, doping the amine-containing p-phenylene vinylene oligomer into a K-ELP hydrogel created a hydrogel that is both temperature and pH stimuli responsive (resulting in an optical change), while developing emergent optical response within the hydrogel that does not occur with the OPPV alone. In the second type (FIG. 12B), amine-containing p-phenylene vinylene oligomer was utilized as a cross-linker between polymer backbones (D-sEL), resulting in a hydrogel that shows optical changes with temperature, pH and mechanical stress (FIG. 12B). Exemplary polymer sequences are as follows: AGKGSG (VPGIG)₂₅ VPASGKPIPNPLLGLDSTHHHHHH (FIG. 12A; referred to as K-sEL or K-ELP); AGDGSG (VPGIG)₂₅ VPASGKPIPNPLLGLDSTHHHHHH (FIG. 12B, D-sEL).

In this proof-of concept (FIG. 13), two types of conjugates with metal complexes as optically active moieties have been demonstrated. In the first type (FIG. 13A), direct conjugation of sEL and preformed transition-metal complexes was performed in a single step, as exemplified here for [Ru(2,2′-bipyridine)₂(1,10-phenanthrolin-5-amine)](PF₆)₂ and [Ru(1,10-phenanthroline)(1,10-phenanthrolin-5-amine)₂](PF₆)₂. These complexes were prepared by procedures similar to those reported in the literature. The amide bond coupling between sEL and the amino-functionalized complexes was performed using the 1-ethyl-3-(3-dimethylaminopropyl)carbodiimide (EDC) agent in the presence of N-hydroxysulfosuccinimide (sulfo-NHS). In a typical reaction, a solution of D-sEL (2 mg/mL) in 50 mM sodium phosphate buffer (pH 7.0) was mixed with 50-fold excess of the EDC/sulfo-NHS system for 30 min, followed by addition of the metal complex and continuous stirring for 24 h at 4° C. The conjugated products were purified by dialysis, lyophilization, and repeated washing by organic solvent. In the second type (FIG. 13B), the introduction of lanthanide complexes occurred stepwise: 1) conjugation of D-sEL and a multidentate polypyridyl ligand, as exemplified here for (2,2′:6′,2″-terpyridin)-4′-amine, and 2) subsequent complexation of the sEL-ligand conjugates with various trivalent lanthanide ions (e.g. Eu³⁺, Gd³⁺, Tb³⁺, Dy³⁺) in the form of nitrates or dibenzoylmethanates. In step 1, amide coupling was performed as above for Ru complexes.

Exemplary polymer sequences are as follows:

(D-sEL)
AGDGSG(VPGIG)₂₅VPASGKPIPNPLLGLDSTHHHHHH

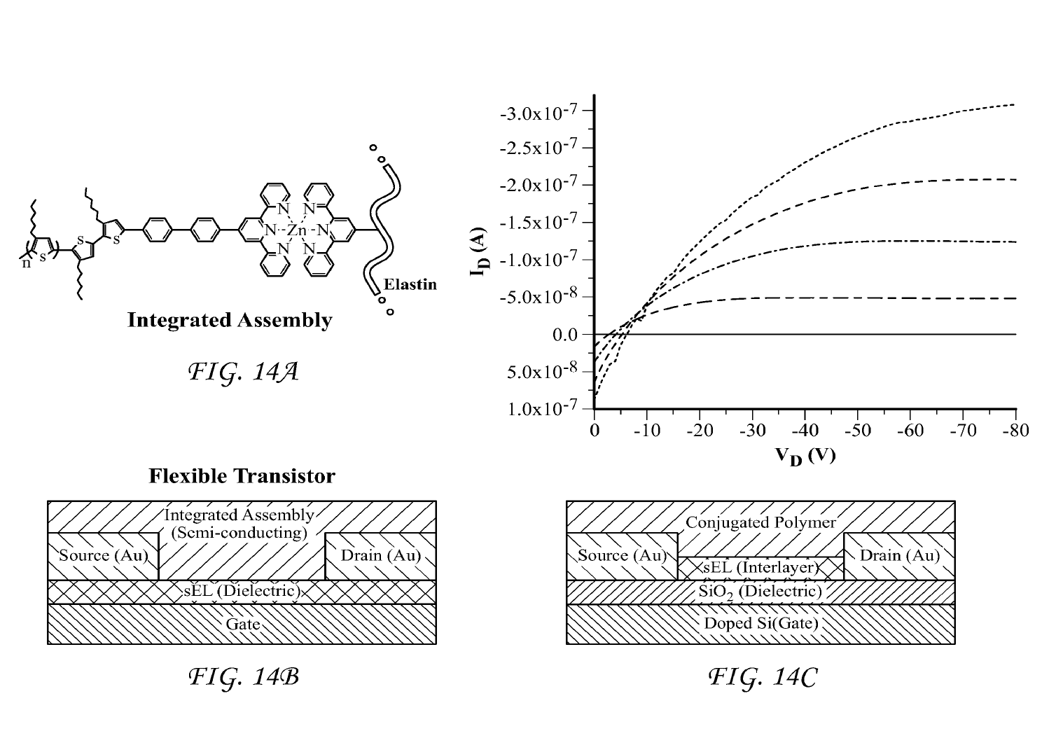

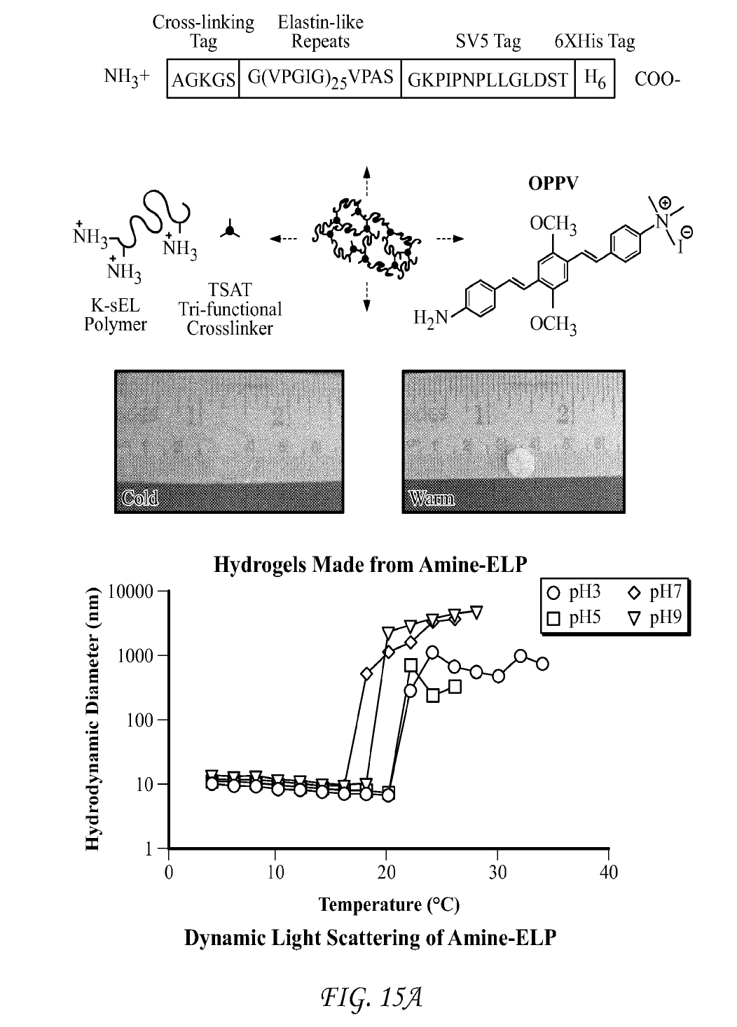

As an extension of the concept of sEL assemblies with different classes of optical materials, sEL-ligand conjugates (such as above, with (2,2′:6′,2″-terpyridin)-4′-amine) can be linked by metal coordination to another type of ligand-functionalized, exemplary, polymer (W or D-sEL) (FIG. 14). For example, terpyridine-terminated poly(3-hexylthiophene) was synthesized using externally initiated Kumada catalyst transfer polycondensation, where the polymerization was initiated from cis-chloro(o-tolyl)(dppp)nickel(II) and terminated with 4′-chloromagnesio-2,2′:6′,2″-terpyridine. By direct metal coordination of both polymers as “extended ligands”, metal-linked assemblies of the type sEL-ligand(M)ligand-polymers are achieved for a variety of applications. In FIG. 14B, an exemplary flexible transistor utilizing a semiconducting sEL (as in FIG. 10A) as the semi-conducting portion and sEL polymer as the dielectric. sEL can be utilized as a dielectric, as shown by threshold voltage (FIG. 14C). Exemplary polymer sequences are as follows: AG(VPGIG)₂₅VPASW (W-sEL or D-sEL).

Therefore, large, ELP-based libraries (>10⁸ unique sequences) can be generated and used to isolate functional polymers, and phage and yeast display can be used to down select ELP based polymers that bind to highly diverse targets from integrin proteins to Mesenchymal stem cells (MSCs). Further, a subset of these polymers selected for adhesion, can elicit a phenotypic response when used as a material for MSC growth. MSC cells grown on one of the selected polymers demonstrate phenotypic and genetic profiles consistent with differentiation towards chondrocytes.

The disclosures of each patent, patent application, and publication cited or described in this document are hereby incorporated herein by reference, in its entirety.

Those skilled in the art will appreciate that numerous changes and modifications can be made to the preferred embodiments of the disclosed methods and polymers, and that such changes and modifications can be made without departing from the spirit of the invention. It is, therefore, intended that the appended claims cover all such equivalent variations as fall within the true spirit and scope of the disclosed methods and polymers.

### EMBODIMENTS

The following list of embodiments is intended to complement, rather than displace or supersede, the previous descriptions.

### Embodiment 1

A method of identifying a polymer comprising: subjecting a plurality of first display systems to a selective pressure, wherein the first display systems contain at least one polymer exposed on the surface thereof; isolating at least one first display system that contains at least one polymer that responds to a selective pressure; isolating the DNA encoding the at least one polymer that responds to the selective pressure; and identifying the polymer that responds to the selective pressure.

### Embodiment 2

The method of Embodiment 1, wherein the selective pressure comprises binding to a target, an application of and/or a change in temperature, an application of and/or a change in force, as application of and/or a change in pressure, an application of and/or a change in pH, catalysis, an application of and/or a change in light, an application of and/or a change in current, an application of and/or a change in voltage, or any combination thereof.

### Embodiment 3

The method of Embodiment 2, wherein the selective pressure is binding to a target.

### Embodiment 4

The method of Embodiment 3, wherein the subjecting a plurality of first display systems to a select pressure step comprises incubating a target with a plurality of first display systems.

### Embodiment 5

The method of any one of the previous Embodiments, wherein the first display system is a host cell, bacteriophage, virion, or ribosome.

### Embodiment 6

The method of Embodiment 5, wherein the first display system is a host cell.

### Embodiment 7

The method of Embodiment 6, wherein the host cell is a bacteria, yeast, or virus.

### Embodiment 8

The method of any one of the previous Embodiments, wherein the identification step comprises isolating the DNA encoding the at least one polymer that responds to the selective pressure and sequencing the DNA.

### Embodiment 9

The method of any one of the previous Embodiments, further comprising: introducing the DNA into a plurality of second display systems to produce a plurality of second display systems expressing the at least one polymer on the surface thereof; subjecting the second display systems to a selective pressure; isolating at least one second display system that contains at least one polymer that responds to a selective pressure; and isolating the DNA encoding the at least one polymer that responds to the selective pressure; wherein the introducing, subjecting, and isolating steps occur prior to the identifying step.

### Embodiment 10

The method of any one of the previous Embodiments, wherein the polymer has a functional moiety.

### Embodiment 11

The method of Embodiment 10, wherein the functional moiety is an optical moiety, a transition-metal complex, a protein binding domain, or any combination thereof.

### Embodiment 12

A method of identifying a polymer comprising: incubating a target with a plurality of first host cells, wherein each host cell expresses at least one polymer; isolating at least one host cell expressing at least one polymer that binds to the target; isolating the DNA encoding the at least one polymer that binds to the target; transforming a plurality of second host cells with said DNA to produce a plurality of second host cells expressing the at least one polymer encoded by said DNA; incubating the target with the second host cells expressing the at least one polymer encoded by said DNA; isolating the second host cells expressing the at least one polymer that bind to the target; and identifying the at least one polymer that binds to the target, wherein the identification comprises isolating and sequencing the DNA from the at least one polymer expressed on the second host cells that binds to the target.

### Embodiment 13

The method of Embodiment 12, wherein the target comprises a cell, a protein, a peptide, a nucleic acid molecule, a carbohydrate, a plastic, a chemical, a drug, a pharmaceutical, or a therapeutic.

### Embodiment 14

The method of Embodiment 12 or 13, wherein the first host cells are phage.

### Embodiment 15

The method of any one of Embodiments 12-14, wherein the second host cells are yeast.

### Embodiment 16

The method of any one of Embodiments 12-15, wherein the polymers comprise at least one VPGXG unit, wherein X is any amino acid except proline.

### Embodiment 17

The method of any one of Embodiments 12-16, further comprising testing the polymers as monoclones, comprising: transforming a host cell with the identified polymer and screening said cell for a property of interest.

### Embodiment 18

The method of Embodiment 17, wherein the property of interest is binding to a cell of interest, inducing differentiation of a cell of interest, or both.

### Embodiment 19

The method of Embodiment 18, wherein the cells are stem-cells, chondrocytes, or osteoblasts.

### Embodiment 20

A method of identifying a polymer from a polymer library comprising: providing a polymer library comprising a plurality first display systems, wherein each first display system expresses at least one polymer; applying a selective pressure to the polymer library; screening the polymer library to identify one or more polymers that respond to the selective pressure; and identifying the sequence of the polymer that confers the response.

### Embodiment 21

The method of claim 20, wherein the selective pressure comprises binding to a target, an application of and/or a change in temperature, an application of and/or a change in force, as application of and/or a change in pressure, an application of and/or a change in pH, catalysis, an application of and/or a change in light, an application of and/or a change in current, an application of and/or a change in voltage, or any combination thereof.

### Embodiment 22

The method of Embodiment 20 or 21, wherein response is binding to cells, inducing the differentiation of cells, insoluble phase transition, intrinsic fluorescence, or any combination thereof.

### Embodiment 23

The method of any one of Embodiments 20-22, wherein the screening comprises evaluating binding, change in solubility, change in light emission, or any combination thereof.

### Embodiment 24

The method of any one of Embodiments 20-23, wherein the first display system is a host cell.

### Embodiment 25

The method of Embodiment 24, wherein the selective pressure is binding to a target.

### Embodiment 26

The method of any one of Embodiments 20-25, wherein identifying the sequence comprises: isolating the DNA from the host cells that express at least one polymer that responds to the selective pressure; and sequencing the DNA to identify the at least one polymer that responds to the selective pressure.

### Embodiment 27

The method of any one of Embodiments 20-26, wherein the at least one polymer has a functional moiety.

### Embodiment 28

The method of Embodiment 27, wherein the functional moiety is an optical moiety, a transition-metal complex, a protein binding domain, or any combination thereof.

### Embodiment 29

A polymer having the formula (X)(VPGIG)₂₅, wherein X is HCRGDGWLCTDK; SARYVWYNCVPIRIWR; HYYGRHWWLFHVLNYP; GYYMFSRL; GYWHYGQL; APRFRFGTMYDA; VVVERKKC; GYYMFSRL; GYWHYGQL; WHFGSLTP; APRFRFGTMYDA; WNLEPQMD; MFYEMLREWSP; RYSFGALEPISE; WKLWPMGAVPS; WYFGKME; WVLFPLGGVWS; VVVERKKC; CLLqVPWGTGTRFLTA; LCASHPLDqPVY; CHWFPRSS; FSHFVVRVNNMR; SRVDRVMV; RTWWDATTLNDY; RSAASRqKTVVV; EDPLQDGMKFqCAKVS; or LANEWqED; and wherein q represents the TAG codon that encodes Gln in E. coli.

### Embodiment 30

The polymer of Embodiment 29, further comprising an N-terminal AG, a C-terminal GSG, or both.

### Embodiment 31

A polymer library, comprising: a plurality of host cells, each host cell expressing at least one a distinct polymer.

### Embodiment 32

The polymer library of Embodiment 31, wherein said at least one polymer comprises at least one functional moiety.

### Embodiment 33

The polymer library of Embodiment 32, wherein said functional moiety is amino acid based, non-amino acid based, or a combination thereof.

### Embodiment 34

The polymer library of Embodiment 33, wherein said functional moiety is an optical moiety, a transition-metal complex, a protein binding domain, or any combination thereof.

### Embodiment 35

The polymer library of any one of Embodiments 31-34, wherein the plurality of host cells express one or more polymers of claim 25.

### Embodiment 36

The polymer library of any one of Embodiments 31-35, wherein the host cells are virus cells, yeast cells, or bacteria cells.

### Embodiment 37

A method of generating the polymer library of Embodiment 31 comprising: generating a plurality of host cells wherein each host cell expresses at least one distinct polymer.

1. A method of identifying a polymer comprising:
subjecting a plurality of first display systems to a selective pressure, wherein the first display systems contain at least one polymer exposed on the surface thereof;
isolating at least one first display system that contains at least one polymer that responds to a selective pressure;
isolating the DNA encoding the at least one polymer that responds to the selective pressure; and
identifying the polymer that responds to the selective pressure.

2. The method of claim 1, wherein the selective pressure comprises binding to a target, an application of and/or a change in temperature, an application of and/or a change in force, as application of and/or a change in pressure, an application of and/or a change in pH, catalysis, an application of and/or a change in light, an application of and/or a change in current, an application of and/or a change in voltage, or any combination thereof.

3. The method of claim 2, wherein the selective pressure is binding to a target.

4. The method of claim 3, wherein the subjecting a plurality of first display systems to a select pressure step comprises incubating a target with a plurality of first display systems.

5. The method of claim 1, wherein the first display system is a host cell, bacteriophage, virion, or ribosome.

6. The method of claim 5, wherein the first display system is a host cell.

7. The method of claim 6, wherein the host cell is a bacteria, yeast, or virus.

8. The method of claim 1, wherein the identification step comprises isolating the DNA encoding the at least one polymer that responds to the selective pressure and sequencing the DNA.

9. The method of claim 1, further comprising:
introducing the DNA into a plurality of second display systems to produce a plurality of second display systems expressing the at least one polymer on the surface thereof;
subjecting the second display systems to a selective pressure;
isolating at least one second display system that contains at least one polymer that responds to a selective pressure; and
isolating the DNA encoding the at least one polymer that responds to the selective pressure;
wherein the introducing, subjecting, and isolating steps occur prior to the identifying step.

10. The method of claim 1, wherein the polymer has a functional moiety.

11. The method of claim 10, wherein the functional moiety is an optical moiety, a transition-metal complex, a protein binding domain, or any combination thereof.

12. A method of identifying a polymer comprising:
incubating a target with a plurality of first host cells, wherein each host cell expresses at least one polymer;
isolating at least one host cell expressing at least one polymer that binds to the target;
isolating the DNA encoding the at least one polymer that binds to the target;
transforming a plurality of second host cells with said DNA to produce a plurality of second host cells expressing the at least one polymer encoded by said DNA;
incubating the target with the second host cells expressing the at least one polymer encoded by said DNA;
isolating the second host cells expressing the at least one polymer that bind to the target; and
identifying the at least one polymer that binds to the target, wherein the identification comprises isolating and sequencing the DNA from the at least one polymer expressed on the second host cells that binds to the target.

13. The method of claim 12, wherein the target comprises a cell, a protein, a peptide, a nucleic acid molecule, a carbohydrate, a plastic, a chemical, a drug, a pharmaceutical, or a therapeutic.

14. The method of claim 12, wherein the first host cells are phage.

15. The method of claim 12, wherein the second host cells are yeast.

16. The method of claim 12, wherein the polymers comprise at least one VPGXG unit, wherein X is any amino acid except proline.

17. The method of claim 12, further comprising testing the polymers as monoclones, comprising:
transforming a host cell with the identified polymer and screening said cell for a property of interest.

18. The method of claim 17, wherein the property of interest is binding to a cell of interest, inducing differentiation of a cell of interest, or both.

19. The method of claim 18, wherein the cells are stem-cells, chondrocytes, or osteoblasts.

20. A method of identifying a polymer from a polymer library comprising:
providing a polymer library comprising a plurality first display systems, wherein each first display system expresses at least one polymer;
applying a selective pressure to the polymer library;
screening the polymer library to identify one or more polymers that respond to the selective pressure; and
identifying the sequence of the polymer that confers the response.

21. The method of claim 20, wherein the selective pressure comprises binding to a target, an application of and/or a change in temperature, an application of and/or a change in force, as application of and/or a change in pressure, an application of and/or a change in pH, catalysis, an application of and/or a change in light, an application of and/or a change in current, an application of and/or a change in voltage, or any combination thereof.

22. The method of claim 20, wherein response is binding to cells, inducing the differentiation of cells, insoluble phase transition, intrinsic fluorescence, or any combination thereof.

23. The method of claim 20, wherein the screening comprises evaluating binding, change in solubility, change in light emission, or any combination thereof.

24. The method of claim 20, wherein the first display system is a host cell.

25. The method of claim 24, wherein the selective pressure is binding to a target.

26. The method of claim 20, wherein identifying the sequence comprises:
isolating the DNA from the host cells that express at least one polymer that responds to the selective pressure; and
sequencing the DNA to identify the at least one polymer that responds to the selective pressure.

27. The method of claim 20, wherein the at least one polymer has a functional moiety.

28. The method of claim 27, wherein the functional moiety is an optical moiety, a transition-metal complex, a protein binding domain, or any combination thereof.

29. A polymer having the formula
(X)(VPGIG)₂₅
wherein X is
HCRGDGWLCTDK;
SARYVWYNCVPIRIWR;
HYYGRH WWLFH VLNYP;
GYYMFSRL;
GYWHYGQL;
APRFRFGTMYDA;
VVVERKKC;
GYYMFSRL;
GYWHYGQL;
WHFGSLTP;
APRFRFGTMYDA;
WNLEPQMD;
MFYEMLREWSP;
RYSFGALEPISE;
WKLWPMGAVPS;
WYFGKME;
WVLFPLGGVWS;
VVVERKKC;
CLLqVPWGTGTRFLTA;
LCASHPLDqPVY;
CHWFPRSS;
FSHFVVRVNNMR;
SRVDRVMV;
RTWWDATTLNDY;
RSAASRqKTVVV;
EDPLQDGMKFqCAKVS;
or
LANEWqED;
and
wherein q represents the TAG codon that encodes Gin in E. coli.

30. The polymer of claim 29, further comprising an N-terminal AG, a C-terminal GSG, or both.

31. A polymer library, comprising:
a plurality of host cells, each host cell expressing at least one a distinct polymer.

32. The polymer library of claim 31, wherein said at least one polymer comprises at least one functional moiety.

33. The polymer library of claim 32, wherein said functional moiety is amino acid based, non-amino acid based, or a combination thereof.

34. The polymer library of claim 33, wherein said functional moiety is an optical moiety, a transition-metal complex, a protein binding domain, or any combination thereof.

35. The polymer library of claim 31, wherein the plurality of host cells express one or more polymers of having the formula:
(X)(VPGIG)₂₅
wherein X is
HCRGDGWLCTDK;
SARYVWYNCVPIRIWR;
HYYGRH WWLFH VLNYP;
GYYMFSRL;
GYWHYGQL;
APRFRFGTMYDA;
VVVERKKC;
GYYMFSRL;
GYWHYGQL;
WHFGSLTP;
APRFRFGTMYDA;
WNLEPQMD;
MFYEMLREWSP;
RYSFGALEPISE;
WKLWPMGAVPS;
WYFGKME;
WVLFPLGGVWS;
VVVERKKC;
CLLqVPWGTGTRFLTA;
LCASHPLDqPVY;
CHWFPRSS;
FSHFVVRVNNMR;
SRVDRVMV;
RTWWDATTLNDY;
RSAASRqKTVVV;
EDPLQDGMKFqCAKVS;
or
LANEWqED:
and
wherein q represents the TAG codon that encodes Gin in E. coli.

36. The polymer library of claim 31, wherein the host cells are virus cells, yeast cells, or bacteria cells.

37. A method of generating the polymer library of claim 31 comprising:
generating a plurality of host cells wherein each host cell expresses at least one distinct polymer.


SEQUENCE LISTING (117 sequences)

SEQ ID NO: 1 - protein, 141 aa, Artificial Sequence
  Description of Artificial Sequence Synthetic polypeptide
XXXXXXXXXXXXXXXXVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 2 - protein, 12 aa, Artificial Sequence
  Description of Artificial Sequence Synthetic peptide
HCRGDGWLCTDK

SEQ ID NO: 3 - protein, 16 aa, Artificial Sequence
  Description of Artificial Sequence Synthetic peptide
SARYVWYNCVPIRIWR

SEQ ID NO: 4 - protein, 16 aa, Artificial Sequence
  Description of Artificial Sequence Synthetic peptide
HYYGRHWWLFHVLNYP

SEQ ID NO: 5 - protein, 8 aa, Artificial Sequence
  Description of Artificial Sequence Synthetic peptide
GYYMFSRL

SEQ ID NO: 6 - protein, 8 aa, Artificial Sequence
  Description of Artificial Sequence Synthetic peptide
GYWHYGQL

SEQ ID NO: 7 - protein, 12 aa, Artificial Sequence
  Description of Artificial Sequence Synthetic peptide
APRFRFGTMYDA

SEQ ID NO: 8 - protein, 8 aa, Artificial Sequence
  Description of Artificial Sequence Synthetic peptide
VVVERKKC

SEQ ID NO: 9 - protein, 8 aa, Artificial Sequence
  Description of Artificial Sequence Synthetic peptide
GYYMFSRL

SEQ ID NO: 10 - protein, 8 aa, Artificial Sequence
  Description of Artificial Sequence Synthetic peptide
GYWHYGQL

SEQ ID NO: 11 - protein, 8 aa, Artificial Sequence
  Description of Artificial Sequence Synthetic peptide
WHFGSLTP

SEQ ID NO: 12 - protein, 12 aa, Artificial Sequence
  Description of Artificial Sequence Synthetic peptide
APRFRFGTMYDA

SEQ ID NO: 13 - protein, 8 aa, Artificial Sequence
  Description of Artificial Sequence Synthetic peptide
WNLEPQMD

SEQ ID NO: 14 - protein, 11 aa, Artificial Sequence
  Description of Artificial Sequence Synthetic peptide
MFYEMLREWSP

SEQ ID NO: 15 - protein, 12 aa, Artificial Sequence
  Description of Artificial Sequence Synthetic peptide
RYSFGALEPISE

SEQ ID NO: 16 - protein, 11 aa, Artificial Sequence
  Description of Artificial Sequence Synthetic peptide
WKLWPMGAVPS

SEQ ID NO: 17 - protein, 7 aa, Artificial Sequence
  Description of Artificial Sequence Synthetic peptide
WYFGKME

SEQ ID NO: 18 - protein, 11 aa, Artificial Sequence
  Description of Artificial Sequence Synthetic peptide
WVLFPLGGVWS

SEQ ID NO: 19 - protein, 8 aa, Artificial Sequence
  Description of Artificial Sequence Synthetic peptide
VVVERKKC

SEQ ID NO: 20 - protein, 16 aa, Artificial Sequence
  Description of Artificial Sequence Synthetic peptide
CLLQVPWGTGTRFLTA

SEQ ID NO: 21 - protein, 12 aa, Artificial Sequence
  Description of Artificial Sequence Synthetic peptide
LCASHPLDQPVY

SEQ ID NO: 22 - protein, 8 aa, Artificial Sequence
  Description of Artificial Sequence Synthetic peptide
CHWFPRSS

SEQ ID NO: 23 - protein, 12 aa, Artificial Sequence
  Description of Artificial Sequence Synthetic peptide
FSHFVVRVNNMR

SEQ ID NO: 24 - protein, 8 aa, Artificial Sequence
  Description of Artificial Sequence Synthetic peptide
SRVDRVMV

SEQ ID NO: 25 - protein, 12 aa, Artificial Sequence
  Description of Artificial Sequence Synthetic peptide
RTWWDATTLNDY

SEQ ID NO: 26 - protein, 12 aa, Artificial Sequence
  Description of Artificial Sequence Synthetic peptide
RSAASRQKTVVV

SEQ ID NO: 27 - protein, 16 aa, Artificial Sequence
  Description of Artificial Sequence Synthetic peptide
EDPLQDGMKFQCAKVS

SEQ ID NO: 28 - protein, 8 aa, Artificial Sequence
  Description of Artificial Sequence Synthetic peptide
LANEWQED

SEQ ID NO: 29 - protein, 6 aa, Artificial Sequence
  Description of Artificial Sequence Synthetic peptide
GRGDSP

SEQ ID NO: 30 - protein, 6 aa, Artificial Sequence
  Description of Artificial Sequence Synthetic peptide
LCPTSR

SEQ ID NO: 31 - protein, 5 aa, Artificial Sequence
  Description of Artificial Sequence Synthetic peptide
VPGXG

SEQ ID NO: 32 - protein, 10 aa, Artificial Sequence
  Description of Artificial Sequence Synthetic peptide
VPGXGVPGXG

SEQ ID NO: 33 - protein, 25 aa, Artificial Sequence
  Description of Artificial Sequence Synthetic peptide
VPGXGVPGXGVPGXGVPGXGVPGXG

SEQ ID NO: 34 - protein, 50 aa, Artificial Sequence
  Description of Artificial Sequence Synthetic polypeptide
VPGXGVPGXGVPGXGVPGXGVPGXGVPGXGVPGXGVPGXGVPGXGVPGXG

SEQ ID NO: 35 - protein, 75 aa, Artificial Sequence
  Description of Artificial Sequence Synthetic polypeptide
VPGXGVPGXGVPGXGVPGXGVPGXGVPGXGVPGXGVPGXGVPGXGVPGXGVPGXGVPGXGVPGXGVPGXGVPGXG

SEQ ID NO: 36 - protein, 100 aa, Artificial Sequence
  Description of Artificial Sequence Synthetic polypeptide
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 37 - protein, 125 aa, Artificial Sequence
  Description of Artificial Sequence Synthetic polypeptide
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 38 - protein, 125 aa, Artificial Sequence
  Description of Artificial Sequence Synthetic polypeptide
VPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 39 - protein, 6 aa, Artificial Sequence
  Description of Artificial Sequence Synthetic peptide
GAGAGS

SEQ ID NO: 40 - protein, 5 aa, Artificial Sequence
  Description of Artificial Sequence Synthetic peptide
GPGGX

SEQ ID NO: 41 - protein, 15 aa, Artificial Sequence
  Description of Artificial Sequence Synthetic peptide
GGRPSDSYGAPGGGN

SEQ ID NO: 42 - protein, 13 aa, Artificial Sequence
  Description of Artificial Sequence Synthetic peptide
GYSGGRPGGQDLG

SEQ ID NO: 43 - protein, 136 aa, Artificial Sequence
  Description of Artificial Sequence Synthetic polypeptide
AGLCPTSRGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 44 - protein, 6 aa, Artificial Sequence
  Description of Artificial Sequence Synthetic peptide
VPGIGX

SEQ ID NO: 45 - protein, 7 aa, Artificial Sequence
  Description of Artificial Sequence Synthetic peptide
GAGAGSX

SEQ ID NO: 46 - protein, 11 aa, Artificial Sequence
  Description of Artificial Sequence Synthetic peptide
VPGIGVPGVGX

SEQ ID NO: 47 - protein, 11 aa, Artificial Sequence
  Description of Artificial Sequence Synthetic peptide
GAGASVPGIGX

SEQ ID NO: 48 - protein, 146 aa, Artificial Sequence
  Description of Artificial Sequence Synthetic polypeptide
XXXXXXXXXXXXXXXXXXXXXVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 49 - protein, 17 aa, Artificial Sequence
  Description of Artificial Sequence Synthetic peptide
AGHCRGDGWLCTDKGSG

SEQ ID NO: 50 - protein, 21 aa, Artificial Sequence
  Description of Artificial Sequence Synthetic peptide
AGSARYVWYNCVPIRIWRGSG

SEQ ID NO: 51 - protein, 21 aa, Artificial Sequence
  Description of Artificial Sequence Synthetic peptide
AGHYYGRHWWLFHVLNYPGSG

SEQ ID NO: 52 - protein, 13 aa, Artificial Sequence
  Description of Artificial Sequence Synthetic peptide
AGGYYMFSRLGSG

SEQ ID NO: 53 - protein, 13 aa, Artificial Sequence
  Description of Artificial Sequence Synthetic peptide
AGGYWHYGQLGSG

SEQ ID NO: 54 - protein, 17 aa, Artificial Sequence
  Description of Artificial Sequence Synthetic peptide
AGAPRFRFGTMYDAGSG

SEQ ID NO: 55 - protein, 13 aa, Artificial Sequence
  Description of Artificial Sequence Synthetic peptide
AGVVVERKKCGSG

SEQ ID NO: 56 - protein, 13 aa, Artificial Sequence
  Description of Artificial Sequence Synthetic peptide
AGGYYMFSRLGSG

SEQ ID NO: 57 - protein, 13 aa, Artificial Sequence
  Description of Artificial Sequence Synthetic peptide
AGGYWHYGQLGSG

SEQ ID NO: 58 - protein, 13 aa, Artificial Sequence
  Description of Artificial Sequence Synthetic peptide
AGWHFGSLTPGSG

SEQ ID NO: 59 - protein, 17 aa, Artificial Sequence
  Description of Artificial Sequence Synthetic peptide
AGAPRFRFGTMYDAGSG

SEQ ID NO: 60 - protein, 13 aa, Artificial Sequence
  Description of Artificial Sequence Synthetic peptide
AGWNLEPQMDGSG

SEQ ID NO: 61 - protein, 16 aa, Artificial Sequence
  Description of Artificial Sequence Synthetic peptide
AGMFYEMLREWSPGSG

SEQ ID NO: 62 - protein, 17 aa, Artificial Sequence
  Description of Artificial Sequence Synthetic peptide
AGRYSFGALEPISEGSG

SEQ ID NO: 63 - protein, 16 aa, Artificial Sequence
  Description of Artificial Sequence Synthetic peptide
AGWKLWPMGAVPSGSG

SEQ ID NO: 64 - protein, 12 aa, Artificial Sequence
  Description of Artificial Sequence Synthetic peptide
AGWYFGKMEGSG

SEQ ID NO: 65 - protein, 16 aa, Artificial Sequence
  Description of Artificial Sequence Synthetic peptide
AGWVLFPLGGVWSGSG

SEQ ID NO: 66 - protein, 13 aa, Artificial Sequence
  Description of Artificial Sequence Synthetic peptide
AGVVVERKKCGSG

SEQ ID NO: 67 - protein, 21 aa, Artificial Sequence
  Description of Artificial Sequence Synthetic peptide
AGCLLQVPWGTGTRFLTAGSG

SEQ ID NO: 68 - protein, 17 aa, Artificial Sequence
  Description of Artificial Sequence Synthetic peptide
AGLCASHPLDQPVYGSG

SEQ ID NO: 69 - protein, 13 aa, Artificial Sequence
  Description of Artificial Sequence Synthetic peptide
AGCHWFPRSSGSG

SEQ ID NO: 70 - protein, 17 aa, Artificial Sequence
  Description of Artificial Sequence Synthetic peptide
AGFSHFVVRVNNMRGSG

SEQ ID NO: 71 - protein, 13 aa, Artificial Sequence
  Description of Artificial Sequence Synthetic peptide
AGSRVDRVMVGSG

SEQ ID NO: 72 - protein, 17 aa, Artificial Sequence
  Description of Artificial Sequence Synthetic peptide
AGRTWWDATTLNDYGSG

SEQ ID NO: 73 - protein, 17 aa, Artificial Sequence
  Description of Artificial Sequence Synthetic peptide
AGRSAASRQKTVVVGSG

SEQ ID NO: 74 - protein, 21 aa, Artificial Sequence
  Description of Artificial Sequence Synthetic peptide
AGEDPLQDGMKFQCAKVSGSG

SEQ ID NO: 75 - protein, 13 aa, Artificial Sequence
  Description of Artificial Sequence Synthetic peptide
AGLANEWQEDGSG

SEQ ID NO: 76 - protein, 27 aa, Artificial Sequence
  Description of Artificial Sequence Synthetic peptide
KLAKFYFKETVIMKKIWLALAGLVLAF

SEQ ID NO: 77 - protein, 9 aa, Artificial Sequence
  Description of Artificial Sequence Synthetic peptide
SAHAAGGSG

SEQ ID NO: 78 - protein, 129 aa, Artificial Sequence
  Description of Artificial Sequence Synthetic polypeptide
VPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPAS

SEQ ID NO: 79 - protein, 14 aa, Artificial Sequence
  Description of Artificial Sequence Synthetic peptide
GKPIPNPLLGLDST

SEQ ID NO: 80 - protein, 6 aa, Artificial Sequence
  Description of Artificial Sequence Synthetic 6xHis tag
HHHHHH

SEQ ID NO: 81 - protein, 17 aa, Artificial Sequence
  Description of Artificial Sequence Synthetic peptide
MKYLLPTAAAGLLLLAA

SEQ ID NO: 82 - protein, 10 aa, Artificial Sequence
  Description of Artificial Sequence Synthetic peptide
SGAHAAGGSG

SEQ ID NO: 83 - protein, 85 aa, Artificial Sequence
  Description of Artificial Sequence Synthetic polypeptide
MRFPSIFTAVLFAASSALAAPANTTTEDETAQIPAEAVIDYSDLEGDFDAAALPLSNSTNNGLSSTNTTIASIAAKEEGVQLDKR

SEQ ID NO: 84 - protein, 9 aa, Artificial Sequence
  Description of Artificial Sequence Synthetic peptide
GAHAAGGSG

SEQ ID NO: 85 - protein, 15 aa, Artificial Sequence
  Description of Artificial Sequence Synthetic peptide
GGGGSGGGGSGGGGS

SEQ ID NO: 86 - protein, 69 aa, Artificial Sequence
  Description of Artificial Sequence Synthetic polypeptide
QELTTICEQIPSPTLESTPYSLSTTTILANGKAMQGVFEYYKSVTFVSNCGSHPSTTSKGSPINTQYVF

SEQ ID NO: 87 - protein, 9 aa, Artificial Sequence
  Description of Artificial Sequence Synthetic peptide
KDNSSTIEG

SEQ ID NO: 88 - protein, 12 aa, Artificial Sequence
  Description of Artificial Sequence Synthetic peptide
RPYDVPDYALQA

SEQ ID NO: 89 - protein, 18 aa, Artificial Sequence
  Description of Artificial Sequence Synthetic peptide
SGGGGSGGGGSGGGGSAR

SEQ ID NO: 90 - DNA, 78 bp, Artificial Sequence
  Description of Artificial Sequence Synthetic oligonucleotide
cgtttagcgcgcatgccgccggannknnknnknnknnknnknnknnkggctctggtgtaccaggtatcggtgtccccg

SEQ ID NO: 91 - DNA, 90 bp, Artificial Sequence
  Description of Artificial Sequence Synthetic oligonucleotide
cgtttagcgcgcatgccgccggannknnknnknnknnknnknnknnknnknnknnknnkggctctggtgtaccaggtatcggtgtccccg

SEQ ID NO: 92 - DNA, 102 bp, Artificial Sequence
  Description of Artificial Sequence Synthetic polynucleotide
cgtttagcgcgcatgccgccggannknnknnknnknnknnknnknnknnknnknnknnknnknnknnknnkggctctggtgtaccaggtatcggtgtccccg

SEQ ID NO: 93 - DNA, 26 bp, Artificial Sequence
  Description of Artificial Sequence Synthetic oligonucleotide
cagtgggtttgggattggtttgccgc

SEQ ID NO: 94 - DNA, 26 bp, Artificial Sequence
  Description of Artificial Sequence Synthetic oligonucleotide
gcggcaaaccaatcccaaacccactg

SEQ ID NO: 95 - DNA, 23 bp, Artificial Sequence
  Description of Artificial Sequence Synthetic oligonucleotide
tccggcggcatgcgcgctaaacg

SEQ ID NO: 96 - DNA, 41 bp, Artificial Sequence
  Description of Artificial Sequence Synthetic oligonucleotide
cctctctatgggcagtcggtgattctggctggcgctggcag

SEQ ID NO: 97 - DNA, 45 bp, Artificial Sequence
  Description of Artificial Sequence Synthetic oligonucleotide
cctctctatgggcagtcggtgattgccaaagaagaaggagtccag

SEQ ID NO: 98 - DNA, 62 bp, Artificial Sequence
  Description of Artificial Sequence Synthetic oligonucleotide
ttccatctcatccctgcgtgtctccgactcagnnnnnnnnnngtaccccgatcccaggaact

SEQ ID NO: 99 - DNA, 62 bp, Artificial Sequence
  Description of Artificial Sequence Synthetic oligonucleotide
gccaaagaagaaggagtccagttagataaaagaggcgcgctggcgtttagcgcgcatgccgc

SEQ ID NO: 100 - DNA, 74 bp, Artificial Sequence
  Description of Artificial Sequence Synthetic oligonucleotide
ctcctgttgaatctaatcctaataatgggtttgggataggctttccgctagcaggtaccccaatccccggcaca

SEQ ID NO: 101 - protein, 6 aa, Artificial Sequence
  Description of Artificial Sequence Synthetic peptide
RGDGWL

SEQ ID NO: 102 - protein, 142 aa, Artificial Sequence
  Description of Artificial Sequence Synthetic polypeptide
AGHCRGDGWLCTDKGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 103 - protein, 146 aa, Artificial Sequence
  Description of Artificial Sequence Synthetic polypeptide
AGSARYVWYNCVPIRIWRGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 104 - protein, 146 aa, Artificial Sequence
  Description of Artificial Sequence Synthetic polypeptide
AGHYYGRHWWLFHVLNYPGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 105 - protein, 138 aa, Artificial Sequence
  Description of Artificial Sequence Synthetic polypeptide
AGGYYMFSRLGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 106 - protein, 138 aa, Artificial Sequence
  Description of Artificial Sequence Synthetic polypeptide
AGGYWHYGQLGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 107 - protein, 142 aa, Artificial Sequence
  Description of Artificial Sequence Synthetic polypeptide
AGAPRFRFGTMYDAGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 108 - protein, 138 aa, Artificial Sequence
  Description of Artificial Sequence Synthetic polypeptide
AGVVVERKKCGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 109 - protein, 24 aa, Artificial Sequence
  Description of Artificial Sequence Synthetic peptide
VPASGKPIPNPLLGLDSTHHHHHH

SEQ ID NO: 110 - protein, 5 aa, Artificial Sequence
  Description of Artificial Sequence Synthetic peptide
AGKGS

SEQ ID NO: 111 - protein, 155 aa, Artificial Sequence
  Description of Artificial Sequence Synthetic polypeptide
AGKGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPASGKPIPNPLLGLDSTHHHHHH

SEQ ID NO: 112 - protein, 155 aa, Artificial Sequence
  Description of Artificial Sequence Synthetic polypeptide
AGDGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPASGKPIPNPLLGLDSTHHHHHH

SEQ ID NO: 113 - protein, 132 aa, Artificial Sequence
  Description of Artificial Sequence Synthetic polypeptide
AGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPASW

SEQ ID NO: 114 - protein, 21 aa, Artificial Sequence
  Description of Artificial Sequence Synthetic peptide
AGXXXXXXXXXXXXXXXXGSG

SEQ ID NO: 115 - protein, 25 aa, Artificial Sequence
  Description of Artificial Sequence Synthetic peptide
GAHAAGXXXXXXXXXXXXXXXXGSG

SEQ ID NO: 116 - protein, 134 aa, Artificial Sequence
  Description of Artificial Sequence Synthetic polypeptide
LCPTSRGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG

SEQ ID NO: 117 - protein, 134 aa, Artificial Sequence
  Description of Artificial Sequence Synthetic polypeptide
LGPTSRGSGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIG